SEQUENCE LISTING (151 sequences)

SEQ ID NO: 1 - protein, 16 aa, Triticum aestivum
ELQPFPQPELPYPQPQ

SEQ ID NO: 2 - protein, 15 aa, Hordeum vulgare
EQPFPQPEQPFPWQP

SEQ ID NO: 3 - protein, 16 aa, Hordeum vulgare
EPEQPIPEQPQPYPQQ

SEQ ID NO: 4 - protein, 17 aa, Triticum aestivum
QLQPFPQPQLPYPQPQS

SEQ ID NO: 5 - protein, 15 aa, Triticum aestivum
QQCCQPLAQISEQAR

SEQ ID NO: 6 - protein, 11 aa, Hordeum vulgare
MVLQTLPSMCR

SEQ ID NO: 7 - protein, 8 aa, Hordeum vulgare
PQQPFPQQ

SEQ ID NO: 8 - protein, 16 aa, Hordeum vulgare
FVQPQQQPFPLQPHQP

SEQ ID NO: 9 - protein, 15 aa, Hordeum vulgare
YPEQPQQPFPWQQPT

SEQ ID NO: 10 - protein, 19 aa, Hordeum vulgare
LERPQQLFPQWQPLPQQPP

SEQ ID NO: 11 - protein, 18 aa, Hordeum vulgare
LIIPQQPQQPFPLQPHQP

SEQ ID NO: 12 - protein, 5 aa, Secale cereale
QQPFP

SEQ ID NO: 13 - protein, 5 aa, Secale cereale
QQQFP

SEQ ID NO: 14 - protein, 5 aa, Secale cereale
LQPFP

SEQ ID NO: 15 - protein, 5 aa, Secale cereale
QLPFP

SEQ ID NO: 16 - protein, 16 aa, Hordeum vulgare
RVVDQQLVGQLPWSTG

SEQ ID NO: 17 - protein, 16 aa, Hordeum vulgare
VVDQQLVGQLPWSTGL

SEQ ID NO: 18 - protein, 15 aa, Hordeum vulgare
VVDQQLVGQLPWSTG

SEQ ID NO: 19 - protein, 12 aa, Hordeum vulgare
VVDQQLVGQLPW

SEQ ID NO: 20 - protein, 12 aa, Hordeum vulgare
QLVGQLPWSTGL

SEQ ID NO: 21 - protein, 11 aa, Hordeum vulgare
QLVGQLPWSTG

SEQ ID NO: 22 - protein, 13 aa, Hordeum vulgare
LVGQLPWSTGLQM

SEQ ID NO: 23 - protein, 11 aa, Hordeum vulgare
LVGQLPWSTGL

SEQ ID NO: 24 - protein, 10 aa, Hordeum vulgare
LVGQLPWSTG

SEQ ID NO: 25 - protein, 12 aa, Hordeum vulgare
VGQLPWSTGLQM

SEQ ID NO: 26 - protein, 10 aa, Hordeum vulgare
VGQLPWSTGL

SEQ ID NO: 27 - protein, 15 aa, Hordeum vulgare
  MOD_RES (1)..(1): Pyroglutamic acid
  MOD_RES (15)..(15): HexNAc
QLAAQLPAMCRLEGS

SEQ ID NO: 28 - protein, 26 aa, Hordeum vulgare
VVRQYEQQTEVPSKGGSFYPGGTAPP

SEQ ID NO: 29 - protein, 15 aa, Hordeum vulgare
FVLPQQQAQFKVVGS

SEQ ID NO: 30 - protein, 10 aa, Hordeum vulgare
FVLPQQQAQF

SEQ ID NO: 31 - protein, 16 aa, Hordeum vulgare
AIVMQQQVQQQVGHGF

SEQ ID NO: 32 - protein, 19 aa, Hordeum vulgare
LERPQQLFPQWQPLPQQPP

SEQ ID NO: 33 - protein, 13 aa, Hordeum vulgare
LFPQWQPLPQQPP

SEQ ID NO: 34 - protein, 12 aa, Hordeum vulgare
LQQLGQGMPIQL

SEQ ID NO: 35 - protein, 9 aa, Hordeum vulgare
VVGSLVIQT

SEQ ID NO: 36 - protein, 19 aa, Hordeum vulgare
LQQPQHQFPQPTQQFPQRP

SEQ ID NO: 37 - protein, 15 aa, Hordeum vulgare
YPEQPQQPFPWQQPT

SEQ ID NO: 38 - protein, 12 aa, Hordeum vulgare
GVVQPQQLAQME

SEQ ID NO: 39 - protein, 17 aa, Hordeum vulgare
  MOD_RES (1)..(1): Pyroglutamic acid
QPQHQFPQPTQQFPQRP

SEQ ID NO: 40 - protein, 14 aa, Hordeum vulgare
FVQPQQQVPVEITR

SEQ ID NO: 41 - protein, 12 aa, Hordeum vulgare
FVQPQQQVPVEI

SEQ ID NO: 42 - protein, 18 aa, Hordeum vulgare
LIIPQQPQQPFPLQPHQP

SEQ ID NO: 43 - protein, 17 aa, Hordeum vulgare
IIPQQPQQPFPLQPHQP

SEQ ID NO: 44 - protein, 12 aa, Hordeum vulgare
VQVQIPFVHPSI

SEQ ID NO: 45 - protein, 15 aa, Hordeum vulgare
SFGQPQQQVPVEVMR

SEQ ID NO: 46 - protein, 28 aa, Hordeum vulgare
IIPQQPFPLQPQPFPQQPQQPLPQPQQP

SEQ ID NO: 47 - protein, 14 aa, Hordeum vulgare
  MOD_RES (6)..(6): dansyl-glutamic acid (DEA)
IPLQPQQPFPQQPP

SEQ ID NO: 48 - protein, 14 aa, Hordeum vulgare
TLPTMCSVNVPLYR

SEQ ID NO: 49 - protein, 16 aa, Hordeum vulgare
DVSPECRPVALSQVVR

SEQ ID NO: 50 - protein, 15 aa, Hordeum vulgare
QQCCQQLANINEQSR

SEQ ID NO: 51 - protein, 17 aa, Hordeum vulgare
CTAIDSIVHAIFMQQGR

SEQ ID NO: 52 - protein, 15 aa, Hordeum vulgare
QQCCQPLAQISEQAR

SEQ ID NO: 53 - protein, 11 aa, Hordeum vulgare
VFLQQQCSPVR

SEQ ID NO: 54 - protein, 15 aa, Hordeum vulgare
VFLQQQCSPVPMPQR

SEQ ID NO: 55 - protein, 18 aa, Hordeum vulgare
QLNPSHQELQSPQQPFLK

SEQ ID NO: 56 - protein, 11 aa, Hordeum vulgare
ELQESSLEACR

SEQ ID NO: 57 - protein, 13 aa, Hordeum vulgare
APFVGVVTGVGGQ

SEQ ID NO: 58 - protein, 15 aa, Hordeum vulgare
QQCCQQLANINEQSR

SEQ ID NO: 59 - protein, 13 aa, Hordeum vulgare
APFVGVVTGVGGQ

SEQ ID NO: 60 - DNA, 23 bp, Artificial Sequence
  Oligonucleotide primer
gacacatattctgccaaaacccc

SEQ ID NO: 61 - DNA, 20 bp, Artificial Sequence
  Oligonucleotide primer
acgagggcgacgattaccgc

SEQ ID NO: 62 - DNA, 25 bp, Artificial Sequence
  Oligonucleotide primer
gagatcaattcattgacagtccacc

SEQ ID NO: 63 - DNA, 24 bp, Artificial Sequence
  Oligonucleotide primer
cttgtcctgactgctgcggagaaa

SEQ ID NO: 64 - DNA, 25 bp, Artificial Sequence
  Oligonucleotide primer
gcaacaaggacactacccaagtatg

SEQ ID NO: 65 - DNA, 25 bp, Artificial Sequence
  Oligonucleotide primer
gctgacaatgagctgagacatgtag

SEQ ID NO: 66 - DNA, 19 bp, Artificial Sequence
  Oligonucleotide primer
ggcaatacgagcagcaaac

SEQ ID NO: 67 - DNA, 20 bp, Artificial Sequence
  Oligonucleotide primer
cctctgtcctggttgttgtc

SEQ ID NO: 68 - DNA, 20 bp, Artificial Sequence
  Oligonucleotide primer
tcgcaggatcctgtacaacg

SEQ ID NO: 69 - DNA, 20 bp, Artificial Sequence
  Oligonucleotide primer
caacaatgaagaccttcctc

SEQ ID NO: 70 - DNA, 22 bp, Artificial Sequence
  Oligonucleotide primer
cgagaaggtaccattactccag

SEQ ID NO: 71 - DNA, 21 bp, Artificial Sequence
  Oligonucleotide primer
agtaacaatgaaggtccatcg

SEQ ID NO: 72 - DNA, 2838 bp, Hordeum vulgare
gacacatattctgccaaaaccccagaacaataatcacttctcgtagatgaagagaacagaccaagatacaaacgtccacgcttcagcaaacagtaccccagaactaggattaagccgattacgcggctttagcagaccgtccaaaaaaactgttttgcaaagctccaattcctccttgcttatccaatttcttttgtgttggcaaactgcacttgtccaaccgattttgttcttcccgtgtttcttcttaggctaactaacacagccgtgcacatagccatggtccggaatcttcacctcgtccctataaaagcccagccaatctccacaatctcatcatcaccgagaacaccgagaaccacaaaactagagatcaattcattgacagtccaccgagatggctaagcggctggtcctctttgtggcggtaatcgtcgccctcgtggctctcaccaccgctgaacgtgagatcaatgggaacaacattttccttgatagccgctctaggcagctacagtgtgagcgcgagctccaggagagctcgctcgaggcgtgccggcgggtcgtggaccaacagctggttggccagctgccatggagcacggggctccagatgcagtgctgccagcagcttcgggacgtcagccccgagtgccgccccgtcgccctcagccaggtcgtgaggcaatacgagcagcaaaccgaggtgccatccaagggaggatccttctacccgggcgggaccgcaccgccgctgcagcaaggaggatggtggggaacctctgtaaaatggtactacccagaccaaacttcttcgcaacagtcatggcaagggcaacaagggtaccaccaaagcgtaacttcttcccagcagccaggacaagggcagcaagggtcctacccaggttcaactttcccgcagcagccaggacaaggacaacaaccaggacagaggcagccatggtcctatccaagtgcaactttcccacaacagccagggcaagggcaagggcaacaagggtactacccaggcgcaacttccctgctgcagccaggacaagggcaacaagggccctaccagagtgcaacttctccacagcagccaggacaaggacagggacaccaagagacctatcaatttgcaacttccccgcatcagccaggacaatggcaacaaccaggacaagggcaacaagggtactacccaagtgtaacttctccacaacagtcgggacaagggcaaacagggtacccaagtacaacttctccacaacaatcggggcaagggcaacagctgggacaagggcaacaaccaggacaagggcaacaagggtacccaagtgcaacttttccacaacagccaggacaatggcaacaagggtcctacccaagtacaacttctccgcagcagtcaggacaagggcaacaagggtacaacccaagtggaacttctacgcagcagccgggacaagtgcaacagttgggacaagggcaacaagggtactacccaattgcaacttctccgcagcagccaggacaagggcaacagctaggacaagggcaacaaccaggacatgggcaacagctagtgcaagggcaacaacaaggacaagggcaacaaggacactacccaagtatgacttctccgcaccaaacaggacaagggcaaaaaggatactacccaagtgcaatttctccgcagcagtcaggacaaggacaacaaggataccagcctagtggagcttcttcacaggggtcggtgcaaggggcgtgccagcacagcacatcttctccgcagcagcaagcacaagggtgccaagcttcttcaccaaagcaagggctagggtcgttgtactacccgagtggagcttatacacaacagaaaccagggcaagggtacaacccaggtggaacttctccgctgcaccagcaagggggagggttcggcggcgggttaacgacggagcaaccgcagggaggaaagcagccattccattgccagcaaaccactgtctcccctcaccagggtcagcaaaccactgtttcccctcatcagggtcagcaaaccactgtctcccctcatcagggtcagcaaaccactgtctcccctcaccagggtcagcaaaccaccgtctcccctcaccagggtcagcaaaccaccgtctcccctcatcagggtcagcaaaccactgtctcccctcatccgggtcagcaaaccactgtctcccctcatcagggtcagcaaaccactgtctcccctcatccgggtcagcaaaccactgtctcccctcatcagggtcagcaaaccactgtctcccctcatcagggtcagcaaaccaccgtctcccctcatcagggtcagcaaaccaccgtctcccctcatcagggtcagcaaaccaccgtctcccctcatcagggtcagcagcccggcgagcagccttgcggtttccctggccagcaaaccaccgtgtctctgcaccatggtcagcagtccaacgagttgtactacggcagcccataccatgttagcgtggagcagccgtcggccagcctaaaggtagcaaaggcgcagcagctcgcggcgcagctgccggcaatgtgtcggctggagggcggcggcggcctgttggccagccagtagtagaactctggcagctcgcatggtgcttgggcatgcatgcatcttagctatacaataaacgtgacgtgtgcttgcagtttttcatgtaactagggtaaaacccaacaataatgcaaaacggaaagcttctccatccaaaaaaagaacaaaactggtgctatatatagtatgcgctacatgtctcagctcattgtcag

SEQ ID NO: 73 - DNA, 2724 bp, Hordeum vulgare
gacacatattctgccaaaaccccagaacaataatcacttctcgtagatgaagagaacagaccaagatacaaacgtccacgcttcagcaaacagtaccccagaactaggattaagccgattacgcggctttagcagaccgtccaaaaaaactgttttgcaaagctccaattcctccttgcttatccaatttcttttgtgttggcaaactgcacttctccaaccgattttgttcttcccatgtttcttcttaggctaactaacacagccgtgcacatagccatggtccggaaccttcacctcgtccctataaaagcccagccaatctccacaatctcatcatcaccgagaacaccgagaaccacaaaactagagatcaattcattgacagtccaccgagatggctaagcggctggtcctctttgtggcggtaatcgtcgccctcgtggctctcaccaccgctgaacctgagatcaatgggaacaacattttccttgatagccgctctgggcagctacagtgtgagcgcgagctccaggagagctcgctcgaggcgtgccggcgggtcgtggaccaacagctggttggccagctgccatggagcacggggctccagatgcagtgctgccagcagcttcgggacgtcagccccgagtgccgccccgtcgccctcagccaggtcgtgaggcaatacgagcagcaaaccgaggtgccatccaagggaggatccttctacccgggcgggaccgcaccgccgctgcagcaaggaggatggtggggaacctctgtaaaatggtactacccagaccaaacttcttcgcaacagtcatggcaagggcaacaagggtagcaccaaagcgtaacttcttcccagcagccaggacaagggcagcaagggtcctacccaggttcaactttcccgcagcagccaggacaaggacaacaaccaggacagaggcagccatggtcctatccaagtgcaactttcccacaacagccagggcaagggcaagggcaagggcaacaagggtactacccaggcgcaacttccctgctgcagccaggacaagggcaacaagggccctaccaaagtgcaacttctccacagcagccaggacaaggacagggacaacaagagccctatccaattgcaacttccccgcatcagccaggacaatggcaacaaccaggacaagggcaacaagggtactacccaagtgtaacttctccacaacagtcgggacaagggcaacaagggtacccaagtacaacttctccacaacaatcggggcaagggcaacagctgggacaagggcaacaaccaggacaagggcaacaagggtacccaagtgcaacttttccacaacagccaggacaatggcaacaagggtcctacccaagtacaacttctccgcagcagtcaggacaagggcaacaagggtacaacccaagtggaacttctacgcagcagccgggacaagtgcaacagttgggacaagggcaacaagggtactacccaattgcaacttctccgcagcagccaggacaagggcaacagctaggacaagggcaacaaccaggacatgggcaacagctagtgcaagggcaacaacaaggacaagggcaacaaggacactacccaagtatgacttctccgcaccaaacaggacaagggcaaaaaggatactacccaagtgcaatttctccgcagcagtcaggacaaggacaacaaggataccagcctagtggagcttcttcacaggggtcggtgcaaggggcgtaccagcacagcacatcttctccgcagcagcaagcacaagggtgccaagcttcttcaccaaagcaagggctagggtcgttgtactacccgagtggagcttatacacaacagaaaccagggcaagggtacaacccaggtggaacttctccgctgcaccagcaagggggagggttcggcggcgggttaacgacggagcaaccgcagggaggaaagcagccattccattgccagcaaaccactgtctcccctcaccagggtcagcaaaccactgtctcccctcatcagggtcagcaaaccactgtctcccctcatcagggtcagcaaaccactgtctcccctcaccagggtcagcaaaccactgtctcccctcatccgggtcagcaaaccactgtctcccctcatcagggtcagcaaaccactgtctcccctcatccgggtcagcaaaccaccgtctcccctcatcagggtcagcaaaccaccgtctcccctcatcagggtcagcaaaccaccgtctcccctcaccagggtcagcagcccggcgagcagccttgcggtttccctggccagcaaaccaccgtgtctctgcaccatggtcagcagtccaacgagttgtactacggcagcccataccatgttagcgtggagcagccgtcggccagcctaaaggtagcaaaggcgcagcagctcgcggcacagctgccggcaatgtgtcggctggagggcggcggcggcctgttggccagccagtagtagaactctggcagctcgcatggtgcttgggcatgcatgcaccttagctatacaataaacgtgacgtgtgcttgcagtttttcatgtaactagggtaaaacccaacaataatgcaaaacggaaagcttctccatccaaaaaaagaacaaaactggtgctatatatagtatgcgctacatgtctcagctcattgtcag

SEQ ID NO: 74 - protein, 747 aa, Hordeum vulgare
MAKRLVLFVAVIVALVALTTAEREINGNNIFLDSRSRQLQCERELQESSLEACRRVVDQQLVGQLPWSTGLQMQCCQQLRDVSPECRPVALSQVVRQYEQQTEVPSKGGSFYPGGTAPPLQQGGWWGTSVKWYYPDQTSSQQSWQGQQGYHQSVTSSQQPGQGQQGSYPGSTFPQQPGQGQQPGQRQPWSYPSATFPQQPGQGQGQQGYYPGATSLLQPGQGQQGPYQSATSPQQPGQGQGHQETYQFATSPHQPGQWQQPGQGQQGYYPSVTSPQQSGQGQTGYPSTTSPQQSGQGQQLGQGQQPGQGQQGYPSATFPQQPGQWQQGSYPSTTSPQQSGQGQQGYNPSGTSTQQPGQVQQLGQGQQGYYPIATSPQQPGQGQQLGQGQQPGHGQQLVQGQQQGQGQQGHYPSMTSPHQTGQGQKGYYPSAISPQQSGQGQQGYQPSGASSQGSVQGACQHSTSSPQQQAQGCQASSPKQGLGSLYYPSGAYTQQKPGQGYNPGGTSPLHQQGGGFGGGLTTEQPQGGKQPFHCQQTTVSPHQGQQTTVSPHQGQQTTVSPHQGQQTTVSPHQGQQTTVSPHQGQQTTVSPHQGQQTTVSPHPGQQTTVSPHQGQQTTVSPHPGQQTTVSPHQGQQTTVSPHQGQQTTVSPHQGQQTTVSPHQGQQTTVSPHQGQQPGEQPCGFPGQQTTVSLHHGQQSNELYYGSPYHVSVEQPSASLKVAKAQQLAAQLPAMCRLEGGGGLLASQ

SEQ ID NO: 75 - protein, 149 aa, Hordeum vulgare
MAKRLVLFVAVIVALVALTTAEPEINGNNIFLDSRSGQLQCERELQESSLEACRRVVDQQLVGQLPWSTGLQMQCCQQLRDVSPECRPVALSQVVRQYEQQTEVPSKGGSFYPGGTAPPLQQGGWWGTSVKWYYPDQTSSQQSWQGQQG

SEQ ID NO: 76 - DNA, 2244 bp, Hordeum vulgare
atggctaagcggctggtcctctttgtggcggtaatcgtcgccctcgtggctctcaccaccgctgaacgtgagatcaatgggaacaacattttccttgatagccgctctaggcagctacagtgtgagcgcgagctccaggagagctcgctcgaggcgtgccggcgggtcgtggaccaacagctggttggccagctgccatggagcacggggctccagatgcagtgctgccagcagcttcgggacgtcagccccgagtgccgccccgtcgccctcagccaggtcgtgaggcaatacgagcagcaaaccgaggtgccatccaagggaggatccttctacccgggcgggaccgcaccgccgctgcagcaaggaggatggtggggaacctctgtaaaatggtactacccagaccaaacttcttcgcaacagtcatggcaagggcaacaagggtaccaccaaagcgtaacttcttcccagcagccaggacaagggcagcaagggtcctacccaggttcaactttcccgcagcagccaggacaaggacaacaaccaggacagaggcagccatggtcctatccaagtgcaactttcccacaacagccagggcaagggcaagggcaacaagggtactacccaggcgcaacttccctgctgcagccaggacaagggcaacaagggccctaccagagtgcaacttctccacagcagccaggacaaggacagggacaccaagagacctatcaatttgcaacttccccgcatcagccaggacaatggcaacaaccaggacaagggcaacaagggtactacccaagtgtaacttctccacaacagtcgggacaagggcaaacagggtacccaagtacaacttctccacaacaatcggggcaagggcaacagctgggacaagggcaacaaccaggacaagggcaacaagggtacccaagtgcaacttttccacaacagccaggacaatggcaacaagggtcctacccaagtacaacttctccgcagcagtcaggacaagggcaacaagggtacaacccaagtggaacttctacgcagcagccgggacaagtgcaacagttgggacaagggcaacaagggtactacccaattgcaacttctccgcagcagccaggacaagggcaacagctaggacaagggcaacaaccaggacatgggcaacagctagtgcaagggcaacaacaaggacaagggcaacaaggacactacccaagtatgacttctccgcaccaaacaggacaagggcaaaaaggatactacccaagtgcaatttctccgcagcagtcaggacaaggacaacaaggataccagcctagtggagcttcttcacaggggtcggtgcaaggggcgtgccagcacagcacatcttctccgcagcagcaagcacaagggtgccaagcttcttcaccaaagcaagggctagggtcgttgtactacccgagtggagcttatacacaacagaaaccagggcaagggtacaacccaggtggaacttctccgctgcaccagcaagggggagggttcggcggcgggttaacgacggagcaaccgcagggaggaaagcagccattccattgccagcaaaccactgtctcccctcaccagggtcagcaaaccactgtttcccctcatcagggtcagcaaaccactgtctcccctcatcagggtcagcaaaccactgtctcccctcaccagggtcagcaaaccaccgtctcccctcaccagggtcagcaaaccaccgtctcccctcatcagggtcagcaaaccactgtctcccctcatccgggtcagcaaaccactgtctcccctcatcagggtcagcaaaccactgtctcccctcatccgggtcagcaaaccactgtctcccctcatcagggtcagcaaaccactgtctcccctcatcagggtcagcaaaccaccgtctcccctcatcagggtcagcaaaccaccgtctcccctcatcagggtcagcaaaccaccgtctcccctcatcagggtcagcagcccggcgagcagccttgcggtttccctggccagcaaaccaccgtgtctctgcaccatggtcagcagtccaacgagttgtactacggcagcccataccatgttagcgtggagcagccgtcggccagcctaaaggtagcaaaggcgcagcagctcgcggcgcagctgccggcaatgtgtcggctggagggcggcggcggcctgttggccagccagtag

SEQ ID NO: 77 - DNA, 450 bp, Hordeum vulgare
atggctaagcggctggtcctctttgtggcggtaatcgtcgccctcgtggctctcaccaccgctgaacctgagatcaatgggaacaacattttccttgatagccgctctgggcagctacagtgtgagcgcgagctccaggagagctcgctcgaggcgtgccggcgggtcgtggaccaacagctggttggccagctgccatggagcacggggctccagatgcagtgctgccagcagcttcgggacgtcagccccgagtgccgccccgtcgccctcagccaggtcgtgaggcaatacgagcagcaaaccgaggtgccatccaagggaggatccttctacccgggcgggaccgcaccgccgctgcagcaaggaggatggtggggaacctctgtaaaatggtactacccagaccaaacttcttcgcaacagtcatggcaagggcaacaagggtag

SEQ ID NO: 78 - protein, 181 aa, Hordeum vulgare
LACEVLKERSVCILQLHYVQPSILQQLNPCKVFLQQQCSPVRMPQLIARSQMLQQSSCHVLQQQCCQQLPQIPEQFRHEAIRAIVYSIFLQEQPQQSVQGASQPQQQLQEEQVGQCYFQQPQPQQLGQPQQVPQSVFLQPHQIAQLEATNSIALRTLPTMCNVNVPLYDIMPFGVGTRVGV

SEQ ID NO: 79 - protein, 274 aa, Hordeum vulgare
MKTFLIFALLAIAATNTIAQQQPFPQQPQPYPQQPQPYPQQPFPPQQPFPQQPPFWWQQPVQSQQQPCQQQQTPLPQGQQYQPLLQQQIPFVHPSVLQQLNPCKVFLQQQCSPVPMPQRIARSQMLQQSSCHVLQQQCCKQLPQIPEQFRHEAIRAIIYSIILQEQQQVQDFVQPQQQQPQQSVQGVSQSQQQSQQPQLGQCSFQQPQLQQLGQQPQQQQVPLWAFLQPQQMAQLEVMTSVALRTLPTMCNVNVPLYGITTSVPLSVGTGVGPY

SEQ ID NO: 80 - protein, 347 aa, Hordeum vulgare
  misc_feature (161)..(161): Xaa can be any naturally occurring amino acid
MKTFLTFVLLAMAMSIVTTARQLNPSHQELQSPQQPFLKQQSYLQQPYPQQPYLPQQPFPTPQQFFPYLPQQTFPPSQQPNPLQPQQPFPLQPQPPQQPFPQPQQPNPQQPQQPFPRQPQQIVPQQPQQPFPQQPQQPFPQPQQPFSWQPQQPFLQPLQLXPLQAQQPFPLQPQLPFPQPQQPIGQQPKQPLLQQPQQTIPQQPQQPFPLQPQQPFPQQPQQPLPQQPQQIISQQPQQPFPLQPQQPFPQPQPFPQEQPQQAFPLQPQQPFPEESEQIITQQPFPLQPQQLFPQQPQQPLPQPQQPFRQLPKYIIPQQPQQPFLLQPHQPQQPYAQQDIWSDIALLG

SEQ ID NO: 81 - protein, 305 aa, Hordeum vulgare
MKILIILTILAMATTFATSEMQVNPSVQVQPTQQQPYPESQQPFISQSQQQFPQPQQPFPQQPQQPFPQSQQQCLQQPQHQFPQPTQQFPQRPLLPFTHPFLTFPDQLLPQPPHQSFPQPPQSYPQPPLQPFPQPPQQKYPEQPQQPFPWQQPTIQLYLQQQLNPCKEFLLQQCRPVSLLSYIWSKIVQQSSCRVMQQQCCLQLAQIPEQYKCTAIDSIVHAIFMQQGQRQGVQIVQQQPQPQQVGQCVLVQGQGVVQPQQLAQMEAIRTLVLQSVPSMCNFNVPPNCSTIKAPFVGVVTGVGGQ

SEQ ID NO: 82 - protein, 255 aa, Hordeum vulgare
MQVNPSVQVQPTQQQPYPESQQPFISQSQQQFPQPQQPFPQRPLLPFTHPFLTFPDQLLPQPPHQSFPQPPQSYPQPPLQPFPQPPQQKYPEQPQQPFPWQQPTIQLYLQQQLNPYKEFLLQQCRPVSLLSYLWSKIVQQSSCRVMLQQCCLQLAQIPEQYKCTAIDSIVHAIFMQQGQRQGVQIVQQQPQPQQVGQCVLVQGQGVVQPQQLAQMEAIRTLVLQSVPSMCNFNVPPNCSTIKAPFVGVVTGVGGQ

SEQ ID NO: 83 - protein, 289 aa, Hordeum vulgare
ITTTTMQFNPSGLELERPQQLFPQWQPLPQQPPFLQQEPEQPYPQQQPLPQQQPFPQQPQLPHQHQFPQQLPQQQFPQQMPLQPQQQFPQQMPLQPQQQPQFPQQKPFGQYQQPLTQQPYPQQQPLAQQQPSIEEQHQLNLCKEFLLQQCTLDEKVPLLQSVISFLRPHISQQNSCQLKRQQCCQQLANINEQSRCPAIQTIVHAIVMQQQVQQQVGHGFVQSQLQQLGQGMPIQLQQQPGQAFVLPQQQAQFKVVGSLVIQTLPMLCNVHVPPYCSPFGSMATGSGGQ

SEQ ID NO: 84 - protein, 173 aa, Hordeum vulgare
MKTMLILALIAFAATSAVAQLDTTCSQGYGQCQQQPQQQMNTCAAFLQQCSRTPYVQSQMWQASGCQLMRQQCCQPLAQISEQARCQAVCSMAQVIMRQQQGQSFTQPQQQQSQSFGQPQQQVPVEVMRMVLQTLPSMCSVNIPQYCTTTPCSTITPTIYSIPMAATCAGGVC

SEQ ID NO: 85 - protein, 13 aa, Hordeum vulgare
AQQLAAQLPAMCR

SEQ ID NO: 86 - protein, 148 aa, Hordeum vulgare
MAFKYQLLLSAAVMLAILVATATSFGDSCAPGDALPHNPLRACRTYVVSQICHQGPRLLTSDMKRRCCDELSAIPAYCRCEALRIIMQGVVTWQGAFEGAYFKDSPNCPRERQTSYAANLVTPQECNLGTIHGSAYCPELQPGYGVVL

SEQ ID NO: 87 - protein, 224 aa, Hordeum vulgare
MGKFIFFAVFLTTLMTISAAQGVLEQSLTDAQCRGEVQAKPLLACRQILEHQLTGRAVGVRPFQAQWGARDRCCQQLESVSHGCRCSALRGMVRDYEQSMPPLREGRRRSSGERQQEQGCSGESTAEQQQEVQGGQYGSETGESQQQQGGGYHGVTVGRGGQQQGQMLCRERPQRQQQGEGFSGEGAQQKPKVGRVRLTKVRLPTACRIEPQECSVFSTLPVLG

SEQ ID NO: 88 - protein, 164 aa, Hordeum vulgare
MASKMVVYAVLLLLPMLTATSVAVDQGSMVSNSPGEWCWPGMGYPVYPFPRCRALVKSQCAGGQVVESIQKDCCRQIAAIGDEWCICGALGSMRGSMYKELGVALADDKATVAEVFPGCRTEVMDRAVASLPAVCNQYIPNTNGTDGVCYWLSYYQPPRQMSSR

SEQ ID NO: 89 - protein, 152 aa, Hordeum vulgare
MGAMWMKSMLLVLLLCMLMVTPMTGARSDNSGPWMWCDPEMGHKVSPLTRCRALVKLECVGNRVPEDVLRDCCQEVANISNEWCRCGDLGSMLRSVYAALGVGGGPEEVFPGCQKDVMKLLVAGVPALCNVPIPNEAAGTRGVCYWSASTDT

SEQ ID NO: 90 - protein, 146 aa, Hordeum vulgare
MASSQLVPWALLLAVLTTVVATAERDYGEYCRVGKSIPINPLPACREYITRRCAVGDQQVPDVLKQQCCRELSDLPESCRCDALSILVNGVITEDGSRVGRMEAVPGCDRERIHSMGSYLTAYSECNLHNPGTPGGDCVLFGGGIS

SEQ ID NO: 91 - protein, 171 aa, Hordeum vulgare
MACKSSRSLLLLATVMVSVFAAAAAAAAATDCSPGVAFPTNLLGHCRDYVLQQTCAVFTPGSKLPEWMTSAELNYPGQPYLAKLYCCQELAEIPQQCRCEALRYFMALPVPSQPVDPSTGNVGQSGLMDLPGCPREMQRDFVRLLVAPGQCNLATIHNVRYCPAVEQPLWI

SEQ ID NO: 92 - protein, 145 aa, Hordeum vulgare
MASKSSITPLLLAAVLASVFAAAAATGQYCYAGMGLPSNPLEGCREYVAQQTCGVSIAGSPVSTEPGDTPKDRCCQELDEAPQHCRCEAVRYFIGRRSHPDWSVLKDLPGCPKEPQRDFAKVLVTPGQCNVLTVHNAPYCLGLDI

SEQ ID NO: 93 - protein, 149 aa, Hordeum vulgare
MASKSSCDLLLAAVLVSIFAAVAAVGSEDCTPWTATPITPLPSCRDYVEQQACRIETPGPPYLAKQQCCGELANIPQQCRCQALRFFMGRKSRPDQSGLMELPGCPREVQMDFVRILVTPGFCNLTTVHNTPYCLAMDEWQWNRQFCSS

SEQ ID NO: 94 - protein, 143 aa, Hordeum vulgare
MASCSQHLLSAVAIFSVLAGVATATSIYTCYEGMGLPVNPLQGCRFYVASQTCGAVPLLPIEVMKDWCCRELAGISSNCRCEGLRVFIDRAFPPSQSQGAPPQLPPLATECPAEVKRDFARTLALPGQCNLPAIHGGAYCVFP

SEQ ID NO: 95 - protein, 199 aa, Hordeum vulgare
MASLPTSSVLLPILLLVLVAATADAATFTVINKCQYTVWAAAVPAGGGQKLDAGQTWSINVPAGTTSGRVWARTGCSFDGAGNGRCQTGDCGGKLRCTQYGQAPNTLAEFGLNKYMGQDFFDISLIDGYNVPMSFVPAPGSTGCPKGGPRCPKVITPACPNELRAAGGCNNACTVFKEDRYCCTGSAANSCGPTDYSRF

SEQ ID NO: 96 - protein, 233 aa, Hordeum vulgare
MEHFCFLVILSLSGLAMALQLTTPCNAAQAQPQPIYDTDGHELTGNNLYNIMPVGRNLSDQCVSPSSFREDQCRVRAILTPCKQFRGTDGYVRIRLAEVSDSSDTEALPRLSTDVVIDFRGVVTSCVYPLQWYVRQENTKQMHVTAGLLTGMDGCKTPAGTCLAEIFMFRVEKHGAGYKLMWCPDAPCRDLVLLDYAGRRYLTVEKDGREPLVVVFKKFHRVTLPPASHPQLG

SEQ ID NO: 97 - protein, 203 aa, Hordeum vulgare
MGSRRAGLLLLSLILASTALSRSADPPPVHDTDGHELRADANYYVLSANRAHGGGLTMAPGHGRHCPLFVSQDPNGQHDGFPVRITPYGVAPSDKIIRLSTDVRISFRAYTTCLQSTEWHIDSELAAGRRHVITGPVKDPSPSGRENAFRIEKYSGAEVHEYKLMSCGDWCQDLGVFRDLKGGAWFLGATEPYHVVVFKKAPP

SEQ ID NO: 98 - protein, 16 aa, Hordeum vulgare
DYVLQQTCAVFTPGSK

SEQ ID NO: 99 - protein, 17 aa, Hordeum vulgare
LLVAPGQCNLATIHNVR

SEQ ID NO: 100 - protein, 15 aa, Hordeum vulgare
LYCCQELAEIPQQCR

SEQ ID NO: 101 - protein, 9 aa, Hordeum vulgare
CGDLGSMLR

SEQ ID NO: 102 - protein, 15 aa, Hordeum vulgare
DCCQEVANISNEWCR

SEQ ID NO: 103 - protein, 7 aa, Hordeum vulgare
LECVGNR

SEQ ID NO: 104 - protein, 22 aa, Hordeum vulgare
LLVAGVPALCNVPIPNEAAGTR

SEQ ID NO: 105 - protein, 21 aa, Hordeum vulgare
SVYAALGVGGGPEEVFPGCQK

SEQ ID NO: 106 - protein, 7 aa, Hordeum vulgare
VPEDVLR

SEQ ID NO: 107 - protein, 9 aa, Hordeum vulgare
DYVEQQACR

SEQ ID NO: 108 - protein, 8 aa, Hordeum vulgare
EVQMDFVR

SEQ ID NO: 109 - protein, 11 aa, Hordeum vulgare
IETPGPPYLAK

SEQ ID NO: 110 - protein, 15 aa, Hordeum vulgare
QQCCGELANIPQQCR

SEQ ID NO: 111 - protein, 16 aa, Hordeum vulgare
SRPDQSGLMELPGCPR

SEQ ID NO: 112 - protein, 26 aa, Hordeum vulgare
AFPPSQSQGAPPQLPPLATECPAEVK

SEQ ID NO: 113 - protein, 10 aa, Hordeum vulgare
ELAGISSNCR

SEQ ID NO: 114 - protein, 20 aa, Hordeum vulgare
FYVASQTCGAVPLLPIEVMK

SEQ ID NO: 115 - protein, 13 aa, Hordeum vulgare
CCDELSAIPAYCR

SEQ ID NO: 116 - protein, 7 aa, Hordeum vulgare
DSPNCPR

SEQ ID NO: 117 - protein, 7 aa, Hordeum vulgare
LLTSDMK

SEQ ID NO: 118 - protein, 13 aa, Hordeum vulgare
TYVVSQICHQGPR

SEQ ID NO: 119 - protein, 13 aa, Hordeum vulgare
CCQELDEAPQHCR

SEQ ID NO: 120 - protein, 26 aa, Hordeum vulgare
EYVAQQTCGVSIAGSPVSTEPGDTPK

SEQ ID NO: 121 - protein, 9 aa, Hordeum vulgare
SHPDWSVLK

SEQ ID NO: 122 - protein, 13 aa, Hordeum vulgare
CAVGDQQVPDVLK

SEQ ID NO: 123 - protein, 18 aa, Hordeum vulgare
CDALSILVNGVITEDGSR

SEQ ID NO: 124 - protein, 7 aa, Hordeum vulgare
DYGEYCR

SEQ ID NO: 125 - protein, 10 aa, Hordeum vulgare
ELSDLPESCR

SEQ ID NO: 126 - protein, 9 aa, Hordeum vulgare
MEAVPGCDR

SEQ ID NO: 127 - protein, 11 aa, Hordeum vulgare
SIPINPLPACR

SEQ ID NO: 128 - protein, 14 aa, Hordeum vulgare
SQCAGGQVVESIQK

SEQ ID NO: 129 - protein, 11 aa, Hordeum vulgare
ATVAEVFPGCR

SEQ ID NO: 130 - protein, 10 aa, Hordeum vulgare
ELGVALADDK

SEQ ID NO: 131 - protein, 19 aa, Hordeum vulgare
QIAAIGDEWCICGALGSMR

SEQ ID NO: 132 - protein, 10 aa, Hordeum vulgare
DLVLLDYAGR

SEQ ID NO: 133 - protein, 8 aa, Hordeum vulgare
EPLVVVFK

SEQ ID NO: 134 - protein, 10 aa, Hordeum vulgare
LSTDVVIDFR

SEQ ID NO: 135 - protein, 13 aa, Hordeum vulgare
AAGGCNNACTVFK

SEQ ID NO: 136 - protein, 18 aa, Hordeum vulgare
CTQYGQAPNTLAEFGLNK

SEQ ID NO: 137 - protein, 17 aa, Hordeum vulgare
CQYTVWAAAVPAGGGQK

SEQ ID NO: 138 - protein, 19 aa, Hordeum vulgare
LDAGQTWSINVPAGTTSGR

SEQ ID NO: 139 - protein, 12 aa, Hordeum vulgare
TGCSFDGAGNGR

SEQ ID NO: 140 - protein, 11 aa, Hordeum vulgare
VITPACPNELR

SEQ ID NO: 141 - protein, 12 aa, Hordeum vulgare
ADANYYVLSANR

SEQ ID NO: 142 - protein, 21 aa, Hordeum vulgare
AYTTCLQSTEWHIDSELAAGR

SEQ ID NO: 143 - protein, 18 aa, Hordeum vulgare
GGAWFLGATEPYHVVVFK

SEQ ID NO: 144 - protein, 20 aa, Hordeum vulgare
HCPLFVSQDPNGQHDGFPVR

SEQ ID NO: 145 - protein, 8 aa, Hordeum vulgare
HVITGPVK

SEQ ID NO: 146 - protein, 11 aa, Hordeum vulgare
ITPYGVAPSDK

SEQ ID NO: 147 - protein, 16 aa, Hordeum vulgare
SADPPPVHDTDGHELR

SEQ ID NO: 148 - protein, 10 aa, Hordeum vulgare
YSGAEVHEYK

SEQ ID NO: 149 - protein, 10 aa, Hordeum vulgare
DYEQSMPPLR

SEQ ID NO: 150 - protein, 10 aa, Hordeum vulgare
QILEHQLTGR

SEQ ID NO: 151 - protein, 17 aa, Hordeum vulgare
QQQGEGFSGEGAQQKPK

(19) United States
(12) Patent Application Publication
(10) Pub. No.: US 2023/0089545 A1
(43) Pub. Date: Mar. 23, 2023

(54) BARLEY WITH REDUCED LEVELS OF ALPHA-AMYLASE/TRYPSIN INHIBITORS

(71) Applicant: Commonwealth Scientific and Industrial Research Organisation, Acton (AU)

(72) Inventors: Michelle Lisa COLGRAVE, Acton (AU); Alexander Crispin HOWITT, Acton (AU); Utpal BOSE, Acton (AU)

(73) Assignee: Commonwealth Scientific and Industrial Research Organisation, Acton (AU)

(21) Appl. No.: 17/797,780

(22) PCT Filed: Feb. 4, 2021
(86) PCT No.: PCT/AU2021/050086

§ 371 Date: Aug. 5, 2022

(30) Foreign Application Priority Data
Feb. 5, 2020 (AU) 2020900312

(51) Int. Cl. C12C 1/18; A01H 6/46; A21D 13/047; A01H 5/10; A21D 2/38; A23L 33/00; A23L 7/20; C07K 14/415; C12C 12/00

(52) U.S. Cl. CPC C12C 1/18; A01H 6/4624; A21D 13/047; A01H 5/10; A21D 2/38; A23L 33/00; A23L 7/20; C07K 14/415; C12C 12/00; C12C 2200/01

(57) ABSTRACT

The present invention relates to methods of producing a food or malt-based beverage suitable for consumption by a subject with a non-coeliac gastrointestinal sensitivity. In particular, the present invention relates to methods of producing a food or malt-based beverage from barley grain comprising reduced levels of one or more alpha-amylase/trypsin inhibitors. Also provided is barley grain that can be used in the methods of the invention.

### FIELD OF THE INVENTION

The present invention relates to methods of producing a food or malt-based beverage suitable for consumption by a subject with a non-coeliac gastrointestinal sensitivity. In particular, the present invention relates to methods of producing a food or malt-based beverage from barley grain comprising reduced levels of one or more alpha-amylase/trypsin inhibitors. Also provided is barley grain that can be used in the methods of the invention.

### BACKGROUND OF THE INVENTION

Grains have been consumed as part of the human regular diet for over 10,000 years with evidence of grain cultivation dating back to ancient Mesopotamia. It is estimated that ˜1% of the world population are affected by coeliac disease (CD), a multi-factorial disease triggered upon ingestion of grains such as wheat, barley and rye (Catassi et al., 2015; Ludvigsson et al., 2013; Sapone et al., 2012).

Distinct to CD, non-coeliac gastrointestinal sensitivity, such as non-coeliac gluten sensitivity (NCGS), has received attention in recent years due to its ability to initiate intestinal (diarrhea, abdominal pain and bloating) and extra-intestinal symptoms (foggy mind, headache, fatigue, joint and muscle pain, leg or arm numbness) upon ingestion of grains (Casella et al., 2016; Catassi et al., 2013).

Non-coeliac gluten sensitivity (NCGS) is estimated to affect ˜10% of the world population. Diagnosis of NCGS is a complex processes which can involve multiple tests. To confirm the presence of NCGS in patients, the serological test should be negative to CD and IgE-mediated wheat allergy (Catassi et al., 2015). At present, avoiding grains such as wheat, barley and rye is the only treatment available for NCGS patients. Several protagonists such as fermentable oligo-, di- and monosaccharides and polyols (FODMAPs), microbial lipopolysaccharide (LPS), intestinal alkaline phosphatase and wheat alpha-amylase trypsin inhibitors (ATIs) have been proposed to play a role in the pathogenesis of NCGS.

In planta ATIs may have a role in grain filling and maturation (Finnie et al., 2002), but can also inhibit parasite amylase and trypsin-like activities (Barber et al., 1989; Bellinghausen et al., 2018; Finnie et al., 2002) and as such have been implicated as plant defence molecules.

In wheat, ATIs represents ˜4.1% of total grain protein (Dupont et al., 2011). These proteins are encoded in the wheat genome on chromosomes 3, 6 and 7 (Singh and Skerritt, 2001) and are found in monomeric (WMAI), dimeric (WDAI) and tetrameric (WTAI) forms (Altenbach et al., 2011; Sanchez-Monge et al., 1989). Wheat-derived ATIs are known to exist as monomers as non-covalently-linked dimers (0.19 or 0.54) as tetramers (CM2 and CM3). The modern wheat genome encodes ˜17 different ATI species of ˜120 to 150 amino acids in length that vary in amino acid sequence, but share similar secondary structures (Altenbach et al., 2011).

Although several ATIs have been characterized in other cereals such as wheat, there remains a need to identify ATIs present in barley and to provide barley grain with reduced levels of ATIs to produce food and beverages for consumption by subjects with gastrointestinal sensitivities, such as NCGS.

### SUMMARY OF THE INVENTION

The present inventors have identified ATIs present in barley and have produced barley grain with reduced levels of these ATIs, relative to wild-type. This grain can be used for the production of a wide variety of foods and malt based beverages which can be consumed by subjects who suffer from a non-coeliac gastrointestinal sensitivity, such as NCGS.

In an aspect, the present invention provides a method of producing a food or malt-based beverage ingredient, or a food or a malt-based beverage, for consumption by a subject with a non-coeliac gastrointestinal sensitivity, the method comprising

(i) processing barley grain to produce processed barley grain, malt, wort, flour or wholemeal, and/or

(ii) mixing barley grain, or processed barley grain, malt, wort, flour or wholemeal produced from the grain, with at least one other food or beverage ingredient, thereby producing the food or malt-based beverage ingredient, food or malt-based beverage, wherein the barley grain has a reduced level of one or more alpha-amylase/trypsin inhibitors (ATIs) relative to grain from a corresponding wild-type barley plant.

In an embodiment, the one or more ATIs include at least one, at least two, at least three, at least four, at least five, at least six, or all of the following proteins:

(i) a protein comprising a sequence of amino acids provided as SEQ ID NO:86, or a sequence at least 95% identical thereto;

(ii) a protein comprising a sequence of amino acids provided as SEQ ID NO:87, or a sequence at least 95% identical thereto;

(iii) a protein comprising a sequence of amino acids provided as SEQ ID NO:88, or a sequence at least 95% identical thereto;

(iv) a protein comprising a sequence of amino acids provided as SEQ ID NO:89, or a sequence at least 95% identical thereto;

(v) a protein comprising a sequence of amino acids provided as SEQ ID NO:90, or a sequence at least 95% identical thereto;

(vi) a protein comprising a sequence of amino acids provided as SEQ ID NO:91, or a sequence at least 95% identical thereto; and

(vii) a protein comprising a sequence of amino acids provided as SEQ ID NO:92, or a sequence at least 95% identical thereto.

In an embodiment, the one or more ATIs include a protein comprising a sequence of amino acids provided as SEQ ID NO:86, or a sequence at least 95% identical thereto.

In an embodiment, the one or more ATIs include a protein comprising a sequence of amino acids provided as SEQ ID NO:87, or a sequence at least 95% identical thereto.

In an embodiment, the one or more ATIs include a protein comprising a sequence of amino acids provided as SEQ ID NO:88, or a sequence at least 95% identical thereto.

In an embodiment, the one or more ATIs include a protein comprising a sequence of amino acids provided as SEQ ID NO:89, or a sequence at least 95% identical thereto.

In an embodiment, the one or more ATIs include a protein comprising a sequence of amino acids provided as SEQ ID NO:90, or a sequence at least 95% identical thereto.

In an embodiment, the one or more ATIs include a protein comprising a sequence of amino acids provided as SEQ ID NO:91, or a sequence at least 95% identical thereto.

In an embodiment, the one or more ATIs include a protein comprising a sequence of amino acids provided as SEQ ID NO:92, or a sequence at least 95% identical thereto.

In a further embodiment, the barley grain has one or more of the following properties:

(i) the level of the protein comprising a sequence of amino acids provided as SEQ ID NO:86, or a sequence at least 95% identical thereto, in the barley grain is about 20% or less, about 10% or less, about 5% or less, or about 2% or less of the level in the grain from the corresponding wild-type plant;

(ii) the level of the protein comprising a sequence of amino acids provided as SEQ ID NO:87, or a sequence at least 95% identical thereto, in the barley grain is about 25% or less, about 15% or less, about 10% or less, or about 5% or less of the level in the grain from the corresponding wild-type plant;

(iii) the level of the protein comprising a sequence of amino acids provided as SEQ ID NO:88, or a sequence at least 95% identical thereto, in the barley grain is about 50% or less, about 40% or less, about 30% or less, or about 20% or less of the level in the grain from the corresponding wild-type plant;

(iv) the level of the protein comprising a sequence of amino acids provided as SEQ ID NO:89, or a sequence at least 95% identical thereto, in the barley grain is about 50% or less, about 40% or less, about 30% or less, or about 20% or less of the level in the grain from the corresponding wild-type plant;

(v) the level of the protein comprising a sequence of amino acids provided as SEQ ID NO:90, or a sequence at least 95% identical thereto, in the barley grain is about 60% or less, about 50% or less, about 40% or less, or about 35% or less of the level in the grain from the corresponding wild-type plant;

(vi) the level of the protein comprising a sequence of amino acids provided as SEQ ID NO:91, or a sequence at least 95% identical thereto, in the barley grain is about 90% or less, about 80% or less, about 75% or less, or about 70% or less of the level in the grain from the corresponding wild-type plant; and

(vii) the level of the protein comprising a sequence of amino acids provided as SEQ ID NO:92, or a sequence at least 95% identical thereto, in the barley grain is about 90% or less, about 85% or less, about 80% or less, or about 75% or less of the level in the grain from the corresponding wild-type plant.

In an embodiment, the level of the protein comprising a sequence of amino acids provided as SEQ ID NO:86, or a sequence at least 95% identical thereto, in the barley grain is about 20% or less, about 10% or less, about 5% or less, or about 2% or less of the level in the grain from the corresponding wild-type plant.

In an embodiment, the level of the protein comprising a sequence of amino acids provided as SEQ ID NO:87, or a sequence at least 95% identical thereto, in the barley grain is about 25% or less, about 15% or less, about 10% or less, or about 5% or less of the level in the grain from the corresponding wild-type plant.

In an embodiment, the level of the protein comprising a sequence of amino acids provided as SEQ ID NO:88, or a sequence at least 95% identical thereto, in the barley grain is about 50% or less, about 40% or less, about 30% or less, or about 20% or less of the level in the grain from the corresponding wild-type plant.

In an embodiment, the level of the protein comprising a sequence of amino acids provided as SEQ ID NO:89, or a sequence at least 95% identical thereto, in the barley grain is about 50% or less, about 40% or less, about 30% or less, or about 20% or less of the level in the grain from the corresponding wild-type plant.

In an embodiment, the level of the protein comprising a sequence of amino acids provided as SEQ ID NO:90, or a sequence at least 95% identical thereto, in the barley grain is about 60% or less, about 50% or less, about 40% or less, or about 35% or less of the level in the grain from the corresponding wild-type plant.

In an embodiment, the level of the protein comprising a sequence of amino acids provided as SEQ ID NO:91, or a sequence at least 95% identical thereto, in the barley grain is about 90% or less, about 80% or less, about 75% or less, or about 70% or less of the level in the grain from the corresponding wild-type plant.

In an embodiment, the level of the protein comprising a sequence of amino acids provided as SEQ ID NO:92, or a sequence at least 95% identical thereto, in the barley grain is about 90% or less, about 85% or less, about 80% or less, or about 75% or less of the level in the grain from the corresponding wild-type plant.

In an embodiment, the barley grain comprises the following proteins in a summed level which is about 95% or less, about 90% or less, or about 85% or less of the summed level in grain from a corresponding wild-type plant:

(i) a protein comprising a sequence of amino acids provided as SEQ ID NO:92, or a sequence at least 95% identical thereto;

(ii) a protein comprising a sequence of amino acids provided as SEQ ID NO:93, or a sequence at least 95% identical thereto;

(iii) a protein comprising a sequence of amino acids provided as SEQ ID NO:94, or a sequence at least 95% identical thereto;

(iv) a protein comprising a sequence of amino acids provided as SEQ ID NO:91, or a sequence at least 95% identical thereto; and

(v) a protein comprising a sequence of amino acids provided as SEQ ID NO:86, or a sequence at least 95% identical thereto.

In an embodiment, the barley grain comprises the following proteins in a summed level which is about 95% or less, about 90% or less, or about 85% or less of the summed level in grain from Sloop:

(i) a protein comprising a sequence of amino acids provided as SEQ ID NO:92, or a sequence at least 95% identical thereto;

(ii) a protein comprising a sequence of amino acids provided as SEQ ID NO:93, or a sequence at least 95% identical thereto;

(iii) a protein comprising a sequence of amino acids provided as SEQ ID NO:94, or a sequence at least 95% identical thereto;

(iv) a protein comprising a sequence of amino acids provided as SEQ ID NO:91, or a sequence at least 95% identical thereto; and

(v) a protein comprising a sequence of amino acids provided as SEQ ID NO:86, or a sequence at least 95% identical thereto.

In an embodiment, the barley grain comprises the following proteins in a summed level which is about 90% or less, about 85% or less, or about 80% or less of the summed level in grain which is homozygous for an allele at the Lys3 locus of barley which results in the barley grain lacking C-hordeins:

(i) a protein comprising a sequence of amino acids provided as SEQ ID NO:92, or a sequence at least 95% identical thereto;

(ii) a protein comprising a sequence of amino acids provided as SEQ ID NO:93, or a sequence at least 95% identical thereto;

(iii) a protein comprising a sequence of amino acids provided as SEQ ID NO:94, or a sequence at least 95% identical thereto;

(iv) a protein comprising a sequence of amino acids provided as SEQ ID NO:91, or a sequence at least 95% identical thereto; and

(v) a protein comprising a sequence of amino acids provided as SEQ ID NO:86, or a sequence at least 95% identical thereto.

In an embodiment, the barley grain comprises a summed level of the following proteins

(i) a protein comprising a sequence of amino acids provided as SEQ ID NO:92, or a sequence at least 95% identical thereto;

(ii) a protein comprising a sequence of amino acids provided as SEQ ID NO:93, or a sequence at least 95% identical thereto;

(iii) a protein comprising a sequence of amino acids provided as SEQ ID NO:94, or a sequence at least 95% identical thereto;

(iv) a protein comprising a sequence of amino acids provided as SEQ ID NO:91, or a sequence at least 95% identical thereto; and

(v) a protein comprising a sequence of amino acids provided as SEQ ID NO:86, or a sequence at least 95% identical thereto;

wherein the summed level is about 90% or less, about 80% or less, about 75% or less, or about 70% or less of the summed level in barley grain lacking C-hordeins. In an embodiment, the barley grain lacking C-hordeins is homozygous for an allele at the Lys3 locus of barley which results in the barley grain lacking C-hordeins. In an embodiment, the barley grain lacking C-hordeins is Risø 1508 grain.

In an embodiment, the barley grain has a reduced level of one or more or all of B-hordeins, C-hordeins, and D-hordeins, or any combinations thereof, relative to grain of the corresponding wild-type barley plant.

In a further embodiment, the barley grain has a level of less than 10%, less than 5% or less than 2% of a wild-type level, or is lacking, one or more than one or all of:

i) B-hordeins comprising a sequence of amino acids provided as SEQ ID NO:53,

ii) B-hordeins comprising a sequence of amino acids provided as SEQ ID NO:54,

iii) C-hordeins comprising a sequence of amino acids provided as SEQ ID NO:55, and

iv) D-hordeins comprising a sequence of amino acids provided as SEQ ID NO:56,

wherein each of the levels of less than 10%, less than 5% or less than 2% is relative to grain from a corresponding wild-type barley plant.

In an embodiment, the B-hordeins are at least B1-hordein (for example comprising an amino acid sequence provided as SEQ ID NO: 78) and B3-hordein (for example comprising an amino acid sequence provided as SEQ ID NO: 79). In a further example, the C-hordeins comprise an amino acid sequence provided as SEQ ID NO: 80. In yet another example, the D-hordeins comprise an amino acid sequence provided as SEQ ID NO: 76.

In a further embodiment, the barley grain further has a level of less than 10%, less than 5% or less than 2% of a wild-type level, or is further lacking;

i) γ-hordeins comprising a sequence of amino acids provided as SEQ ID NO:57, and/or

ii) avenin-like A proteins comprising a sequence of amino acids provided as SEQ ID NO:52, wherein each of the levels of less than 10%, less than 5% or less than 2% is relative to grain from a corresponding wild-type barley plant.

In an example, the γ-hordeins comprise an amino acid sequence provided as SEQ ID NO: 81. In yet another example, the avenin-like A proteins comprise an amino acid sequence provided as SEQ ID NO: 84.

In a further embodiment, the γ-hordeins are γ1-hordeins and γ2-hordeins. In another embodiment, the barley grain further comprises a γ3-hordein, at a level of about 60% or less when compared to the level in the corresponding wild-type barley plant, the γ3-hordein comprising amino acids whose sequence is provided as SEQ ID NO:58, such as a γ3-hordein comprising amino acids whose sequence is provided as SEQ ID NO:83.

In an embodiment, the corresponding wild-type barley plant produces grain having unmodified hordein levels. Thus, in some embodiments, the plant comprises functional B-, C-, and D-hordein genes. Examples of a wild-type barley plant include, but are not limited to, Sloop, Bomi, Baudin, Yagan, Hindmarsh, or Commander. In one embodiment, the corresponding wild-type barley plant is Sloop.

In an embodiment, the starch content of the barley grain is at least about 50% (w/w). More preferably, the starch content of the barley grain is about 50% to about 70% (w/w).

In an embodiment, the barley grain comprises about 50 ppm or less, 20 ppm or less, about 10 ppm or less, about 5 ppm or less, about 0.05 ppm to about 50 ppm, or about 0.05 ppm to about 20 ppm, about 0.05 ppm to about 10 ppm, about 0.05 ppm to about 5 ppm, about 0.1 ppm to about 5 ppm, about 3.9 ppm, or about 1.5 ppm, total hordeins.

In a further embodiment, the barley grain comprises about 1% or less, about 0.01% or less, about 0.007% or less, about 0.0027% or less, about 0.001% to about 1%, about 0.001% to about 0.01%, about 0.007%, or about 0.0027%, of the level of total hordeins when compared to grain from a corresponding wild-type barley plant.

In an embodiment, the barley grain has a reduced level of one or more of the following ATIs relative to grain from a corresponding wild-type barley plant:

(i) a protein comprising a sequence of amino acids provided as SEQ ID NO:93, or a sequence at least 95% identical thereto;

(ii) a protein comprising a sequence of amino acids provided as SEQ ID NO:94, or a sequence at least 95% identical thereto;

(iii) a protein comprising a sequence of amino acids provided as SEQ ID NO:96, or a sequence at least 95% identical thereto; and

(iv) a protein comprising a sequence of amino acids provided as SEQ ID NO:97, or a sequence at least 95% identical thereto.

In an embodiment, the barley grain has a reduced level of a protein comprising a sequence of amino acids provided as SEQ ID NO:93, or a sequence at least 95% identical thereto, relative to grain from a corresponding wild-type barley plant.

In an embodiment, the barley grain has a reduced level of a protein comprising a sequence of amino acids provided as SEQ ID NO:93, or a sequence at least 95% identical thereto, relative to grain from a corresponding wild-type barley plant.

In an embodiment, the barley grain has a reduced level of a protein comprising a sequence of amino acids provided as SEQ ID NO:94, or a sequence at least 95% identical thereto, relative to grain from a corresponding wild-type barley plant.

In an embodiment, the barley grain has a reduced level of a protein comprising a sequence of amino acids provided as SEQ ID NO:96, or a sequence at least 95% identical thereto, relative to grain from a corresponding wild-type barley plant.

In an embodiment, the barley grain has a reduced level of a protein comprising a sequence of amino acids provided as SEQ ID NO:97, or a sequence at least 95% identical thereto, relative to grain from a corresponding wild-type barley plant.

In an embodiment, the average weight of the barley grain is at least about 35 mg, at least about 39 mg, at least about 41 mg, at least about 47 mg, about 35 mg to about 60 mg, about 40 mg to about 60 mg, about 45 mg to about 60 mg, about 39.1 mg, about 41.8 mg or about 47.2 mg.

In an embodiment, at least about 80%, at least about 90%, at least about 95%, about 80% to about 98%, or about 80% to about 93%, of the barley grain do not pass through a 2.8 mm sieve.

In an embodiment, the barley grain is from a plant which has a harvest index of at least 40%, about 40% to about 60%, about 40% to about 55%, or about 40% to about 50%.

In an embodiment, the barley grain has a length to thickness ratio of less than about 5, less than about 4, less than about 3.8, about 2 to about 5, or about 2.5 to about 3.8.

In an embodiment, the barley grain is homozygous for an allele of the Hor2 locus where most or all of the B-hordein encoding genes have been deleted, or wherein the processed barley grain, malt, wort, flour or wholemeal produced from said grain comprises DNA which comprises the allele of the Hor2 locus where most or all of the B-hordein encoding genes have been deleted.

In an embodiment, the barley grain is homozygous for a null allele of the gene encoding D-hordein at the Hor3 locus, or wherein the processed barley grain, malt, wort, flour or wholemeal produced from said grain comprises DNA which comprises the null allele of the gene encoding D-hordein, the null allele preferably comprising a stop codon, splice site mutation, frame-shift mutation, insertion, deletion or encoding a truncated D-hordein, or where most or all of the D-hordein encoding gene has been deleted.

In an embodiment, the truncated D-hordein has a stop codon at the triplet encoding amino acid number 150.

In an embodiment, the barley grain is homozygous for an allele at the Lys3 locus of barley which results in the barley grain lacking C-hordeins, or wherein the processed barley grain, malt, wort, flour or wholemeal produced from said grain comprises DNA which comprises the allele at the Lys3 locus.

In an embodiment, the barley grain:
is homozygous for an allele of the Hor2 locus where most or all of the B-hordein encoding genes have been deleted, or wherein the processed barley grain, malt, wort, flour or wholemeal produced from said grain comprises DNA which comprises the allele of the Hor2 locus where most or all of the B-hordein encoding genes have been deleted,
is homozygous for a null allele of the gene encoding D-hordein at the Hor3 locus, or wherein the processed barley grain, malt, wort, flour or wholemeal produced from said grain comprises DNA which comprises the null allele of the gene encoding D-hordein, and
lacks C-hordeins comprising a sequence of amino acids provided as SEQ ID NO:55.

In an embodiment, the barley grain is from a plant described in WO 2014/197943 or WO 2009/021285.

In an embodiment, the barley grain comprises one or more of the following proteins at a level which is higher than in grain from a corresponding wild-type barley plant:

(i) a protein comprising a sequence of amino acids provided as SEQ ID NO:95, or a sequence at least 95% identical thereto;

(ii) a protein comprising a sequence of amino acids provided as SEQ ID NO:94, or a sequence at least 95% identical thereto;

(iii) a protein comprising a sequence of amino acids provided as SEQ ID NO:96, or a sequence at least 95% identical thereto; and

(iv) a protein comprising a sequence of amino acids provided as SEQ ID NO:97, or a sequence at least 95% identical thereto.

In an embodiment, the barley grain comprises a protein comprising a sequence of amino acids provided as SEQ ID NO:95, or a sequence at least 95% identical thereto, at a level which is higher than in grain from a corresponding wild-type barley plant.

In an embodiment, the barley grain comprises a protein comprising a sequence of amino acids provided as SEQ ID NO:94, or a sequence at least 95% identical thereto, at a level which is higher than in grain from a corresponding wild-type barley plant.

In an embodiment, the barley grain comprises a protein comprising a sequence of amino acids provided as SEQ ID NO:96, or a sequence at least 95% identical thereto, at a level which is higher than in grain from a corresponding wild-type barley plant.

In an embodiment, the barley grain comprises a protein comprising a sequence of amino acids provided as SEQ ID NO:97, or a sequence at least 95% identical thereto, at a level which is higher than in grain from a corresponding wild-type barley plant.

In a further embodiment, the barley grain has one or more of the following properties:

(i) the level of the protein comprising a sequence of amino acids provided as SEQ ID NO:95, or a sequence at least 95% identical thereto, in the barley grain is at least about 2-fold higher, at least about 5-fold higher, at least about 10-fold higher, or at least about 20-fold higher than the level in the grain from the corresponding wild-type plant;

(ii) the level of the protein comprising a sequence of amino acids provided as SEQ ID NO:94, or a sequence at least 95% identical thereto, in the barley grain is at least about 1.4-fold higher, at least about 1.6-fold higher, at least about 1.8-fold higher, or at least about 1.9-fold higher than the level in the grain from the corresponding wild-type plant;

(iii) the level of the protein comprising a sequence of amino acids provided as SEQ ID NO:96, or a sequence at least 95% identical thereto, in the barley grain is at least about 1.3-fold higher, at least about 1.5-fold higher, at least about 1.7-fold higher, or at least about 1.8-fold higher than the level in the grain from the corresponding wild-type plant; and

(iv) the level of the protein comprising a sequence of amino acids provided as SEQ ID NO:97, or a sequence at least 95% identical thereto, in the barley grain is at least about 1.7-fold higher, at least about 1.9-fold higher, at least about 2.1-fold higher, or at least about 2.3-fold higher than the level in the grain from the corresponding wild-type plant.

In an embodiment, the level of the protein comprising a sequence of amino acids provided as SEQ ID NO:95, or a sequence at least 95% identical thereto, in the barley grain is at least about 2-fold higher, at least about 5-fold higher, at least about 10-fold higher, or at least about 20-fold higher than the level in the grain from the corresponding wild-type plant.

In an embodiment, the level of the protein comprising a sequence of amino acids provided as SEQ ID NO:94, or a sequence at least 95% identical thereto, in the barley grain is at least about 1.4-fold higher, at least about 1.6-fold higher, at least about 1.8-fold higher, or at least about 1.9-fold higher than the level in the grain from the corresponding wild-type plant.

In an embodiment, the level of the protein comprising a sequence of amino acids provided as SEQ ID NO:96, or a sequence at least 95% identical thereto, in the barley grain is at least about 1.3-fold higher, at least about 1.5-fold higher, at least about 1.7-fold higher, or at least about 1.8-fold higher than the level in the grain from the corresponding wild-type plant.

In an embodiment, the level of the protein comprising a sequence of amino acids provided as SEQ ID NO:97, or a sequence at least 95% identical thereto, in the barley grain is at least about 1.7-fold higher, at least about 1.9-fold higher, at least about 2.1-fold higher, or at least about 2.3-fold higher than the level in the grain from the corresponding wild-type plant.

In an embodiment, the average grain weight is at least 1.05 fold, at least 1.1 fold, or 1.05 to 1.3 fold, higher than a grain which is

i) homozygous for an allele of the Hor2 locus where most or all of the B-hordein encoding genes have been deleted,

ii) homozygous for an allele at the Lys3 locus of barley which results in the barley grain lacking C hordeins, and

iii) homozygous for a wild-type allele of D hordein encoding a full-length protein.

In an embodiment, the barley grain is from a plant which has a grain yield which is least 1.20 fold, or at least 1.35 fold, or 1.2 to 1.5 fold, or 1.2 to 2.0 fold higher than the grain yield from a plant which is

i) homozygous for an allele of the Hor2 locus where most or all of the B-hordein encoding genes have been deleted,

ii) homozygous for an allele at the Lys3 locus of barley which results in the barley grain lacking C hordeins, and

iii) homozygous for a wild-type allele of D hordein encoding a full-length protein.

In an embodiment, at least about 50% of the genome of the barley grain is identical to the genome of a barley cultivar Sloop, Hindmarsh, Oxford or Maratime.

In an embodiment, the barley grain is from a plant comprising one or more genetic variations which reduce the level of the one or more ATIs in the barley grain relative to grain from the corresponding wild-type barley plant.

In some embodiments, the one or more genetic variations include a mutation (e.g., a substitution, a deletion, or an insertion). Thus, in an embodiment, the barley grain is from a non-transgenic plant.

In some embodiments, the one or more genetic variations include a transgene. Thus, in an embodiment, the barley grain is from a transgenic plant.

In a further embodiment, the plant comprises a transgene which encodes a polynucleotide which down-regulates the production of at least one ATI in the barley grain. Preferably, the polynucleotide of this embodiment is an antisense polynucleotide, a sense polynucleotide, a catalytic polynucleotide, an artificial microRNA or a duplex RNA molecule which down-regulates expression of one or more genes encoding ATIs.

In an embodiment, the plant comprises a transgene which encodes a polynucleotide which down-regulates the production of at least one hordein in the barley grain. In an embodiment, the plant comprises a transgene which encodes a polynucleotide which down-regulates the production of at least one hordein and/or at least one ATI in the barley grain.

In an embodiment, the method comprises producing processed barley grain from the barley grain. In an embodiment, the processed barley grain is dehulled barley or pearl barley. In an embodiment, the processed barley grain is dehulled barley. In an embodiment, the processed barley grain is pearl barley.

In an embodiment, the method comprises producing flour or wholemeal from the barley grain.

In an embodiment, the method comprises producing malt from the barley grain.

In an embodiment, the malt-based beverage is beer and the method comprises germinating the barley grain or cracked grain derived therefrom. In a further embodiment, the method comprises fractionating dried germinated grain into two or more of an endosperm fraction, an endothelial layer fraction, a husk fraction, an acrospire fraction, and a malt rootlets fraction; and combining and blending predetermined amounts of two or more of the fractions.

In an embodiment, at least about 50% of the barley grain germinates within 3 days following imbibition.

In an embodiment, the food ingredient or malt-based beverage ingredient is processed barley grain, flour, starch, malt, or wort, or wherein the food is processed barley grain, soup, stew, gruel, leavened or unleavened breads, pasta, noodles, breakfast cereals, snack foods, cakes, pastries or foods containing flour-based sauces.

In an embodiment, the food ingredient is processed barley grain. In an embodiment, the food is processed barley grain.

In an embodiment, the malt-based beverage is beer or whiskey.

In an embodiment, following consumption of the food or malt-based beverage by the subject, at least one symptom of the subject's gastrointestinal sensitivity is not developed or worsened.

In an embodiment, the subject has non-coeliac gluten sensitivity (NCGS).

In an embodiment, the subject has diabetes.

In an embodiment, the subject is a mammal. In a further embodiment, the subject is a human.

In an embodiment, the subject does not have coeliac disease. In an embodiment, the subject does not have a wheat allergy.

In some embodiments, the wild-type barley plant is Sloop, Hindmarsh, or Commander.

In another aspect, the present invention provides a product produced according to the method described herein.

In an embodiment, the product is a food ingredient, malt-based beverage ingredient, food product or malt-based beverage product.

In an embodiment, the malt-based beverage product is beer or whiskey.

In a further embodiment, the beer comprises at least about 2%, at least about 3%, at least about 4%, or at least about 5%, ethanol.

In an embodiment, the malt-based beverage ingredient is malt or wort.

In an embodiment, the food is processed barley grain, soup, stew, gruel, leavened or unleavened bread, pasta, noodles, breakfast cereal, snack food, cake, pastry or a food containing a flour-based sauce.

In an embodiment, the food ingredient is processed barley grain, flour or wholemeal.

In another aspect, the present invention provides a packaged product comprising (i) the product described herein, and

(ii) packaging which indicates that the product is suitable for consumption by a subject with a non-coeliac gastrointestinal sensitivity. For example, the packaging may include a statement that the product is “suitable for people with sensitive stomachs” or “suitable for people with a gluten intolerance” or “suitable for people with NCGS”.

In another aspect, the present invention provides a packaged product comprising

(i) a product comprising a food or malt-based beverage ingredient, or a food or a malt-based beverage produced from barley grain having a reduced level of one or more ATIs relative to grain from a corresponding wild-type barley plant, and

(ii) packaging which indicates that the product is suitable for consumption by a subject with a non-coeliac gastrointestinal sensitivity.

In another aspect, the present invention provides a method of feeding a subject with a non-coeliac gastrointestinal sensitivity, the method comprising providing the subject with a food or malt-based beverage produced from barley grain which has a reduced level of one or more alpha-amylase/trypsin inhibitors (ATIs) relative to grain from a corresponding wild-type barley plant.

In another aspect, the present invention provides method of reducing the incidence or severity of a non-coeliac gastrointestinal sensitivity in a subject, the method comprising feeding the subject a food or malt-based beverage produced from barley grain which has a reduced level of one or more alpha-amylase/trypsin inhibitors (ATIs) relative to grain from a corresponding wild-type barley plant, wherein the reduction of the incidence or severity of the non-coeliac gastrointestinal sensitivity is relative to when the subject is fed the same amount of a corresponding food or malt-based beverage produced from grain from a corresponding wild-type barley plant.

In another aspect, the present invention provides use of barley grain which has a reduced level of one or more alpha-amylase/trypsin inhibitors (ATIs) relative to grain from a corresponding wild-type barley plant in the manufacture of a food or malt-based beverage for reducing the incidence or severity of a non-coeliac gastrointestinal sensitivity in a subject, wherein the reduction of the incidence or severity of the non-coeliac gastrointestinal sensitivity is relative to when the subject is fed the same amount of a corresponding food or malt-based beverage produced from grain from a corresponding wild-type barley plant.

In another aspect, the present invention provides use of barley grain which has a reduced level of one or more alpha-amylase/trypsin inhibitors (ATIs) relative to grain from a corresponding wild-type barley plant in the manufacture of a food or malt-based beverage for consumption by a subject with a non-coeliac gastrointestinal sensitivity.

In another aspect, the present invention provides a food or malt-based beverage produced from barley grain which has a reduced level of one or more alpha-amylase/trypsin inhibitors (ATIs) relative to grain from a corresponding wild-type barley plant, for use in reducing the incidence or severity of a non-coeliac gastrointestinal sensitivity in a subject, wherein the reduction of the incidence or severity of the non-coeliac gastrointestinal sensitivity is relative to when the subject is fed the same amount of a corresponding food or malt-based beverage produced from grain from a corresponding wild-type barley plant.

In another aspect, the present invention provides a barley grain which is homozygous for an allele at the Lys3 locus of barley which results in the barley grain lacking C-hordeins, wherein the barley grain has a reduced level of one or more of the following alpha-amylase/trypsin inhibitors (ATIs) relative to grain from a corresponding wild-type barley plant:

(i) a protein comprising a sequence of amino acids provided as SEQ ID NO:93;

(ii) a protein comprising a sequence of amino acids provided as SEQ ID NO:94;

(iii) a protein comprising a sequence of amino acids provided as SEQ ID NO:96; and

(iv) a protein comprising a sequence of amino acids provided as SEQ ID NO:97. Such grain can be produced, for example, by further mutagenizing the hordein mutant grain described in Example 2 using routine methods known in the art, e.g., the methods described in Example 10. Resulting mutants can be screened for expression of the proteins comprising sequences provided as SEQ ID NOs: 93, 94, 96, or 97 to identify mutant grain having a reduced level of expression of one of these proteins.

In a further embodiment:

(i) the barley grain is homozygous for an allele of the Hor2 locus where most or all of the B-hordein encoding genes have been deleted, and/or

(ii) the barley grain is homozygous for a null allele of the gene encoding D-hordein at the Hor3 locus.

In another aspect, the present invention provides a barley grain which has a reduced level of one or more alpha-amylase/trypsin inhibitors (ATIs) relative to grain from a corresponding wild-type barley plant. In an embodiment, the barley grain has a reduced level of one or more of the following alpha-amylase/trypsin inhibitors (ATIs) relative to grain from a corresponding wild-type barley plant:

(i) a protein comprising a sequence of amino acids provided as SEQ ID NO:93;

(ii) a protein comprising a sequence of amino acids provided as SEQ ID NO:94;

(iii) a protein comprising a sequence of amino acids provided as SEQ ID NO:96; and

(iv) a protein comprising a sequence of amino acids provided as SEQ ID NO:97.

In an embodiment, the barley grain is dehulled barley or peal barley grain.

In another aspect, the present invention provides a barley plant which produces grain of the invention.

In another aspect, the present invention provides a method of producing barley grain, the method comprising;

a) growing a barley plant of the invention,

b) harvesting the barley grain, and

c) optionally processing the barley grain.

In an embodiment, the method comprises growing at least 10,000 plants in a field in an area of at least one hectare.

In another aspect, the present invention provides a method of producing processed barley grain, flour, wholemeal, starch, malt, wort or other product obtained from grain, the method comprising;

a) obtaining barley grain of the invention, and

b) processing the barley grain to produce the processed barley grain, flour, wholemeal, starch, malt, wort or other product.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Determination of hordein content of ULG3.0 lines by MRM MS by peak area, and indicating percentage relative to Sloop (100%).

FIG. 2. Determination of hordein content of ULG3.0 lines by MRM MS by peak area, and indicating percentage relative to Sloop (100%).

FIG. 3. Detection of hordeins and hordein-like proteins by MRM MS in flour from ULG3.0 (T2-4-8) and ULG3.2 candidate lines, compared to wild-type varieties Sloop, Baudin, Commander and Hindmarsh. Each graph shows the mean+/−SD for the summed peak area (3 MRM transitions) for a representative peptide from each hordein family. In each case the peptide (sequence as listed in graph) maps to either an avenin-like A protein, B1-, B3-, C-, D-, γ1- or γ3-hordein. The Uniprot accession number is given in the legend (e.g. F2EGD5 for first example).

FIG. 4. Relative quantitation of two exemplar peptides from four ATIs across three wild-type barley cultivars: Hindmarsh, Sloop and Commander. For ease of comparison between peptides that generated different responses in mass spectrometry, all data were converted to a percentage of the average peak area for all three barley lines used in this experiment. Data is presented as the mean±S.D. (n=4) with two peptides from each ATI depicted.

FIG. 5. Identification of ATIs from barley varieties. (a) Phylogenetic tree showing the ATI protein clusters; alpha-amylase/trypsin inhibitor CMd (ATI CMd), alpha-amylase/trypsin inhibitor CMb (ATI CMb), alpha-amylase/trypsin inhibitor CMa (ATI CMa), trypsin inhibitor CMc (TI CMc), trypsin inhibitor CMe (TI CMe), uncharacterized protein (similar to Baker's asthma allergen BDP), thaumatin-like protein 8 (TLP8), globulin, alpha-amylase inhibitor BDAI (ATI BDAI-1), alpha-amylase inhibitor BMAI (ATI BMAI-1), predicted protein (ATI-like), alpha-amylase/subtilisin inhibitor (AASI); (b) Schematics showing the conserved domains for the proteins. AAI—alpha-amylase inhibitor, STI—soybean trypsin inhibitor; and THN—thaumatin family; (c) Multiple sequence alignment showing the conservation of cysteines (≥50%) in ATIs. (*) shows the cysteine conservation in ATIs,

FIG. 6. Relative quantitation of two example peptides from each of six ATIs across the ULG mutant barley lines. Graphs show percentage (based on MRM peak area) relative to cv Sloop (WT). (a) ATI CMa (A0A287W0A8): EYVAQQTCGVSIAGSPVSTEPGDTPK (SEQ ID NO:120) and SHPDWSVLK (SEQ ID NO:121); (b) ATI CMb (P32936): DYVEQQACR (SEQ ID NO:107) and EVQMDFVR (SEQ ID NO:108); (c) TI CMc (P34951): ELAGISSNCR (SEQ ID NO:113) and FYVASQTCGAVPLLPIEVMK (SEQ ID NO:114); (d) ATI CMd (P11643): DYVLQQTCAVFTPGSK (SEQ ID NO:98) and LLVAPGQCNLATIHNVR (SEQ ID NO:99); (e) TI CMe (P01086): DSPNCPR (SEQ ID NO:116) and LTSDMK (SEQ ID NO:117); (f) Uncharacterized protein (similar to Baker's asthma allergen BDP) (AOA287JQN1): ELSDLPESCR (SEQ ID NO:125) and SIPINPLPACR (SEQ ID NO:127). Statistical significance was calculated between WTg and BCD-null lines based on a two-sided Welch's t-test; data shown as mean±S.D (n=4), ns=not significant, *p<0.05 and **<0.001.

FIG. 7. Summary statistics of ATI peptides from CMa-e proteins across WT-g and BCD-null lines. Box plot comparing average CMa-e ATIs from three lines measured by LC-MRM-MS in quadruplicate. The y-axis indicates the linear value of CMa-e ATIs quantified by LC-MRM-MS and presented as % relative to average (WT-g extracts). The horizontal dotted line shows the mean value and each whisker extends to the most extreme value. The mean value for WT-g=100, C-null=110 and BCD-null=83.

FIG. 8. Relative quantitation of two example peptides from six ATIs across the ULG mutant barley lines. Graphs show the percentage (based on MRM peak area) relative to cv Sloop (WT). (a) Thaumatin (F2DNP3): TGCSFDGAGNGR (SEQ ID NO:139) and VITPACPNELR (SEQ ID NO:140); (b) Globulin (F2EJF0): DYEQSMPPLR (SEQ ID NO:149) and QILEHQLTGR (SEQ ID NO:150); (c) AAI BMAI-1 (MOUYA9): SQCAGGQVVESIQK (SEQ ID NO:128) and ATVAEVFPGCR (SEQ ID NO:129); (d) AAI BDAI-1 (P13691): CGDLGSMLR (SEQ ID NO:101) and LLVAGVPALCNVPIPNEAAGTR (SEQ ID NO:104); (e) alpha-amylase/subtilisin inhibitor-like (F2E994): DLVLLDYAGR (SEQ ID NO:132) and EPLVVVFK (SEQ ID NO:133). (f) Bifunctional alpha-amylase/subtilisin inhibitor (F2E8J4): SADPPPVHDTDGHELR (SEQ ID NO:147) and YSGAEVHEYK (SEQ ID NO:148). Statistical significance was calculated between WTg and BCD-null lines based on a two-sided Welch's t-test; data shown as mean±S.D (n=4), ns=not significant, *p<0.05, **<0.001, ***<p<0.0001.

FIG. 9. Identification of ATI homologues between barley and wheat. (a) Phylogenetic tree; TI—trypsin inhibitor and ATI—amylase trypsin inhibitor; (b) multiple sequence alignment. Despite the low sequence homology between wheat and barley ATIs, ATIs from barley and wheat have conserved AAI domains which are implicated in amylase/trypsin inhibition and thought to have a role in NCGS pathophysiology by triggering the TLR4 pathway.

### KEY TO THE SEQUENCE LISTING

SEQ ID NOs: 1 and 4—Wheat alpha-gliadin peptides.

SEQ ID NOs 2, 3, 6 to 11, 16 to 59 and 85—Barley hordein peptides.

SEQ ID NO: 5—Wheat avenin-like A peptide.

SEQ ID NOs 12 to 15—Rye prolamin peptides.

SEQ ID NOs 60 to 71—Oligonucleotide primers.

SEQ ID NO: 72—Genomic region encoding barley cv. Sloop D-hordein.

SEQ ID NO: 73—Genomic region encoding barley cv. Ethiopia R118 D-hordein (null).

SEQ ID NO: 74—Barley cv. Sloop D-hordein.

SEQ ID NO: 75—Barley cv. Ethiopia R1118 D-hordein.

SEQ ID NO: 74—Barley cv. Sloop D-hordein.

SEQ ID NO: 75—Barley cv. Ethiopia R118 D-hordein.

SEQ ID NO: 76—Open reading frame encoding barley cv. Sloop D hordein.

SEQ ID NO: 77—Open reading frame encoding barley cv. Ethiopia R118 D hordein.

SEQ ID NO: 78—Example of wild-type barley B1-hordein (Accession: Q40020).

SEQ ID NO: 79—Example of wild-type barley B3-hordein (Accession: Q4G3S1).

SEQ ID NO: 80—Example of wild-type barley C-hordein (Accession: Q40055).

SEQ ID NO: 81—Example of wild-type barley γ1-hordein (Accession: P17990).

SEQ ID NO: 82—Example of wild-type barley γ2-hordein (Accession: Q701B4).

SEQ ID NO: 83—Example of wild-type barley γ3-hordein (Accession: P80198).

SEQ ID NO: 84—Example of wild-type barley avenin-like A protein (Accession: F2EGD5).

SEQ ID NO: 86—Amino acid sequence of trypsin inhibitor CMe from barley, accession number P01086.

SEQ ID NO: 87—Amino acid sequence of a predicted protein from barley, accession number F2EJF0.

SEQ ID NO: 88—Amino acid sequence of alpha-amylase inhibitor BMAI-1 from barley, accession number MOUYA9.

SEQ ID NO: 89—Amino acid sequence of alpha-amylase inhibitor BDAI-1 from barley, accession number P13691.

SEQ ID NO: 90—Amino acid sequence of an AAI domain-containing protein from barley, accession number AOA287JQN1.

SEQ ID NO: 91—Amino acid sequence of alpha-amylase/trypsin inhibitor CMd from barley, accession number P11643.

SEQ ID NO: 92—Amino acid sequence of alpha-amylase/trypsin inhibitor CMa, accession number A0A287W0A8.

SEQ ID NO: 93—Amino acid sequence of trypsin inhibitor CMb from barley, accession number P32936.

SEQ ID NO: 94—Amino acid sequence of trypsin inhibitor CMc from barley, accession number P34951.

SEQ ID NO: 95—Amino acid sequence of a thaumatin-like protein from barley, accession number F2DNP3.

SEQ ID NO: 96—Amino acid sequence of a predicted protein from barley, accession number F2E994.

SEQ ID NO: 97—Amino acid sequence of a predicted protein from barley, accession number F2E8J4.

SEQ ID NOs: 98 to 151—Amino acid sequences of tryptic peptides for quantifying ATIs from barley

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in plant breeding, food technology, cell culture, molecular genetics, immunology, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

A used herein, the term “barley” refers to any species of the Genus Hordeum, including progenitors thereof, as well as progeny thereof produced by crosses with other species. A preferred form of barley is the species Hordeum vulgare. The grains of most cultivars of barley grown commercially in the world today have covered (hulled) caryopses in which the so-called hull, which is the outer lemma and inner palea, is joined to the pericarp epidermis at maturity. Other cultivars, termed hull-less or naked barleys, are free threshing and the hulls are easily removed in the threshing process. Naked barley grains are preferred for human consumption, although hulled grain can be used after pearling, whereas hulled barley grain is preferred for the brewing industry and for animal feed. The hull-less grain trait is controlled by a single, recessive gene designated nud located on the long arm of chromosome 7H (Kikuchi et al., 2003).

The phrase “non-coeliac gastrointestinal sensitivity” refers to any sensitivity of the gastrointestinal tract that is not coeliac disease. In some embodiments, the non-coeliac gastrointestinal sensitivity is associated with an intolerance to a particular food and thus symptoms occur due to consumption of those foods, e.g., cereals. In an embodiment, the non-coeliac gastrointestinal sensitivity is associated with cereal consumption. In some embodiments, the non-coeliac gastrointestinal sensitivity is associated with an immune response in the subject, for example an immune response that is illicited by a particular food. In some embodiments, the non-coeliac gastrointestinal sensitivity is associated with activation of the innate immune system. In some embodiments, the non-coeliac gastrointestinal sensitivity is associated with inflammation. In some embodiments, the non-coeliac gastrointestinal sensitivity is associated with systemic inflammation. Symptoms of non-coeliac gastrointestinal sensitivities include recurring gas, bloating, nausea, vomiting, constipation, or diarrhea. Examples of non-coeliac gastrointestinal sensitivities include non-coeliac gluten sensitivity (NCGS), non-coeliac wheat sensitivity, and diabetes.

As used herein, the phrase “non-coeliac gluten sensitivity” or “NCGS” refers to a syndrome that is induced by consumption of gluten-containing food or drink and is associated with both intestinal and/or extraintestinal symptoms that improve once gluten-containing foods are removed from the diet, and wherein coeliac disease has been excluded. The pathogenesis of NCGS is not yet well understood, but the activation of the innate immune system, the direct cytotoxic effects of gluten-containing foods are implicated. In particular, activation of the innate immune system by ATIs is known to be associated with NCGS. Gastrointestinal symptoms of NCGS include abdominal pain, bloating, diarrhea, constipation, nausea, aerophagia, flatulence, gastroesophageal reflux, and aphthous stomatitis. Extraintestinal symptoms of NCGS include headache, migraine, “foggy mind”, fatigue, fibromyalgia, joint and muscle pain, leg or arm numbness, tingling of the extremities, dermatitis, atopic disorders such as asthma, rhinitis, other allergies, as well as a wide range of neurological and psychiatric disorders.

As used herein, the term “lacking” as used herein in the context of a recited substance means that the substance is absent from the barley grain, or a product derived therefrom, of the invention, or that the substance is not detected in the grain or product of the invention when assays for the substance are performed using a method known in the art. That is, the substance may be present at a level that is insufficient for detection, or within the standard error for the assay for that substance. For example, in the context of a recited hordein, the term “lacking” means that the specific hordein is not detected in an assay such as, for example, an MRM MS assay, an ELISA assay or a 2D-gel electrophoresis assay, such as exemplified herein. The substance that is lacking may be undetected in one type of assay or in multiple types of assays. It would be appreciated that the substance that is said to be lacking in the grain or product of the invention is present, as readily determined by an assay known in the art, in the corresponding wild-type grain or product.

As used herein, the term “null allele” in relation to a gene encoding a protein refers to any such allele which does not encode the functional protein. A null allele may comprise a deletion or a truncation of the gene encoding the protein, for example.

The terms “seed” and “grain” are used interchangeably herein. “Grain” generally refers to mature, harvested grain but can also refer to grain after processing such as, for example, milling or polishing, where most of the grain stays intact, or after imbibition or germination, according to the context. Mature grain commonly has a moisture content of less than about 18-20%. Wild-type barley grain (whole grain) generally contains 9-12% protein, and about 30-50% of this is prolamin, typically 35%, so wild-type barley grain has about 3-4% prolamin by weight. Prolamins are found almost exclusively in the endosperm, which is about 70% of the wholegrain weight.

As used herein, the term “havest index” refers to the weight of the harvested grain as a percentage of the total weight of the plant.

As used herein, the term “corresponding wild-type” barley plant refers to a plant which comprises at least 50%, more preferably at least 75%, more preferably at least 95%, more preferably at least 97%, more preferably at least 99%, and even more preferably 99.5% of the genotype of a plant of the invention, but produces grain with unmodified ATI levels. In one embodiment, the “corresponding wild-type” barley plant is a cultivar used in plant breeding experiments to introduce genetic variants that result in reduced ATI production in the grain. In another embodiment, the “corresponding wild-type” barley plant is a parental cultivar into which a transgene has been introduced which reduces ATI production in the grain. In an embodiment, the corresponding wild-type barley plant produces grain having unmodified hordein levels. Thus, in some embodiments, the plant comprises one or more or all of functional B-, C-, and D-hordein genes. In a further embodiment, the “corresponding wild-type” barley plant is a cultivar that is used at the date of filing for the commercial production of barley grain such as, but not limited to, Bomi, Sloop, Carlsberg II, K8, L1, Vlamingh, Stirling, Hamelin, Schooner, Baudin, Commander, Gairdner, Buloke, WI3586-1747, WI3416, Flagship, Cowabbie, Franklin, SloopSA, SloopVic, Quasar, VB9104, Grimmett, Cameo*Arupo 31-04, Prior, Schooner, Unicorn, Harrington, Torrens, Galleon, Morex, Dhow, Capstan, Fleet, Keel, Maritime, Yarra, Dash, Doolup, Fitzgerald, Molloy, Mundah, Oxford, Onslow, Skiff, Unicorn, Yagan, Chebec, Hindmarsh, Chariot, Diamant, Korál, Rubin, Bonus, Zenit, Akcent, Forum, Amulet, Tolar, Hens, Maresi, Landora, Caruso, Miralix, Wikingett Brise, Caruso, Potter, Pasadena, Annabell, Maud, Extract, Saloon, Prestige, Astoria, Elo, Cork, Extract, Laura.

As used herein, “average weight of the grain” is preferably determined by obtaining at least 25, at least 50 or at least 100, more preferably about 100, individual grains from a plant (or genetically identical plants grown under the same conditions) and determining the average weight of the grain.

As used herein, the term “malt” is used to refer to barley malt, “flour” to refer to barley flour, “wholemeal” to refer to barley wholemeal, and “beer” to refer to beer which is produced using barley as its main ingredient providing fermentable carbohydrate, except where the malt, flour, wholemeal or beer is explicitly stated to come from a source other than barley. As used herein, “wort” refers to the liquid extracted from the mashing process during the brewing of beer or whiskey. Wort contains the sugars that will be fermented by the brewing yeast to produce alcohol. More specifically, a source of malt, wort, flour, beer, wholemeal, food product etc of the invention is from the processing (for example, milling and/or fermentation) of barley grain. The grain, malt, wort, flour, wholemeal or beer of the invention may be mixed or blended with grain, malt, wort, flour, wholemeal or beer which is not derived from barley. These terms include malt, wort, flour, beer, wholemeal, food product etc produced from a mixture of grains including barley. In a preferred embodiment, at least 10% or at least 50% of the grain used to produce the malt, wort, flour, beer, wholemeal, food product etc is barley grain.

“Nucleic acid molecule” refers to a polynucleotide such as, for example, DNA, RNA or oligonucleotides. It may be DNA or RNA of genomic or synthetic origin, double-stranded or single-stranded, and combined with carbohydrate, lipids, protein, or other materials to perform a particular activity defined herein.

“Operably linked” as used herein refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence, such as a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

As used herein, the term “gene” is to be taken in its broadest context and includes the deoxyribonucleotide sequences comprising the protein coding region of a structural gene and including sequences located adjacent to the coding region on both the 5′ and 3′ ends for a distance of at least about 2 kb on either end and which are involved in expression of the gene. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, the term “other food or beverage ingredient” refers to any substance suitable for consumption by an animal, preferably any substance suitable for consumption by a human, when provided as part of a food or beverage. Examples include, but are not limited to, grain from other plant species, sugar, etc, but excluding water.

The term “plant” as used herein as a noun refers to a whole plant such as, for example, a plant growing in a field for commercial barley production. A “plant part” refers to plant vegetative structures (for example, leaves, stems), roots, floral organs/structures, seed (including embryo, endosperm, and seed coat), plant tissue (for example, vascular tissue, ground tissue, and the like), cells, starch granules or progeny of the same.

Barley plants described herein may have one or more genetic variations which result in reduced levels of at least one ATI. As used herein, the term “genetic variation”, or similar phrases such as “genetic modification”, refers to any alteration of a nucleic acid of a barley plant not present in a corresponding wild-type plant, for example a deletion of a gene(s) or part thereof, or a mutation which reduces gene transcription, or a transgene that expresses or RNA that directly or indirectly reduces levels of at least one ATI. The genetic variation may be introduced into the plant, or an ancestor thereof, by a variety of techniques such as chemical mutagenesis, gene editing or insertion of a transgene. In some embodiments, the one or more genetic variations are man made. Examples of genetic variations are present in Risø 56, Risø 527 and Risø 1508 and Ethiopia R118. Hence, such plants may be used as parental plants to produce barley plants with reduced ATI levels. Such a plant may result from the progeny from a cross between any of these barley mutants. For example, a barley plant having reduced ATI levels may be the progeny from a cross between Risø 56 and Risø 1508 comprising the hor2 and lys3 mutations present in these lines. In an embodiment, the plant encodes γ3-hordein comprising amino acids whose sequence is provided as SEQ ID NO:58, such as a γ3-hordein comprising amino acids whose sequence is provided as SEQ ID NO:83. For instance, the plant may have a functional wild-type γ3-hordein gene such as the γ3-hordein gene of barley cultivar Bomi, Sloop, Baudin, Yagan, Hindmarsh, or Commander.

A “transgenic plant”, or variations thereof, generally refers to a plant that contains a gene construct (“transgene”) not found in a wild-type plant of the same species, variety or cultivar. A “transgene” as referred to herein has the normal meaning in the art of biotechnology and includes a genetic sequence which has been produced or altered by recombinant DNA or RNA technology and which has been introduced into the plant cell. The transgene may include genetic sequences derived from a plant cell. Typically, the transgene has been introduced into the plant, or one of its ancestors, by human manipulation such as, for example, by transformation but any method can be used as one of skill in the art recognizes. In contrast, a “non-transgenic plant” generally refers to a plant which does not comprise an artificially introduced transgene. For example, a non-transgenic plant may comprise one or more genetic variations which result in reduced expression of one or more ATIs, without the presence of a “transgene”. The genetic variation may be artificially introduced using any one or more of the well known techniques described herein, such as random mutagenesis (e.g., TILLING) or site specific genome editing (e.g., CRISPR/Cas).

As used herein, unless stated to the contrary, the phrase “about” refers to any reasonable range in light of the value in question. In a preferred embodiment, the term “about” refers to +1-10%, more preferably +/−5%, more preferably +/−1%, the specified value.

Unless specified otherwise, when referring to weight and a percentage, the units are w/w.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Alpha-Amylase/Trypsin Inhibitors (ATIs)

The phrase “alpha-amylase/trypsin inhibitor”, or “ATI”, as used herein, refers to a class of polypeptides present in cereals, such as barley, which are capable of inhibiting the enzymatic activity of alpha amylase or trypsin. ATIs generally have conserved secondary structure characterized by 4 or 5 intra-chain disulfide bonds (S—S) and alpha-helical structural components, which defines their compact three-dimensional structure. Due to their structural conformation, these proteins typically show high resistance to human digestion mediated by the gastrointestinal proteases trypsin and pepsin.

Cereal seeds contain a number of alpha-amylase inhibitors (AAI). These inhibitors can be grouped into families based on structural similarities. The conserved ‘Plant lipid transfer protein/seed storage protein/trypsin-alpha amylase inhibitor domain family domain’ identifies sequences belonging to the cereal (monocotyledon) ATI protein family.

The cereal ATI protein family consists of proteins of ˜120 amino acids which contain 10 cysteine residues, all of which are typically involved in disulphide bonds. The schematic representation of the structure of these proteins is shown below:

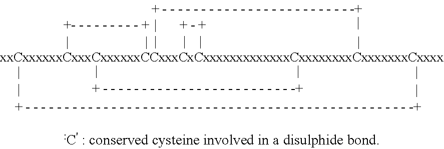

Exemplary ATIs include polypeptides comprising the amino acid sequences provided in SEQ NOs: 86 to 97. These exemplary ATIs include CM proteins, which are a group of at least five salt-soluble components (CMa to CMe) that can be selectively extracted from barley endosperm with chloroform/methanol mixtures. CMa to CMe proteins are encoded by a disperse multigene family. CMa, CMb and CMd inhibitors of alpha-amylase, whilst CMc and CMe are reported to inhibit trypsin.

There is no overall similarity of ATIs to hordeins. Hordeins may be sulphur-rich or sulphur-poor. There are big differences in Cys content even within the hordein super-family. So there is no expectation of altered ATI levels due to altered hordein levels. However, the present inventors have surprisingly found that grain from barley mutants with a reduced hordein content also comprise reduced levels of ATIs, relative to wild-type.

As used herein, the phrase “reduced level of one or more ATIs relative to grain from a corresponding wild-type barley plant” refers to a quantity of at least one ATI protein in the barley grain which is less than the quantity of the at least one ATI protein in the wild-type grain. The level of the one or more ATIs in the barley grain may be reduced by any amount, relative to the wild-type grain. For example, the level of the one or more ATIs in the barley grain can be about 90% or less, about 80% or less, about 70% or less, about 60% or less, about 50% or less, about 40% or less, about 30% or less, about 20% or less, about 10% or less, or about 5% or less of the level in the grain from the corresponding wild-type plant. For the avoidance of doubt, the reduced level also includes grain which completely lacks the one or more ATIs or which comprises undetectable levels of the one or more ATIs.

Example 5 herein describes an independent data acquisition (IDA) LC-MS approach used by the inventors to identify and characterise ATIs present in barley grain. Furthermore, Examples 7 and 8 describe multiple reaction monitoring (MRM/SRM) techniques that were used to perform relative quantitation of the ATIs across seven barley mutant lines compared to wild-type barley. Interestingly the BCD hordein null mutant (also referred to herein as “ULG3.0” or “triple null”) showed unexpected synergistic reductions in the CMa-e proteins compared to the single and double null mutant lines.

### Hordeins

Cereal prolamins (known as gliadins in wheat, hordeins in barley, secalins in rye, avenins in oats, and zeins in maize) are the main endosperm storage proteins in all cereal grains, with the exception of oats and rice (Shewry and Halford, 2002). Hordeins represent 35-50% of the total protein in barley seeds (Jaradat, 1991). They are classified into four groups, A (also known as γ hordein), B, C, and D, in order of decreasing mobility (Field et al., 1982). B, C, D and γ-hordeins are encoded by the Hor2, Hor1, Hor3, and Hor5 loci, respectively, on chromosome 1H. In addition, the lys3 gene in barley encodes a protein called “Barley Prolamin-box Binding Factor” (BPBF) which is associated with C hordein expression. The B hordeins are the main protein fraction, differing from C hordeins in their sulphur content (Kreis and Shewry, 1989). B hordeins account for 70-80% of the total and C hordeins for 10-20% (Davies et al., 1993). The γ hordeins are not generally considered to be a storage fraction, whereas D hordeins are homologous to the high-molecular-weight glutenins. Hordeins, along with the rest of the related cereal prolamins, are not expressed in the zygotic embryo itself, unlike other storage proteins such as napins; they are believed to be expressed exclusively in the starchy endosperm during the middle-to-late stages of seed development.

Examples of barley hordein amino acid sequences and genes encoding them are provided in WO 2009/021285. Examples of barley plants having genetic variations which result in reduced levels of hordeins are provided in WO 2014/197943 and WO 2009/021285.

As used herein, the term “hordeins”, for example when used in the phrase “about 50 ppm or less hordeins” and similar phrases refers to total hordeins including B-, C-, D- and γ-hordeins.

### Malting

A malt-based beverage provided by the present invention involves alcohol beverages (including distilled beverages) and non-alcohol beverages that are produced by using malt as a part or whole of their starting material. Examples include beer, happoshu (low-malt beer beverage), whiskey, low-alcohol malt-based beverages (e.g., malt-based beverages containing less than 1% of alcohols), and non-alcohol beverages.

Malting is a process of controlled steeping and germination followed by drying of the barley grain. This sequence of events is important for the synthesis of numerous enzymes that cause grain modification, a process that principally depolymerizes the dead endosperm cell walls and mobilizes the grain nutrients. In the subsequent drying process, flavour and colour are produced due to chemical browning reactions. Although the primary use of malt is for beverage production, it can also be utilized in other industrial processes, for example as an enzyme source in the baking industry, or as a flavouring and colouring agent in the food industry, for example as malt or as a malt flour, or indirectly as a malt syrup, etc.

In one embodiment, the present invention relates to methods of producing a malt composition. The method preferably comprises the steps of:

(i) providing grain of a barley plant of the invention,

(ii) steeping said grain,

(iii) germinating the steeped grains under predetermined conditions and

(iv) drying said germinated grains.

For example, the malt may be produced by any of the methods described in Hoseney (Principles of Cereal Science and Technology, Second Edition, 1994: American Association of Cereal Chemists, St. Paul, Minn.). However, any other suitable method for producing malt may also be used with the present invention, such as methods for production of speciality malts, including, but not limited to, methods of roasting the malt.

Malt may be prepared using only grain produced from barley plants of the invention or in mixtures comprising other grains.

Malt is mainly used for brewing beer, but also for the production of distilled spirits. Brewing comprises wort production, main and secondary fermentations and post-treatment. First the malt is milled, stirred into water and heated. During this “mashing”, the enzymes activated in the malting degrade the starch of the kernel into fermentable sugars. In the production of beer, the produced wort is clarified, yeast is added, the mixture is fermented and a post-treatment is performed.

In another embodiment, wort compositions can be prepared from the malt. Said wort may be first and/or second and/or further wort. In general a wort composition will have a high content of amino nitrogen and fermentable carbohydrates, mainly maltose. Typically, wort is prepared by incubating malt with water, i.e. by mashing. During mashing, the malt/water composition may be supplemented with additional carbohydrate-rich compositions, for example barley, maize or rice adjuncts. Unmalted cereal adjuncts usually contain no active enzymes, and therefore rely on malt or exogenous enzymes to provide enzymes necessary for sugar conversion.

In general, the first step in the wort production process is the milling of malt in order that water may gain access to grain particles in the mashing phase, which is fundamentally an extension of the malting process with enzymatic depolymerization of substrates. During mashing, milled malt is incubated with a liquid fraction such as water. The temperature is either kept constant (isothermal mashing) or gradually increased. In either case, soluble substances produced in malting and mashing are extracted into said liquid fraction before it is separated by filtration into wort and residual solid particles denoted spent grains. This wort may also be denoted first wort. After filtration, a second wort is obtained. Further worts may be prepared by repeating the procedure. Non-limiting examples of suitable procedures for preparation of wort is described in Hoseney (supra).

The wort composition may also be prepared by incubating barley plants of the invention or parts thereof with one or more suitable enzyme, such as enzyme compositions or enzyme mixture compositions, for example Ultraflo or Cereflo (Novozymes). The wort composition may also be prepared using a mixture of malt and unmalted barley plants or parts thereof, optionally adding one or more suitable enzymes during said preparation.

### Grain Processing

Barley grain of the invention can be processed to produce a food ingredient, beverage ingredient, food, beverage, or non-food product using any technique known in the art.

In one embodiment, the food or food ingredient is processed barley grain. As used herein, “processed barley grain” refers to grain that has been in anyway modified by man. In an embodiment, the grain has been processed such that it is no longer able to germinate. In one embodiment, processed barley grain of the invention has been dehulled. In another embodiment, it has been subjected to heat treatment, such as boiling, so that it is no longer able to germinate.

As will be appreciated by those skilled in the art, “dehulled” barley grain (sometimes also referred to as “hulled” barley), is whole grain barley that has been processed to remove the tough, and mostly inedible, fibrous outer hull of the grain. Dehulled barley can be used as a food ingredient or as a food itself.

In another embodiment, the processed barley grain is pearl barley. As is known in the art, “pearl barley” (also referred to as “pearled barley”) is barley grain that has been processed to remove its fibrous outer hull and has also been polished to remove some or all of the bran layer. Pearl barley can be used as a food ingredient or as a food itself.

In one embodiment, the product is whole grain flour (an ultrafine-milled whole grain flour, such as an ultrafine-milled whole grain flour; a whole grain flour, or a flour made from about 100% of the grain). The whole grain flour includes a refined flour constituent (refined flour or refined flour) and a coarse fraction (an ultrafine-milled coarse fraction).

Refined flour may be flour which is prepared, for example, by grinding and bolting cleaned barley. The Food and Drug Administration (FDA) requires flour to meet certain particle size standards in order to be included in the category of refined barley flour. The particle size of refined flour is described as flour in which not less than 98% passes through a cloth having openings not larger than those of woven wire cloth designated “212 micrometers (U.S. Wire 70)”.

The coarse fraction includes at least one of: bran and germ. For instance, the germ is an embryonic plant found within the barley kernel. The germ includes lipids, fiber, vitamins, protein, minerals and phytonutrients, such as flavonoids. The bran includes several cell layers and has a significant amount of lipids, fiber, vitamins, protein, minerals and phytonutrients, such as flavonoids. Further, the coarse fraction may include an aleurone layer which also includes lipids, fiber, vitamins, protein, minerals and phytonutrients, such as flavonoids. The aleurone layer, while technically considered part of the endosperm, exhibits many of the same characteristics as the bran and therefore is typically removed with the bran and germ during the milling process. The aleurone layer contains proteins, vitamins and phytonutrients, such as ferulic acid.

Further, the coarse fraction may be blended with the refined flour constituent. Preferably, the coarse fraction is homogenously blended with the refined flour constituent. Homogenously blending the coarse fraction and refined flour constituent may help reduce stratification of the particles by size during shipping. The coarse fraction may be mixed with the refined flour constituent to form the whole grain flour, thus providing a whole grain flour with increased nutritional value, fiber content, and antioxidant capacity as compared to refined flour. For example, the coarse fraction or whole grain flour may be used in various amounts to replace refined or whole grain flour in baked goods, snack products, and food products. The whole grain flour of the present invention (i.e.-ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In further embodiments, enzymes found within the bran and germ of the whole grain flour and/or coarse fraction are inactivated in order to stabilize the whole grain flour and/or coarse fraction. It is contemplated by the present invention that inactivated may also mean inhibited, denatured, or the like. Stabilization is a process that uses steam, heat, radiation, or other treatments to inactivate the enzymes found in the bran and germ layer. Naturally occurring enzymes in the bran and germ will catalyze changes to compounds in the flour, adversely affecting the cooking characteristics of the flour and the shelf life. Inactivated enzymes do not catalyze changes to compounds found in the flour, therefore, flour that has been stabilized retains its cooking characteristics and has a longer shelf life. For example, the present invention may implement a two-stream milling technique to grind the coarse fraction. Once the coarse fraction is separated and stabilized, the coarse fraction is then ground through a grinder, preferably a gap mill, to form a coarse fraction having a particle size distribution less than or equal to about 500 micrometers. In an exemplary embodiment, the gap mill tip speed normally operates between 115 m/s to 144 m/s, the high tip speed generates heat. The heat generated during the process and the airflow lead to a decrease in the microbial load of the coarse fraction. In further embodiments, prior to grinding in a gap mill, the coarse fraction may have an average aerobic plate count of 95,000 colony forming units/gram (cfu/g) and an average coliform count of 1,200 cfu/g. After passing through the gap mill the coarse fraction may have an average aerobic plate count of 10,000 cfu/g and an average coliform count of 900 cfu/g. Thus, the microbial load may be noticeably decreased in the coarse fraction of the present invention. After sifting, any ground coarse fraction having a particle size greater than 500 micrometers may be returned to the process for further milling.

In additional embodiments, the whole grain flour or the coarse fraction may be a component of a food product. For example, the food product may be a bagel, a biscuit, a bread, a bun, a croissant, a dumpling, an English muffin, a muffin, a pita bread, a quickbread, a refrigerated/frozen dough products, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In alternative embodiments, the whole grain flour or coarse fraction may be a component of a nutritional supplement. For instance, the nutritional supplement may be a product that is added to the diet containing one or more ingredients, typically including: vitamins, minerals, herbs, amino acids, enzymes, antioxidants, herbs, spices, probiotics, extracts, prebiotics and fiber. The whole grain flour or coarse fraction of the present invention includes vitamins, minerals, amino acids, enzymes, and fiber. For instance, the coarse fraction contains a concentrated amount of dietary fiber as well as other essential nutrients, such as B-vitamins, selenium, chromium, manganese, magnesium, and antioxidants, which are essential for a healthy diet. For example 22 grams of the coarse fraction of the present invention delivers 33% of an individual's daily recommend consumption of fiber. Further, 14 grams is all that is needed to deliver 20% of an individuals daily recommend consumption of fiber. Thus, the coarse fraction is an excellent supplemental source for consumption of an individual's fiber requirement. Therefore, in a present embodiment, the whole grain flour or coarse fraction may be a component of a nutritional supplement. The nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients.

In additional embodiments, the whole grain flour or coarse fraction may be a fiber supplement or a component thereof. Many current fiber supplements such as psyllium husks, cellulose derivatives, and hydrolyzed guar gum have limited nutritional value beyond their fiber content. Additionally, many fiber supplements have an undesirable texture and poor taste. Fiber supplements made from the whole grain flour or coarse fraction may help deliver fiber as well as protein, and antioxidants. The fiber supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage, this embodiment may be particularly attractive as a fiber supplement for children.

In an additional embodiment, a milling process may be used to make a multi-grain flour, multi-barley flour, or a multi-grain coarse fraction. For example, bran and germ from one type of barley may be ground and blended with ground endosperm or whole grain barley flour of another type of barley. Alternatively bran and germ of one type of grain may be ground and blended with ground endosperm or whole grain flour of another type of grain. In an additional embodiment, bran and germ from a first type of barley or grain may be blended with bran and germ from a second type of barley or grain to produce a multi-grain coarse fraction. It is contemplated that the present invention encompasses mixing any combination of one or more of bran, germ, endosperm, and whole grain flour of one or more grains. This multi-grain, multi-barley approach may be used to make custom flour and capitalize on the qualities and nutritional contents of multiple types of grains or barleys to make one flour.

The whole grain flour of the present invention may be produced via a variety of milling processes. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. In one embodiment, the grinder may be a gap mill. After grinding, the grain is discharged and conveyed to a sifter. Any sifter known in the art for sifting a ground particle may be used. Material passing through the screen of the sifter is the whole grain flour of the present invention and requires no further processing. Material that remains on the screen is referred to as a second fraction. The second fraction requires additional particle reduction. Thus, this second fraction may be conveyed to a second passage grinder. After grinding, the second fraction may be conveyed to a second sifter. Material passing through the screen of the second sifter is the whole grain flour of the present invention. The material that remains on the screen is referred to as the fourth fraction and requires further processing to reduce the particle size. The fourth fraction on the screen of the second sifter is conveyed back into either the first passage grinder or the second passage grinder for further processing via a feedback loop. In an alternative embodiment of the invention, the process may include a plurality of first pass grinders to provide a higher system capacity.

It is contemplated that the whole grain flour, coarse fraction and/or grain products of the present invention may be produced by any milling process known in the art. Further, it is contemplated that the whole grain flour, coarse fraction and/or grain products of the present invention may be modified or enhanced by way of numerous other processes such as: fermentation, instantizing, extrusion, encapsulation, toasting, roasting, or the like.

Generation of Plants with Genetic Variations

There are many techniques known in the art which can be used to produce barley with reduced levels of ATIs by introducing one more more genetic variations. These techniques include recombinant DNA technology to produce transgenic plants, random mutagenesis, and site specific gene editing techniques.

In an embodiment, the plants are homozygous for each and every genetic variation i.e., their progeny do not segregate for the desired phenotype. The plants may also be heterozygous for the genetic variation(s), such as, for example, in F1 progeny which have been grown from hybrid seed. Such plants may provide advantages such as hybrid vigour, well known in the art.

It is also to be understood that two different plants comprising genetic variations can also be mated to produce offspring that contain two independently segregating genetic variations. Selfing of appropriate progeny can produce plants that are homozygous for each genetic variation. Back-crossing to a parental plant and out-crossing with a wild-type plant is also contemplated, as is vegetative propagation. Descriptions of other breeding methods that are commonly used for different traits and crops can be found in Fehr (1987).

The development or regeneration of plants containing an introduced genetic variation is well known in the art. Preferably, the regenerated plants are self-pollinated to provide homozygous plants for the genetic variation(s). Otherwise, pollen obtained from the regenerated plants is crossed to seed-grown plants of agronomically important lines. Conversely, pollen from plants of these important lines is used to pollinate regenerated plants. A plant of the present invention containing a desired genetic variation(s) is cultivated using methods well known to one skilled in the art.

To confirm the presence of a genetic variation, a polymerase chain reaction (PCR) amplification or Southern blot analysis can be performed using methods known to those skilled in the art. Expression products can be detected in any of a variety of ways, depending upon the nature of the product, and include Western blot and enzyme assay. One particularly useful way to quantitate protein expression and to detect replication in different plant tissues is to use a reporter gene, such as GUS. Once plants comprising the desired genetic variation have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

Suitable endogenous gene targets for mutagenesis/modification to reduce levels of ATIs in barley grain include the lys3 gene which encodes Barley Prolamin-box Binding Factor (BPBF, described in Moehs et al., 2019; and Orman-Ligeza et al., 2019), optionally in combination with Hor2 and Hor3. Other suitable targets include the genes encoding the ATI protein sequences provided in SEQ ID NOs: 86 to 97.

### Tilling

Plants of the invention can be produced using the process known as TILLING (Targeting Induced Local Lesions IN Genomes). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005) and Henikoff et al., (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Genome Editing Using Site-Specific Nucleases

Genome editing uses engineered nucleases composed of sequence specific DNA binding domains fused to a non-specific DNA cleavage module. These chimeric nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption.

Engineered nucleases useful in the methods of the present invention include zinc finger nucleases (ZFNs) and transcription activator-like (TAL) effector nucleases (TALEN).

Typically nuclease encoded genes are delivered into cells by plasmid DNA, viral vectors or in vitro transcribed mRNA. The use of fluorescent surrogate reporter vectors also allows for enrichment of ZFN- and TALEN-modified cells. As an alternative to ZFN gene-delivery systems, cells can be contacted with purified ZFN proteins which are capable of crossing cell membranes and inducing endogenous gene disruption.

Complex genomes often contain multiple copies of sequences that are identical or highly homologous to the intended DNA target, potentially leading to off-target activity and cellular toxicity. To address this, structure (Miller et al., 2007; Szczepek et al., 2007) and selection based (Doyon et al., 2011; Guo et al., 2010) approaches can be used to generate improved ZFN and TALEN heterodimers with optimized cleavage specificity and reduced toxicity.

A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell or organism. Typically, a ZFN having more than three zinc fingers would have progressively greater specificity with each additional zinc finger.

The zinc finger domain can be derived from any class or type of zinc finger. In a particular embodiment, the zinc finger domain comprises the Cis₂His₂ type of zinc finger that is very generally represented, for example, by the zinc finger transcription factors TFIIIA or Sp1. In a preferred embodiment, the zinc finger domain comprises three Cis₂His₂ type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques. (see, for example, Bibikova et al., 2002).

The ZFN DNA-cleavage domain is derived from a class of non-specific DNA cleavage domains, for example the DNA-cleavage domain of a Type II restriction enzyme such as Fold (Kim et al., 1996). Other useful endonucleases may include, for example, HhaI, HindIII, Nod, BbvCI, EcoRI, BglI, and AlwI.

A linker, if present, between the cleavage and recognition domains of the ZFN comprises a sequence of amino acid residues selected so that the resulting linker is flexible. Or, for maximum target site specificity, linkerless constructs are made. A linkerless construct has a strong preference for binding to and then cleaving between recognition sites that are 6 bp apart. However, with linker lengths of between 0 and 18 amino acids in length, ZFN-mediated cleavage occurs between recognition sites that are between 5 and 35 bp apart. For a given linker length, there will be a limit to the distance between recognition sites that is consistent with both binding and dimerization. (Bibikova et al., 2001). In a preferred embodiment, there is no linker between the cleavage and recognition domains, and the target locus comprises two nine nucleotide recognition sites in inverted orientation with respect to one another, separated by a six nucleotide spacer.

In order to target genetic recombination or mutation according to a preferred embodiment of the present invention, two 9 bp zinc finger DNA recognition sequences must be identified in the host DNA. These recognition sites will be in an inverted orientation with respect to one another and separated by about 6 bp of DNA. ZFNs are then generated by designing and producing zinc finger combinations that bind DNA specifically at the target locus, and then linking the zinc fingers to a DNA cleavage domain.

ZFN activity can be improved through the use of transient hypothermic culture conditions to increase nuclease expression levels (Doyon et al., 2010) and co-delivery of site-specific nucleases with DNA end-processing enzymes (Certo et al., 2012). The specificity of ZFN-mediated genome editing can be improved by use of zinc finger nickases (ZFNickases) which stimulate HDR without activation the error-prone NHE-J repair pathway (Kim et al., 2012; Wang et al., 2012; Ramirez et al., 2012; McConnell Smith et al., 2009).

A transcription activator-like (TAL) effector nuclease (TALEN) comprises a TAL effector DNA binding domain and an endonuclease domain.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

### Genome Editing Using Programmable RNA-Guided DNA Endonucleases

Distinct from the site-specific nucleases described above, the clustered regulatory interspaced short palindromic repeats (CRISPR)/Cas system provides an alternative to ZFNs and TALENs for inducing targeted genetic alterations. In bacteria, the CRISPR system provides acquired immunity against invading foreign DNA via RNA-guided DNA cleavage.

CRISPR systems rely on CRISPR RNA (crRNA) and transactivating chimeric RNA (tracrRNA) for sequence-specific silencing of invading foreign DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

The CRISPR system can be portable to plant cells by co-delivery of plasmids expressing the Cas endonuclease and the necessary crRNA components. The Cas endonuclease may be converted into a nickase to provide additional control over the mechanism of DNA repair.

CRISPR loci are a distinct class of interspersed short sequence repeats (SSRs) that were first recognized in E. coli (Ishino et al., 1987; Nakata et al., 1989). Similar interspersed SSRs have, been identified in Haloferax mediterranei, Streptococcus pyogenes, Anabaena, and Mycobacterium tuberculosis (Groenen et al., 1993; Hoe et al., 1999; Masepohl et al., 1996; Mojica et al., 1995).

The CRISPR loci differ from other SSRs by the structure of the repeats, which have been termed short regularly spaced repeats (SRSRs) (Janssen et al., 2002; Mojica et al., 2000). The repeats are short elements that occur in clusters, that are always regularly spaced by unique intervening sequences with a constant length (Mojica et al., 2000). Although the repeat sequences are highly conserved between strains, the number of interspersed repeats and the sequences of the spacer regions differ from strain to strain (van Embden et al., 2000).

The common structural characteristics of CRISPR loci are described in Jansen et al., (2002) as (i) the presence of multiple short direct repeats, which show no or very little sequence variation within a given locus; (ii) the presence of non-repetitive spacer sequences between the repeats of similar size; (iii) the presence of a common leader sequence of a few hundred basepairs in most species harbouring multiple CRISPR loci; (iv) the absence of long open reading frames within the locus; and (v) the presence of one or more cas genes.

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

As used herein, the term “cas gene” refers to one or more cas genes that are generally coupled associated or close to or in the vicinity of flanking CRISPR loci. A comprehensive review of the Cas protein family is presented in Haft et al. (2005). The number of cas genes at a given CRISPR locus can vary between species.

### Transgenic Plants

In some embodiments, the barley grain described herein is from a transgenic plant. Transgenic barley plants, as defined in the context of the present invention include plants (as well as parts and cells of said plants) and their progeny which comprise a transgene which causes production of at least one polynucleotide and/or polypeptide in the desired plant or plant part. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

Four general methods for direct delivery of a transgene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. Nos. 4,945,050 and 5,141,131); (3) viral vectors (Clapp, 1993; Lu et al., 1993; Eglitis et al., 1988); and (4) receptor-mediated mechanisms (Curiel et al., 1992; Wagner et al., 1992). Emerging methods for direct delivery of a transgene or nucleases to conduct editing include using carbon nanotubes as described by Demirer et al. (2019).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. An illustrative embodiment of a method for delivering DNA into Zea mays cells by acceleration is a biolistics α-particle delivery system, that can be used to propel particles coated with DNA through a screen, such as a stainless steel or Nytex screen, onto a filter surface covered with corn cells cultured in suspension. A particle delivery system suitable for use with the present invention is the helium acceleration PDS-1000/He gun is available from Bio-Rad Laboratories.

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus that express the exogenous gene product 48 hours post-bombardment often range from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265).

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions that influence the physiological state of the recipient cells and that may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

Modern Agrobacterium transformation vectors are capable of replication in E. coli as well as Agrobacterium, allowing for convenient manipulations as described (Klee et al., 1985). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

Methods for transforming dicots, primarily by use of Agrobacterium tumefaciens, and obtaining transgenic plants have been published for cotton (U.S. Pat. Nos. 5,004,863, 5,159,135, 5,518,908); soybean (U.S. Pat. No. 5,569,834, 5,416,011); Brassica (U.S. Pat. No. 5,463,174); peanut (Cheng et al., 1996); and pea (Grant et al., 1995).

Methods for transformation of cereal plants such as barley for introducing exogenous nucleic acids and for regeneration of plants from protoplasts or immature plant embryos are well known in the art, see for example, CA 2,092,588, AU 61781/94, AU 667939, U.S. Pat. No. 6,100,447, PCT/US97/10621, U.S. Pat. Nos. 5,589,617, 6,541,257, and WO 99/14314. Preferably, transgenic barley plants are produced by Agrobacterium tumefaciens mediated transformation procedures. Vectors carrying the desired nucleic acid construct may be introduced into regenerable barley cells of tissue cultured plants or explants, or suitable plant systems such as protoplasts.

The regenerable barley cells are preferably from the scutellum of immature embryos, mature embryos, callus derived from these, or the meristematic tissue.

Polynucleotides which Down-Regulate the Production of an ATI

In one embodiment, the barley grain described herein comprises a genetic variation (e.g., encoded by a transgene) which down-regulates the production of at least one ATI in the grain. Alternatively or additionally, the barley grain may comprise a genetic variation (e.g., encoded by a transgene) which down-regulates the production of at least one hordein in the grain. Examples of such polynucleotides include, but are not limited to, antisense polynucleotide, a sense polynucleotide, a catalytic polynucleotide, an artificial microRNA or a duplex RNA molecule. When present in the grain, each of these polynucleotides result in a reduction in ATI mRNA available for translation.

Suitable gene targets for downregulation to reduce levels of ATIs in barley grain include the lys3 gene which encodes Barley Prolamin-box Binding Factor (BPBF, described in Moehs et al., 2019; and Orman-Ligeza et al., 2019), optionally in combination with Hor2 and Hor3. Other suitable targets include the genes encoding the ATI protein sequences provided in SEQ ID NOs: 86 to 97.

### Antisense Polynucleotides

The term “antisense polynucletoide” shall be taken to mean a DNA or RNA, or combination thereof, molecule that is complementary to at least a portion of a specific mRNA molecule encoding an ATI and capable of interfering with a post-transcriptional event such as mRNA translation. The use of antisense methods is well known in the art (see for example, G. Hartmann and S. Endres, Manual of Antisense Methodology, Kluwer (1999)). The use of antisense techniques in plants has been reviewed by Bourque (1995) and Senior (1998). Senior (1998) states that antisense methods are now a very well established technique for manipulating gene expression.

An antisense polynucleotide in a barley plant of the invention will hybridize to a target polynucleotide under physiological conditions. As used herein, the term “an antisense polynucleotide which hybridises under physiological conditions” means that the polynucleotide (which is fully or partially single stranded) is at least capable of forming a double stranded polynucleotide with mRNA encoding a protein, such as a barley ATI under normal conditions in a barley cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988, Perriman et al., 1992) and the hairpin ribozyme (Shippy et al., 1999).

The ribozymes in barley plants of the invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, the catalytic polynucleotides should also be capable of hybridizing a target nucleic acid molecule (for example an mRNA encoding a barley ATI) under “physiological conditions”, namely those conditions within a barley cell.

### RNA Interference

RNA interference (RNAi) is particularly useful for specifically inhibiting the production of a particular protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and antisense sequences are flanked by an unrelated sequence which enables the sense and antisense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815. In particular, Kalunke et al. (2020) describe RNAi constructs for silencing wheat ATI genes CM3, CM16 and 0.28. These could be used to design similar RNAi constructs for silencing the homologous ATI genes in barley identified by the present inventors, i.e., uniprot accession numbers P11643 (SEQ ID NO: 91), P32936 (SEQ ID NO: 93) and P13691 (SEQ ID NO: 89) respectively.

In one example, a DNA is introduced that directs the synthesis of an at least partly double stranded (duplex) RNA product(s) with homology to the target gene to be inactivated. The DNA therefore comprises both sense and antisense sequences that, when transcribed into RNA, can hybridize to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double-stranded region may comprise one or two RNA molecules, transcribed from either one DNA region or two. The presence of the double stranded molecule is thought to trigger a response from an endogenous plant system that destroys both the double stranded RNA and also the homologous RNA transcript from the target plant gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

Preferred small interfering RNA (“siRNA”) molecules comprise a nucleotide sequence that is identical to about 19-21 contiguous nucleotides of the target mRNA. Preferably, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the barley plant in which it is to be introduced, e.g., as determined by standard BLAST search.

microRNA

MicroRNA regulation is a clearly specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005).

### Cosuppression

Another molecular biological approach that may be used is co-suppression. The mechanism of co-suppression is not well understood but is thought to involve post-transcriptional gene silencing (PTGS) and in that regard may be very similar to many examples of antisense suppression. It involves introducing an extra copy of a gene or a fragment thereof into a plant in the sense orientation with respect to a promoter for its expression. The size of the sense fragment, its correspondence to target gene regions, and its degree of sequence identity to the target gene are as for the antisense sequences described above. In some instances the additional copy of the gene sequence interferes with the expression of the target plant gene. Reference is made to WO 97/20936 and EP 0465572 for methods of implementing co-suppression approaches.

### Nucleic Acid Constructs

Nucleic acid constructs useful for producing transgenic plants can readily be produced using standard techniques.

When inserting a region encoding an mRNA the construct may comprise intron sequences. These intron sequences may aid expression of the transgene in the plant.

The term “intron” is used in its normal sense as meaning a genetic segment that is transcribed but does not encode protein and which is spliced out of an RNA before translation. Introns may be incorporated in a 5′-UTR or a coding region if the transgene encodes a translated product, or anywhere in the transcribed region if it does not. However, in a preferred embodiment, any polypeptide encoding region is provided as a single open reading frame. As the skilled addressee would be aware, such open reading frames can be obtained by reverse transcribing mRNA encoding the polypeptide.

To ensure appropriate expression of the gene encoding an mRNA of interest, the nucleic acid construct typically comprises one or more regulatory elements such as promoters, enhancers, as well as transcription termination or polyadenylation sequences. Such elements are well known in the art.

The transcriptional initiation region comprising the regulatory element(s) may provide for regulated or constitutive expression in the plant. Preferably, expression at least occurs in cells of the seed.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants; see, e.g., WO 84/02913. All of these promoters have been used to create various types of plant-expressible recombinant DNA vectors.

The promoter may be modulated by factors such as temperature, light or stress. Ordinarily, the regulatory elements will be provided 5′ of the genetic sequence to be expressed. The construct may also contain other elements that enhance transcription such as the nos 3′ or the ocs 3′ polyadenylation regions or transcription terminators.

The 5′ non-translated leader sequence can be derived from the promoter selected to express the heterologous gene sequence, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to the use of constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. Nos. 5,362,865 and 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Typically, the nucleic acid construct comprises a selectable marker. Selectable markers aid in the identification and screening of plants or cells that have been transformed with the exogenous nucleic acid molecule. The selectable marker gene may provide antibiotic or herbicide resistance to the barley cells, or allow the utilization of substrates such as mannose. The selectable marker preferably confers hygromycin resistance to the barley cells.

Preferably, the nucleic acid construct is stably incorporated into the genome of the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes the use of a recombinant vector, which includes at least transgene outlined herein, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

### EXAMPLES

### Example 1. Materials and methods

### Plant Material

Plants of barley (Hordeum vulgare) cultivars Hindmarsh and Commander were obtained from Australian Winter Cereals Collection (Tamworth, Australia). Plants of barley cultivar Sloop was obtained from the Australian Cereals Collection (Tamworth, Australia). The selected barley varieties Risø 56 (expressing no B-hordeins), Risø 1508 (expressing no C-hordeins and decreased D- and B-hordeins) (Doll, 1973; Doll, 1983) were obtained from the Nordic Germplasm Bank (Alnarp, Sweden). Risø 1508 carries the lys3a mutation which is an ethyleneimine induced mutant carrying a mutation in the lys3 gene on chromosome 5H which reduced accumulation of C-hordeins. A line which did not express the D-hordein gene, Ethiopian R118 (Brennan et al., 1998), was obtained from The John Innes Centre Public Collections, Norwich. Each of these lines are publicly available.

The barley lines used here are wild-type barley cv Sloop (field grown is indicated by ‘WTf’ and glass-house grown indicated by ‘WTg’), the individual hordein nulls (B-, C- and D-), the double-nulls (BC-, BD- and CD-), and the triple-null (BCD-) were used for independent data acquisition (IDA) and multiple (selected) reaction monitoring (MRM-MS). The BC-double null mutant barley plants are described in WO 2009/021285 and the the triple-null (BCD-) mutant barley plant is described in WO 2014/197943.

In the glasshouse, plants were grown in 15 cm pots under standard glasshouse conditions with natural daylight and a temperature regime of 25° C. maximum during the day and 15° C. minimum at night. To provide barley leaf tissue for gene expression studies, grain was germinated in the lab in vermiculite and the first leaf was harvested after 7 days. For the grain development protein and gene expression studies, heads of greenhouse grown plants were tagged at anthesis and grain was harvested every 4 days post anthesis (DPA). The whole caryopsis was used at 0 and 4 days post anthesis and the embryo and pericarp were removed from all other samples except the 28 day sample from which the pericarp could not be removed.

For field analyses, plants were grown side by side at the CSIRO Ginninderra Experimental Station (GES), Canberra Australia. Wild-type Sloop, and null lines in Sloop background, containing single, B, C or D deletions, and double null BD, CD and BC deletions and the triple BDC were planted in 1 metre rows. Planting occurred in July (winter) and were harvested in the following December (summer). The plants were hand irrigated twice over the growing season, and hand harvested.

### Milling Grain to Produce Flour

The moisture content of barley grain was measured by NIR using a FOSS 5000 (Foss A/S, Denmark) machine according to the manufacturer's instruction and then conditioned to 14% moisture by mixing with the required amount of water overnight and then milled using a Metefem Hungarian Mill (model FQD2000, Hungary). Fine flour was obtained by sieving the wholemeal with a 300 μm sieve (Endecotts Pty Ltd Sieves, London England).

### ATI Extraction and Digestion

The ATIs and gluten proteins belong to separate protein families but are both found in the grain endosperm and are commonly detected in the same fraction after extraction from the grain (Makharia et al., 2015). In order to extract the maximum level of ATIs from grain, the inventors developed and optimised two-step extraction protocol: IPA/DTT (which was previously shown to selectively extract hordeins from barley) followed by urea-buffer (a commonly used buffer to extract total proteins from cereal grains) (Bose et al., 2019). The barley variety, cv Commander was used to establish the optimised ATI extraction protocol. First, 20 mg of the milled barley flour (n=4) were weighed and 200 μL of 55% IPA/2% DTT was added. The sample tubes were vortexed until the flour was thoroughly mixed with the solution. The samples were then sonicated for 5 min prior at room temperature and incubated at 50° C. for 30 min. The sample tubes were centrifuged for 10 minutes at 20,800×g, and 200 μL supernatant was transferred to a fresh tube. Subsequently, 200 μL of urea buffer was added to the pellet and mixed by continuous vortexing followed by sonication for 5 min. Samples were kept on a mixer (400 rpm) at room temperature for 45 minutes and then centrifuged for 15 min at 20,800×g. The supernatant was removed and pooled with the IPA/DTT extracted protein sample tube, and vortex mixed. 200 μL of supernatant was transferred to a 10 kDa MWCO filter unit for protein digestion as described in Colgrave et al. (2015). Briefly, the protein on the filter was washed twice with a buffer consisting of 8 M urea in 0.1M Tris-HCL (pH 8.5). Iodoacetamide (50 mM, 100 μL) prepared in 8 M urea and 100 mM Tris-HCl was added to the filters for cysteine alkylation with incubation in the dark for 20 min before centrifugation for 10 min at 20,800×g. The buffer was exchanged with 100 mM ammonium bicarbonate (pH 8.0) by two consecutive wash/centrifugation steps. The digestion enzyme, bovine trypsin (Sigma-Aldrich, NSW, Australia), was prepared as 250 μg/mL in 100 mM ammonium bicarbonate and 200 μL (20:1 w/w protein to enzyme ratio) was added to each filter with overnight incubation at 37° C. The filters were transferred to fresh collection tubes and centrifuged for 15 min at 20,800×g. The filters were washed with 200 μL of 100 mM ammonium bicarbonate, and the combined filtrates were lyophilized (Bose et al., 2019). Digested peptides were resuspended in 100 μL of 1% formic acid before analysis by LC—MS/MS as previously described (Colgrave et al., 2016).

### Protein Analyses

### Global Proteomic Profiling and Protein Identification

Digested samples were pooled (5 μL from each replicate, n=4) prior to analysis, with chromatographic separation using a micro HPLC system (Shimadzu Scientific, Rydalmere, Australia) directly coupled to a 6600 TripleTOF MS (SCIEX, Redwood City, USA) as described previously in Colgrave at al. (2017).

### Analysis of Mass Spectra and Database Searching

ProteinPilot™ 5.0 software (SCIEX) with the Paragon Algorithm (Shilov et al., 2007) was used for protein identification. Tandem mass spectrometry data was searched against in silico proteolytic digests of Poaceae proteins of the Uniprot database (version 2017/02; 2,891,190 sequences) appended with the common repository of adventitious proteins (cRAP) database. The search parameters were defined as iodoacetamide modified for cysteine alkylation and trypsin as the digestion enzyme. ProteinPilot generates a score for each protein based on the confidence, wherein a confidence of 99% is assigned a score of 2.00 and a confidence of 95% is assigned a score of 1.30. The database search results from the combined barley cultivar analyses were manually curated to yield the protein identifications using a 1% global false discovery rate (FDR) determined by the in-built FDR tool within ProteinPilot software (Tang et al., 2008).

### Protein Alignment and Identification of ATI Peptides

Peptide summaries generated by ProteinPilot were used to select peptides that yielded intense peaks and were fully tryptic, i.e., no variable or missed cleavages. MRM transitions were determined for each peptide where the precursor ion (Q1) m/z and the fragment ion (Q3) m/z values were determined from the data collected in the discovery experiments. In total, 54 peptides were selected for MRM experiments.

Initially, four MRM transitions per peptide were analysed in MRM experiments. From these, three MRM transitions were selected per peptide for use in the final method based on transition intensity and lack of interferences (Bose et al., 2019). The multiple reaction monitoring (MRM) technique was used to determine relative quantitation of the ATIs across the seven ULG lines compared to wild-type barley grown under two different environmental conditions.

### MRM/Targeted MS Method Development

The MRM technique was used to perform relative quantitation of the ATIs across ULG lines. Reduced and alkylated digested peptides (20 μL) were chromatographically separated on a Shimadzu Nexera UHPLC and analysed on a 6500 QTRAP mass spectrometer (SCIEX) as described by Colgrave et al. (2016) with minor modifications. Relative quantitation was achieved using scheduled MRM scanning experiments using a 60 s detection window for each MRM transition with RT as determined in the preliminary MRM experiment and a 0.3 s cycle time. Peak area integration of each peptide was conducted using Skyline software v. 19.1 (MacLean et al., 2010). Three commonly observed sample preparation modifications, i.e. carbamidomethyl (C), oxidation (M) and Gln->pyro-Glu (N-term Q), were allowed for each peptide and all transitions were required to co-elute at the same retention time (RT, min). The peak areas of the three MRM transitions monitored were summed and the data was converted to a percentage relative to the average peak area (of all extracts and fractions) for ease of data comparison. Relative expression of the peptides calculated from average peak area was graphed using GraphPad Prism v6.

Prolamin Extraction from Flour

To extract prolamins (alcohol-soluble proteins), grain was milled to wholemeal flour using standard techniques. Prolamins in aqueous washed wholemeal flour (10 g) were dissolved in 55% (v/v) propan-2-ol (HPLC grade), 2% (w/v) dithiothreitol (DTT) by incubation at 65° C. for 45 min, and precipitated with two volumes of propan-2-ol at −20° C. overnight. The precipitated prolamins were dissolved in 8 M urea, 1% DTT, 25 mM triethanolamine-HCl (pH 6), and purified by fast protein liquid chromatography (FPLC) on a 4 mL Resource RPC column (GE Healthcare, Sydney, NSW, Australia) eluted with a 30 mL linear gradient (at 2 mL/min) from 3% to 60% acetonitrile in 1% (v/v) trifluoroacetic acid (TFA).

### Filter-Aided Sample Preparation (FASP) Method

Fifty μL of hordein extract was transferred to a PALL Nanosep 10 MWCO filter. 200 μL of 8 M urea (Sigma) in 0.1 M Tris/HCl pH 8.5 (UA) was added and the mixture centrifuged at 14000 rpm for 15 min at about 20° C. The flow-through from the tube was discarded. A further 200 μl of UA was added to the filter unit and re-centrifuged at 14,000 rpm for 15 min. 100 μl IAA solution (0.05 M iodoacetamide in UA) was added and the sample mixed at 600 rpm in a thermo-mixer set at 20° C. for 1 min and then incubated without mixing for 20 min. 100 μl of UA was added to the filter unit and centrifuged at 14,000×g for 15 min. This step was repeated twice. 100 μl of 0.05M NH₄HCO₃ (ammonium bicarbonate, ABC) in water was added to the filter unit and centrifuged at 14,000×g for 10 min. This step was repeated twice. 40 μl ABC with 15 μL of trypsin stock (1.5 μg/μl, Sigma) was added and each sample mixed at 600 rpm in a thermo-mixer for 1 min. The filter units were incubated in a wet chamber at 37° C. for 4-18 h for enzyme digestion. The filter units were transferred to new collection tubes and centrifuged at 14,000 rpm, for 10 min. 40 μl ABC was added and the filter units centrifuged again at 14,000 rpm for 10 min. This step was repeated once. The flow through fraction was acidified by adding 15 μL of 5% formic acid and lyophilised. The extracted, dried peptides were resuspended in 30 μL 0.5% formic acid for analysis by MRM.

### Preparation of Wort and Beer

Barley was malted in a Joe White Micromalting System in several 800 g tins. The steeping regime involved: 8 h soaking, 9 h rest, 5 h soaking at 17° C. (Sloop); 8 h soaking, 10 h rest, 5 h soaking at 17° C. (Risø 56); and 7 h soaking, 8 h rest, 3 h soaking at 17° C. (Risø 1508). Germination occurred over 94 h at 16° C. for Sloop and 15° C. for the two hordein deletion mutants. The kiln program was over 21 h between 50-80° C. The kilned malt was mashed as detailed in Colgrave et al. (2012). After the indicated amylase rest time, the mash was brought to the boil and boiled for 1 h to produce the wort. During boiling, the boiling wort was bittered with Tettnang hops to achieve 21-22 IBUs. The wort was cooled overnight to 20° C. and then fermented with Fermentis US-05 yeast at 18-20° C. to completion after about 2 weeks. The unfiltered beer was kegged, and force carbonated before bottling.

### Analysis of Beer

A selection of 60 commercial beers were obtained as listed in Supplementary Table 1 of Colgrave et al. (2012). Triplicate samples (1 mL) were taken from two different bottles of each and degassed to remove CO₂ under reduced pressure. Aliquots (100 μL) of degassed beer were taken and were reduced by addition of 20 μL of 50 mM DTT under N₂ for 30 min at 60° C. To these solutions, 20 μL of 100 mM iodoacetamide (IAM) was added and the samples were incubated for 15 min at room temperature. To each solution 5 μL of 1 mg/mL trypsin (Sigma) or chymotrypsin (Sigma) was added and the samples incubated at 37° C. overnight. The digested peptide solution was acidified by addition of 10 μl of 5% formic acid and passed through a 10 kDa MW filter (Pall, Australia). The filtrate was lyophilized and reconstituted in 1% formic acid and stored at 4° C. until analysis.

### Analysis of Undigested Wort and Beer

Wort and beer (0.1 mL) derived from the wild-type (Sloop) barley and hordein deletion mutants were passed through a 10 kDa molecular weight cut-off filter (Pall) by centrifugation at 14,000 rpm for 30 min to produce a peptide fraction amenable to LC-MS/MS. The peptide fraction (10 μL) was analysed on the QStar Elite mass spectrometer.

### Q-TOF MS

Samples were chromatographically separated on a Shimadzu nano HPLC system (Shimadzu Scientific, Rydalmere, Australia) using a Vydac MS C18 300 A, column (150 mm×0.3 mm) with a particle size of 5 μm (Grace Davison, Deerfield, USA) using a linear gradient of 2-42% solvent B over 20 min at a flow rate of 3 μL/min. The mobile phases consisted of solvent A (0.1% formic acid) and solvent B (0.1% formic acid/90% acetonitrile/10% water). A QStar Elite QqTOF mass spectrometer (Applied Biosystems) was used in standard MS/MS data-dependent acquisition mode with a nano-electrospray ionization source. Survey MS spectra were collected (m/z 400-1800) for 1 s followed by three MS/MS measurements on the most intense parent ions (10 counts/second threshold, 2+ to 5+ charge state, and m/z 100-1600 mass range for MS/MS), using the manufacturer's ‘Smart Exit’. Parent ions previously targeted were excluded from repetitive MS/MS acquisition for 30 seconds (mass tolerance of 100 mDa).

### Linear Ion Trap (Triple Quadrupole) MS

Reduced and alkylated tryptic peptides were analyzed on an Applied Biosystems 4000 QTRAP mass spectrometer (Applied Biosystems, Framingham, Mass., USA) equipped with a TurboV ionization source operated in positive ion mode. Samples were chromatographically separated on a Shimadzu Nexera UHPLC (Shimadzu) using a Phenomenex Kinetex C18 (2.1 mm×10 cm) column with a linear gradient of 5-45% acetonitrile (ACN) over 15 min with a flow rate of 400 μL/min. The eluent from the HPLC was directly coupled to the mass spectrometer. Data were acquired and processed using Analyst 1.5 Software™. Information Dependent Acquisition (IDA) analyses were performed using an enhanced MS (EMS) scan over the mass range 350-1500 as the survey scan and triggered the acquisition of tandem mass spectra. The top two ions of charge state 2-5 that exceeded a defined threshold value (100,000 counts) were selected and first subjected to an enhanced resolution (ER) scan prior to acquiring an enhanced product scan (EPI) over the mass range 125-1600.

### Analysis of Mass Spectra and Database Searching

ProteinPilot™ 4.0 software (Applied Biosystems) with the Paragon Algorithm was used for the identification of proteins. Tandem mass spectrometry data was searched against in silico tryptic or chymotryptic digests of Triticeae proteins of the Uniprot (version 2011/05) and NCBI (version 2011/05) databases. All search parameters were defined as iodoacetamide modified with cysteine alkylation, with either trypsin or chymotrypsin as the digestion enzyme. Modifications were set to the “generic workup” and “biological” modification sets provided with this software package, which consisted of 126 possible modifications, for example, acetylation, methylation and phosphorylation. The generic workup modifications set contains 51 potential modifications that may occur as a result of sample handling, for example, oxidation, dehydration and deamidation. Peptides with one missed cleavage were included in the analysis.

### Construction and Application of a Custom-Built Cereal Database

A non-redundant custom cereal seed storage protein database was constructed by including all reported protein sequences from nucleotide entries in NCBI, TIGR Gene Indices, or TIGR Plant Transcript Assemblies belonging to Triticum, Hordeum, Avena, Secale and Triticosecale species. The nucleotide sequences, for the above species, were translated in six frames, trimmed to keep only the longest open reading frame. The resulting protein sequence set was then made non-redundant. Only sequences with 100% match from start to finish were collapsed together, to maintain all variations. Lastly, these files were filtered to retain only entries containing the words gluten, gliadin, glutenin, hordein, avenin or secalin. Tandem mass spectrometry data was searched against the custom cereal database.

### Protein Alignment and Identification of Prototypic Peptides

All known hordein proteins in the Uniprot database and predicted hordein proteins in the TIGR database were aligned. Within each family (B, C, D or y), peptides that were common were selected as representative of the family. MRM transitions were determined for each peptide where the precursor ion (Q1) m/z was based on the size and expected charge and the fragment ion (Q3) m/z values were predicted using known fragmentation patterns and/or the data collected in the characterization workflows. Up to six transitions were used in the preliminary analyses and the MRM transitions were refined and the top two MRM transitions were selected per peptide for use in the final method, wherein the most intense MRM transition was used as a quantifier and the second most intense transition was used as a qualifier.

### MRM Mass Spectrometry

MRM experiments were used for quantification of the hordein-derived tryptic peptides. For both IDA- and MRM-triggered MS/MS experiments, the scan speed was set to 1000 Da/s and peptides were fragmented in the collision cell with nitrogen gas using rolling collision energy dependent on the size and charge of the precursor ion. Quantification of hordein peptides was achieved using scheduled MRM scanning experiments using a 120 s detection window for each MRM transition and a 1 s cycle time. The first quadrupole was used to select the mass-to-charge ratio (m/z) of the analyte, the so-called precursor ion. The precursor ion was then transmitted to the collision cell (the second quadrupole). Collision-induced dissociation (CID) occurs resulting in the production of fragment ions that were transmitted to the third quadrupole. A second stage of mass selection occurs specifically targeting the m/z values of the known fragment ions. The two stages of mass selection are known as Q1 and Q3 referring to the quadrupole in which they occur. The Q1 to Q3 transition is thus known as the MRM transition and is highly specific and selective for the analyte of choice.

### Relative Quantification of Hordeins

The relative quantification of each hordein was performed by integrating the peak area of the most intense MRM transition for each peptide. The average peak area was determined by taking the mean of two replicate injections (on different days) from bottles A and B (representing the biological replicates). The results are presented as the percentage of each hordein protein relative to the average hordein content of all gluten-containing beers.

### Example 2. Generation of a Barley Mutant Having Reduced Levels of ATIs

F5 plants of the Hor2-lys3a double mutant barley line identified as G1* in WO2009/021285 were grown in the glasshouse to produce F6 progeny. F6 plants were then grown in the field to produce F7 progeny. To combine the Hor2-lys3a mutations with the Hor3 null mutation, plants of the F7 generation were crossed with the D-hordein negative BC₂ plants derived from Ethiopia R118 and the F1 progeny selfed to produce F2 seeds. F2 seeds were cut in half and the germ-half germinated and the seedlings screened by B- and D-hordein PCR and for gamma-hordein as described in WO2014/197943. The remaining half of each seed comprising the endosperm was ground in a solution containing 8M urea, 1% DTT and the extracted proteins separated by SDS-PAGE. The absence of characteristic protein bands at approximately 50 kDa indicating the absence of the C-hor proteins. Three hordein triple-nulls, designated T1, T2, and T3 were identified from about 300 F2 seeds. The expected frequency for the combination of three recessive mutations, each in the homozygous state, by Mendelian genetics was 1/64, presuming that the Hor2 (B-hordeins) and Hor3 (D-hordeins) loci are separated far enough on chromosome 1H to recombine readily.

The three plants which were homozygous for each of the three mutations (Hor2-lys3a-Hor2) designated T1, T2 and T3 were maintained and propagated through up to three generations of single-seed descent, selecting the 12 heaviest seeds in each generation. Average seed weights of F3 seeds from these lines were: T1, 38.2 mg; T2, 37.0 mg; T3, 39 mg. Seed yield per line (grams of seed per 20 heads) and plant heights were measured. Plants which produced poorly filled heads were discarded. Two F4 lines were selected: T2-4-8 and T2-6-A5 and further trialed in the field. Of these, T2-4-8 was selected as having slightly better grain yield and designated as barley ULG3.0.

An important phenotype for barley grain, related to grain size and shape and an indicator of grain yield, is the percentage of grains which do not pass through sieves with a mesh size of 2.8, 2.5, 2.2 and 2.0 mm, in particular the 2.8 mm sieve. Smaller grain makes the processing and malting less efficient relative to wild-type barley. This phenotype is referred to as “2.8 mm screenings” and is indicated as the percentage of grains that do not pass through the particular sieve. For wild-type cultivars such as Sloop, the 2.8 mm screening parameter is typically 95-98%. For hordein-deficient lines such as the Hor2-lys3a double mutant (ULG2.0), the 2.8 mm screening parameter was generally about 53%. Sometimes, depending on the growth conditions e.g. drought, it was less than 10% and the majority of grains could pass through the 2.5 mm sieve. For ULG3.0, the 2.8 mm screening parameter was about 54%. Average weights (mg) of field grown grains were: Sloop, 53.6+/−0.9, ULG2.0, 33.5+/−0.4; ULG3.0, 39.1+/−0.3. ULG3.0 therefore provided 69% of the grain yield compared to ULG2.0 at 50% relative to the wild-type Sloop (100%). ULG3.0 therefore represented a substantial improvement in grain yield compared to the ULG2.0 line. However, the 2.8 mm screening parameter remained a problem for barley ULG3.0.

### Example 3. Generation of a Further Barley Mutants with Increased Yield

Although the ULG3.0 barley line produced as described in Example 2 had an increased grain yield compared to ULG2.0, it was still desirable to increase that further. Therefore, plants of ULG3.0 were crossed with plants of wild-type cultivars Sloop, Baudin and Yagan, and with plants of hordein triple null lines identified containing 50% of each parent germplasm. These hordein triple-null lines were intercrossed, and also crossed to wild-type cultivars Hindmarsh and Commander. Progeny comprising all three null mutations were backcrossed twice to plants of Sloop, Baudin, Hindmarsh and Commander and more homozygous lines produced by single-seed descent. One resultant line of the many that were produced was selected and designated as barley ULG3.1.

From the intercrosses with the Sloop, Yagan and Baudin plants, a second round of intercrosses was performed to combine the genetic backgrounds of all three parent cultivars starting with plants which each comprised the triple null mutations. From the intercrossed F1 plants, all of the progeny were expected to comprise all three mutations. About 1000 F2 seed per pedigree were planted in rows in the field, and F2 plants selected which were relatively shorter and produced F3 seed that were larger, with well-filled heads. Both early and late maturing plants were selected. In a following generation grown in the field, lines were selected which additionally exhibited relatively high grain amylase, relatively high harvest index and head length, optimal height (semi-dwarf), lack of lodging and disease resistance to powdery mildew. From the 1500 families, about 20 of the best were selected. Data for the 20 best lines is provided in Table 1. Individual seed weights (kernel weight) were improved beyond that of ULG3.0 of 41.8 mg/seed with the highest seed weight of 48.4 mg/seed being observed for line P12072-2. This improvement in seed size was accompanied by increased harvest index, a measure of efficiency of seed formation, above 40% with the highest harvest index of 46.5% being measured in line P12124-1. Most importantly, the percentage 2.8 mm screenings also improved from 53.5% for ULG3.0 to over 80% with a high of 97.3% for line P12140-1.

One selected line was fixed by a single-seed generation to produce plants that were homozygous for the three null alleles at Hor2-lys3a-Hor3 and designated ULG3.2. The 2.8 mm screening parameter for ULG3.2 was in the range of 80-93% in several replications when grown in the field compared to about 97% for Sloop, 85% for Hindmarsh, 96% for wild-type cultivar Oxford, and 98% for Maratime.

Average seed weights and thicknesses were: ULG2.0, 33.4 mg, 2.4 mm; ULG3.0, 41.8 mg, 2.5 mm; ULG3.2, 47.2 mg, 2.8 mm.

TABLE 1
Second selection of ULG 3.2 lines.

| Plant ID | Mean | Mean | Harvest | No of | Kernel | % 2.8 mm |
|---|---|---|---|---|---|---|
| No | tiller | head | index (%) | Tillers | weight | Screening |
| P12072-2 | 84.7 | 8.3 | 39.0% | 9 | 48.4 | 88.3 |
| P12132-1 | 83.0 | 8.2 | 43.9% | 5 | 47.5 | 92.7 |
| P12048-1 | 75.0 | 7.0 | 39.4% | 8 | 47.2 | 86.7 |
| P12122-2 | 74.3 | 6.7 | 35.0% | 5 | 47.2 | 96.5 |
| P12049-1 | 70.0 | 6.3 | 38.7% | 12 | 46.5 | 93.6 |
| P12125-1 | 78.0 | 7.7 | 42.0% | 10 | 46.2 | 95.3 |
| P12140-1 | 105.7 | 9.0 | 32.4% | 15 | 45.9 | 97.3 |
| P12055-2 | 57.0 | 5.8 | 36.3% | 5 | 45.7 | 91.9 |
| P12088-2 | 72.7 | 6.3 | 42.6% | 8 | 45.4 | 90.7 |
| P12125-2 | 71.7 | 5.3 | 45.3% | 8 | 45.0 | 95.0 |
| P12043-2 | 86.7 | 9.0 | 29.5% | 12 | 44.7 | 85.4 |
| P12050-1 | 64.3 | 6.0 | 36.5% | 4 | 44.4 | 93.6 |
| P12148-1 | 93.3 | 7.3 | 35.0% | 14 | 44.2 | 90.0 |
| P12149-1 | 103.3 | 8.0 | 34.7% | 15 | 44.0 | 91.2 |
| P12124-1 | 79.3 | 7.3 | 46.5% | 8 | 43.7 | 96.8 |
| P12152-2 | 98.3 | 9.0 | 34.8% | 9 | 43.5 | 91.5 |
| P12049-3 | 76.3 | 6.3 | 40.0% | 7 | 43.2 | 92.4 |
| P12100-2 | 79.3 | 6.3 | 46.5% | 15 | 43.2 | 88.3 |
| P12126-1 | 73.0 | 6.0 | 37.0% | 10 | 43.1 | 98.0 |
| P12122-1 | 64.7 | 6.0 | 45.4% | 5 | 42.8 | 90.8 |
| P12048-2 | 65.0 | 5.7 | 41.8% | 4 | 42.5 | 93.9 |
| P12148-2 | 90.3 | 6.3 | 33.3% | 7 | 42.5 | 94.9 |
| P12159-2 | 82.7 | 7.7 | 33.6% | 12 | 42.4 | 83.5 |
| P12139-1 | 113.7 | 8.3 | 33.9% | 8 | 42.2 | 88.1 |
| P12159-1 | 96.3 | 8.8 | 31.9% | 14 | 41.7 | 94.1 |
| P12139-2 | 89.3 | 7.7 | 34.4% | 5 | 41.3 | 96.8 |
| P12064-2 | 82.3 | 7.2 | 40.8% | 11 | 41.2 | 92.4 |
| P12120-2 | 75.0 | 6.8 | 39.6% | 18 | 41.0 | 84.0 |
| Selection | | >4 < 10 | >35% | 5 > 20 | >40 | >80% |
| criterion | | | | | | |

### Example 4. Measurement of Hordein Levels in Barley Mutants

### Determination of Hordein Content of ULG3.0 Flour by Multiple Reaction Monitoring Mass Spectrometry (MRM MS)

To measure the hordein content accurately, the MRM MS assay was used, as follows. Grains or half-grains were milled to produce flour which, as wholemeal flour, had the same composition as the entire grain. The prolamin polypeptides from 20 mg flour samples were extracted using 200 μL of a solution containing 55% (v/v) isopropanol and 2% (w/v) dithiothreitol (DTT). An aliquot of the extract, equivalent to 5 mg flour, was subjected to buffer exchange into 8M urea in 0.1 M Tris-HCl, pH 8.5, by centrifuging thrice, using a 10 kDa MW cut-off filter unit. The cysteines in the polypeptides were alkylated by addition of 100 μL of 50 mM iodoacetamide and incubation for 1 hr at room temperature. The buffer was exchanged to 100 μL of 50 mM ammonium bicarbonate, pH 8.5, and polypeptides digested with 10 μL (20 μg) of trypsin for 18 hr at 37° C. The peptides were collected by filtration through the 10 kDa filter, dried and reconstituted in 30 μL of 1% (v/v) formic acid. Peptides were separated by liquid chromatography on a Shimadzu Nexera HPLC with Phenomemenex column (Kinetex, 1.7 μm, C18, 100×2.1 mm) with a gradient from 5% B to 40% B over 10 min at a flow rate of 0.4 mL/min. Solvent A was 0.1% (v/v) aqueous formic acid, solvent B was 90% (v/v) acetonitrile containing 0.1% (v/v) formic acid. The HPLC eluate was directly coupled to the mass spectrometer and MRM analysis was performed on a 4000 QTRAP mass spectrometer targeting hordein-derived tryptic peptides. Data was analysed using Analyst v1.5 software and MultiQuant v2.0.2 software using (peak area integration).

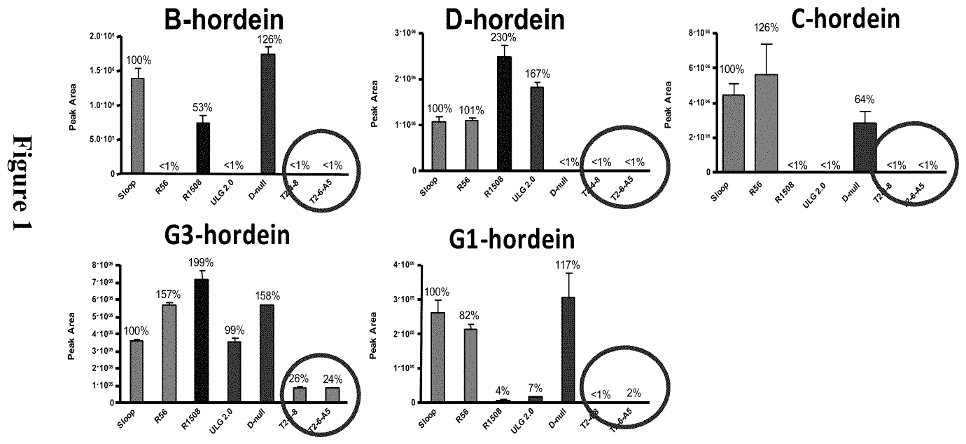

FIG. 1 shows the data obtained for selected B-hordeins, C-hordein, D-hordein, gamma-3-hordein (G3) and gamma-1-hordein (G1). FIG. 1 presents the mean peak area for each peptide MRM transition normalized to the level in Sloop (100%), for four replicate injections from each half-grain from control barley (wild-type, cv Sloop), hordein single-null lines: Risø 56, Risø 1508 and the D-null line derived from Ethiopia R118, the hordein double-null line ULG2.0 and the triple-null lines T2-4-8 and T2 A5 (circled). One prototypic peptide was chosen to represent each hordein family, namely: for B-hordein, TLPTMCSVNVPLYR (SEQ ID NO: 48); for D-hordein, DVSPECRPVALSQVVR (SEQ ID NO: 49); for C-hordein, LPQKPFPVQQPF (SEQ ID NO: 50); for G3-hordein, QQCCQQLANINEQSR (SEQ ID NO: 50) and for G1-hordein, CTAIDSIVHAIFMQQGR (SEQ ID NO: 51). These peptides appear frequently and at relatively high abundance in wild-type barley, and were chosen on that basis.

It was seen that the triple-null ULG3.0 grain from line T2-4-8 and second triple-null line T2-6-A5 did not have detectable levels of B-, C-, D-, or, most surprisingly, gamma-1-hordein. That is, less than 1% of the level was observed relative to wild-type. In the same way, gamma-2-hordein was not detected in the ULG3.0 grain. There was a relatively low level of gamma-3-hordein (circled), present at a level of about 20% compared to the level of G3 in Sloop. Gamma-3 hordein is a minor hordein; the gamma-3 hordein content of Sloop is much less than 1% of the total hordein content. The single-null and double-null grains did not accumulate the appropriate hordein, e.g. RisØ56 and ULG2.0 did not accumulate B-hordeins as expected, and Risø1508 and ULG2.0 did not accumulate C-hordeins as expected. The D-null grain exhibited wild-type levels of B- and C-hordeins but did not accumulate D-hordein.

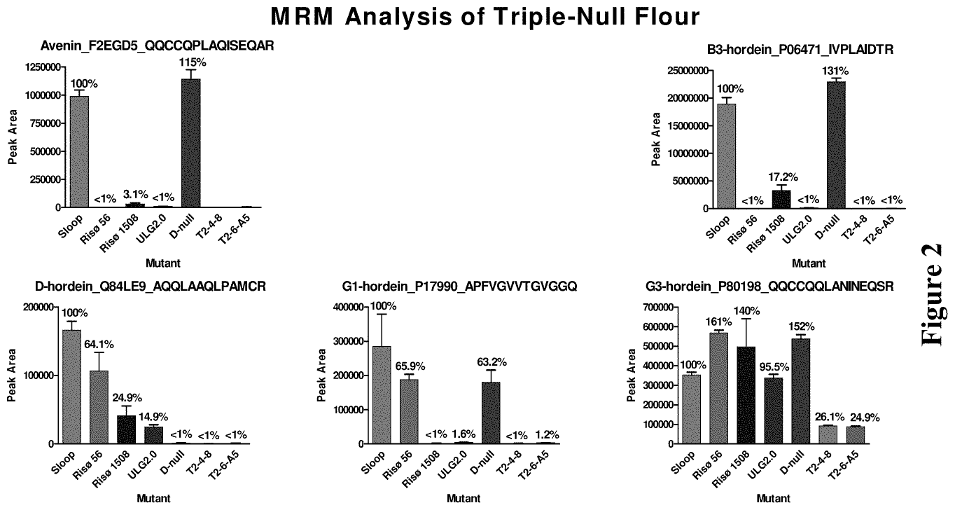

When the analysis was repeated using several different peptide sequences, in particular the D-hordein peptide AQQLAAQLPAMCR (SEQ ID NO: 85) which is present in the wild-type D-hordein protein towards the C-terminus, well after the position of the stop codon in the B-hordein mutant, similar results were obtained (FIG. 2). The avenin-like A protein was also absent from the flour.

It was clear that grain obtained from the hordein triple nulls T2-4-8 and T2-6-A5 did not contain detectable levels of B-, C-, D-hordeins and selected gamma-1 (P17990) and gamma-2 hordeins. The observation for the gamma-1 and gamma-2 hordeins was most unexpected to the inventors, as the triple null mutant lines were not known to contain any mutations that would entirely silence the corresponding genes.

The low hordein content of the ULG3.0 grain as determined by MRM was confirmed by a two-dimensional gel electrophoresis method, as follows. Fifty μg of alcohol soluble protein from extracts of flour from each of hordein null lines T2-4-8 and T2-6-A5 as well as control barley cv Risø 56, each spiked with 1 μg of the landmark polypeptide standards BSA, soy trypsin inhibitor and horse myoglobin were stained with 0.006% (w/v) Colloidal Coomassie G250 according to Tanner et al. (2013) and compared to standard proteins of 20, 30, 40, 50, 60, 80, and kDa (M; Benchmark Protein Ladder, Invitrogen). Spots were cut out of the 2D gel and the following proteins from the control Risø 56 were identified by mass spectrometry of tryptic peptides: C-hordein, gamma-2-hordein (γ2), gamma-3-hordein (γ3) and gamma-1-hordein (γ1), The predicted positions of gamma-1-, gamma-2, and gamma-3-hordeins in the gels from the ULG3.0 grain were identified by comparison with the Risø 56 gel. Only gamma-3-hordein was observed in ULG3.0 flour, the other three polypeptides were not detected. The gamma-3 hordein concentration of each spot were measured by three methods: 1) As a percentage of all spot volumes from the 50 μg of protein: ULG3.0 average γ3 content was 13.5±1.6 ppm; 2) Relative to the spot intensity of 1 μg of BSA: ULG3.0 average γ3 content was 10.9±1.3 ppm; 3) Relative to the spot volume (intensity×area) of 1 μg of BSA: ULG3.0 average γ3 content was 3.4±0.41 ppm.

The low hordein content of the ULG3.0 grain as determined by MRM was further confirmed by an ELISA method, as follows. Twenty mg of wholemeal flour samples or the endosperm half of grains were crushed and washed thrice in 0.5 ml of MilliQ water by shaking at 30/sec for 3×30 sec in a 96 well Vibration Mill (Retsch Gmbh, Rheinische) and centrifuged at 14,000 rpm for 5 min. Prolamins in the flours were extracted into an alcoholic solution consisting of 0.5 ml of 50% (v/v) isopropanol/1% (w/v) DTT, for the control lines Sloop, Risø56, Risø1508, and for ULG2.0, the hordein triple-null lines T1, T2, and T3, and the single seed descent progeny from T2-4-8 and T2-6-A5. Protein concentrations were determined according to Bradford (1976) and 40 ng (1900 ng for the triple-null grains) of alcohol soluble protein diluted with a solution containing ELISA systems diluents with a constant excess of 0.2 mM H₂O₂ added to quench any DTT remaining from the initial extract. Diluted protein solutions were added to ELISA plate wells (ELISASystems, Windsor, Queensland, Australia), washed and developed at 37° C. for 15 minutes according to the manufacturer's instructions. The amount of hordein in the control extracts was calibrated against a standard of 0-50 ng of Sloop total hordein. Hordein content of the triple-nulls was calibrated against a standard of 0-5 ng of ULG2.0 total hordein. The Sloop and ULG2.0 hordeins were prepared as described by Tanner et al. (2010).

By the ELISA method, the total hordein content of double-null flour samples was 2.9% relative to the wild-type cv Sloop, whereas the remaining hordein content of the two selected hordein triple-null lines, T2-4-8 and T2-6-A5 were 3.9 and 1.5 ug/g (parts per million, ppm; Table 2) both significantly below the FSANZ legislated level of 20 ppm for gluten in gluten free food and approximately 15,000 fold lower than in the wild-type cv Sloop grain.

TABLE 2
Summary of hordein content of hordein
single-, double- and triple-null lines.

| Line | mg Hordein/gm flour | % of Sloop |
|---|---|---|
| Sloop | 56.6 ± 3.3 | 100% |
| Risø56 | 33.3 ± 1.1 | 58.8% |
| Risø1508 | 4.9 ± 0.26 | 8.7% |
| ULG2.0 | 1.67 ± 0.07 | 2.9% |
| T2-4-8 (ULG3.0) | 0.0039 ± 0.0017 | 0.007% |
| T2-6-A5 | 0.0015 ± 0.0004 | 0.0027% |

### Determination of Hordein Content of ULG3.1 and ULG3.2 Flours by MRM MS

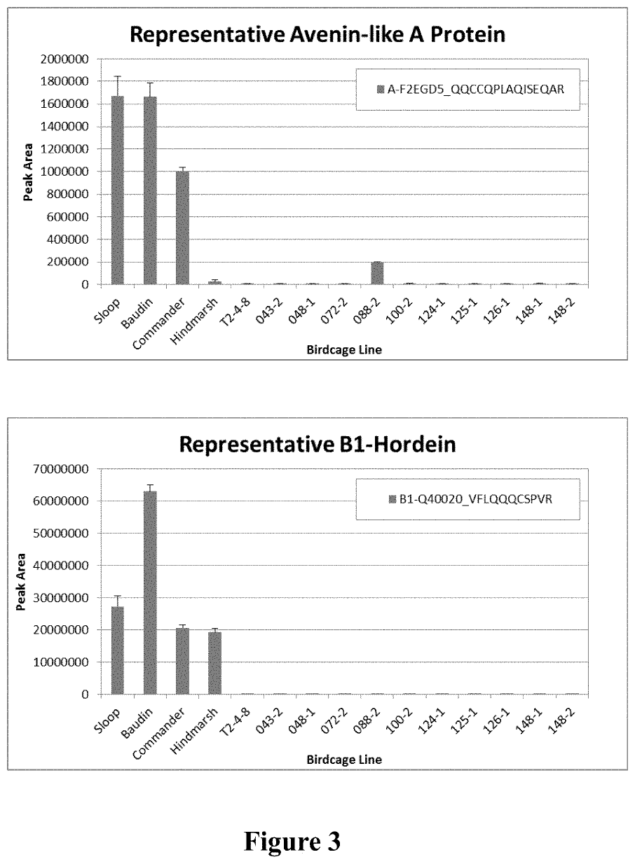

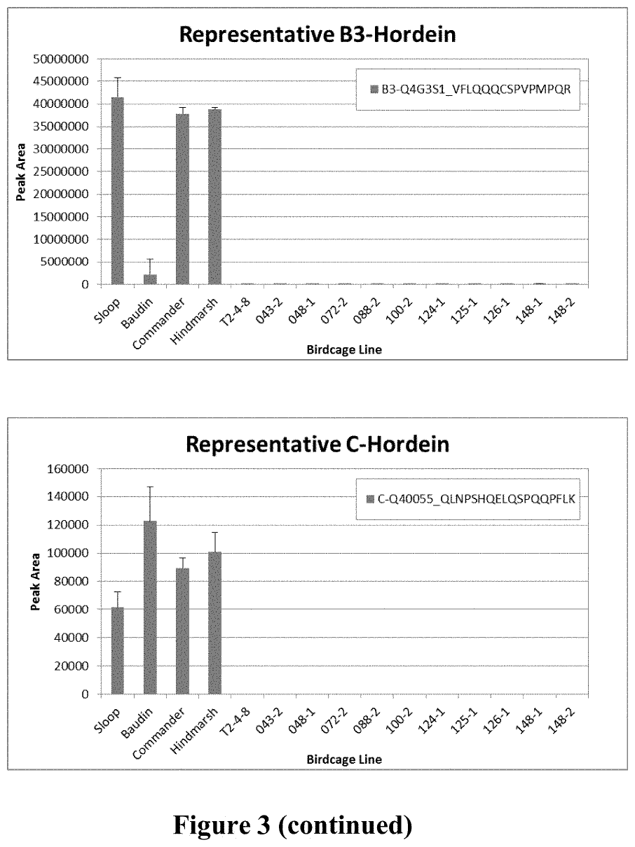

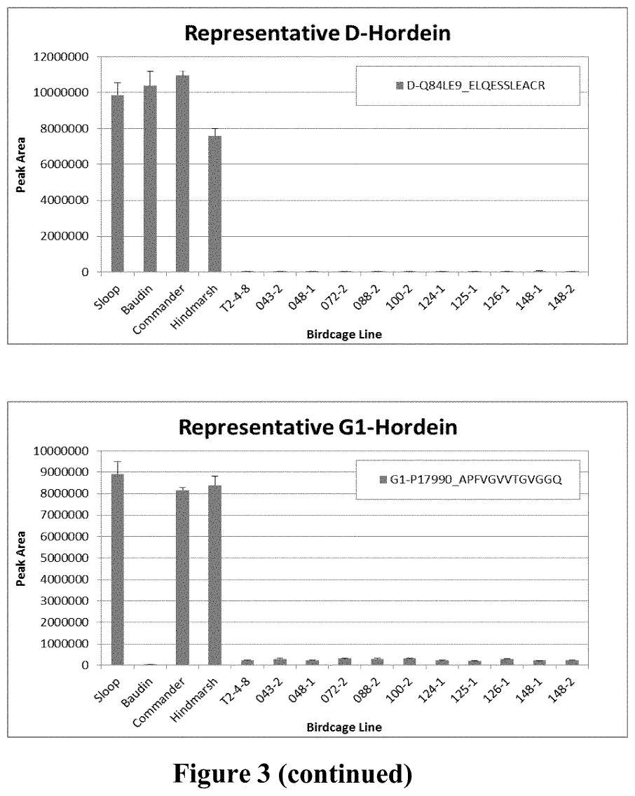

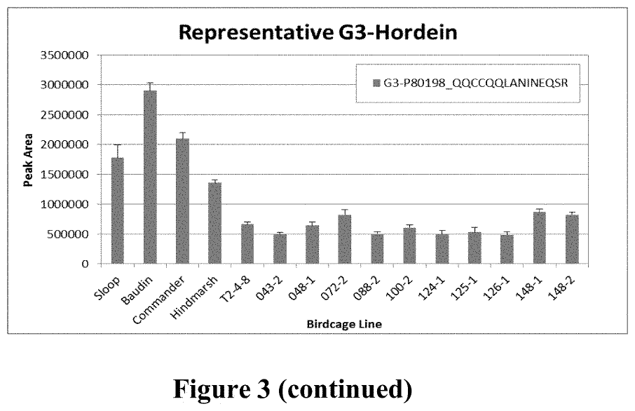

The hordein content of flour milled from grains of the ULG3.1 line and 10 candidate lines for ULG3.2 was determined by MRM MS as described for the ULG3.0 grain. Half-grains were milled to flour and the prolamin proteins from 20 mg flour (n=4 replicates) were extracted, alkylated, trypsin digested and analysed by MRM MS as described above. The data are plotted in FIG. 3, which show the mean peak area for each peptide (sum of three MRM transitions) from each half-grain of ULG3.1 and lines arising from the double parental intercross lines designated 043-2-148-2. These are plotted in comparison with control barley (wild-type cultivars Sloop, Baudin, Commander and Hindmarsh) and the triple-null line T2-4-8. The peak area is shown for a selected prototypic peptide, representative of each hordein family, Uniprot accession and amino acid sequences as follows:

(SEQ ID NO: 52; from F2EGD5, central
to avenin-like A protein)
A-F2EGD5_QQCCQPLAQISEQAR
(SEQ ID NO: 53; close to the
N-terminus of B1-hordein)
B1-Q40020_VFLQQQCSPVR
(SEQ ID NO: 54)
B3-Q4G3S1_VFLQQQCSPVPMPQR
(SEQ ID NO: 55; close to the
N-terminus of C-hordein)
C-Q40055_QLNPSHQELQSPQQPFLK
(SEQ ID NO: 56; from Q84LE9,
before the stop codon at Y150)
D-Q84LE9_ELQESSLEACR
(SEQ ID NO: 57; from P17990,
C-terminal peptide)
G1-P17990_APFVGVVTGVGGQ,
and
(SEQ ID NO: 58; from P80198,
central to γ3-hordein)
G3-P80198_QQCCQQLANINEQSR.

The level of D-hordein in the bi-parental intercross grains shown in FIG. 3 was similar to that in grain from ULG2.0 and the hordein triple null lines T2-4-8 and T2-6-A5, near zero, confirming the observation by 2D PAGE that D-hordein was not detected in grains from the T2-4-8 and T2-6-A5 lines. The level of gamma-1-hordein in these bi-parental intercrossed grain was also similar to that in ULG2.0 and the hordein triple null lines T2-4-8 and T2-6-A5. Several ULG3.2 lines had near zero level of the peptide APFVGVVTGVGGQ (SEQ ID NO: 59). Gamma-1-hordein was not detected by 2D PAGE of T2-4-8 and T2-6-A5 lines. The level of gamma-3-hordein in the bi-parental intercross grains was also similar to that in ULG2.0 and the hordein triple null lines except for line 124.1 (ULG3.2) in which it was very low. Gamma-3-hordein was also detected at reduced levels by 2D PAGE of the T2-4-8 and T2-6-A5 lines.

Interestingly, a synergistic effect was seen of the Hor2 and lys3a mutations in reducing the D-hordein content in ULG2.0 even though no Hor3 mutation (D-hordein) was present. In a similar fashion, the presence of all three mutations (Hor2-lys3a-Hor3) had a synergistic effect on reducing accumulation of the gamma-1- and gamma-2-hordeins as defined by the peptides above.

### Example 5. Characterisation of ATIs in Barley Flour from Wild-Type Varieties

The two-step total protein extraction protocol from selectively bred barley lines was performed as described in Example 1. To remove any type of bias resulting from the biological differences between cultivars, a MRM-based experiment was performed in order to identify the relative amount of ATI complement of three wild-type barley varieties: cv Sloop, Hindmarsh, and Commander.

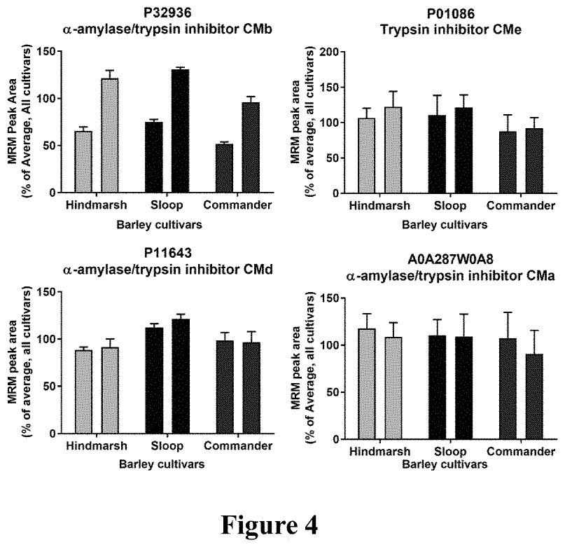

As shown in Table 3, two prototypic peptides were chosen to represent each CM protein family. These peptides appear frequently and at relatively high abundance in wild-type barley and were chosen on the basis of high intensity and lack of interferences. The results showed that the three wild-type cultivars possess similar amounts of ATIs (FIG. 4). Hindmarsh is widely known as a food-grade barley while cv Sloop and Commander are commonly used for malting purposes. The extraction protocol employed IPA/DTT to preferentially solubilise gluten and co-extracting ATIs; followed by a urea-based buffer, to extract ATIs from the grain extract. Using LC-MS/MS the three exemplar barley varieties (cv Sloop, Hindmarsh and Commander) were analysed for peptides representative of alpha amylase trypsin inhibitors CMb (P32936), CMd (P11643) CMa (A0A287W0A8) and the trypsin inhibitor CMe (P01086). It was seen that there were no significant differences in expression of these proteins and peptides between the selected wild-type varieties. FIG. 4 presents the mean peak area for each peptide MRM transition normalized to the level in all three cultivars (100%), for four replicate injections from each flour derived from control barley wild-type, cv Sloop, and commercial lines Hindmarsh and Commander.

### Example 6. Phylogenetic Tree of Identified Proteins

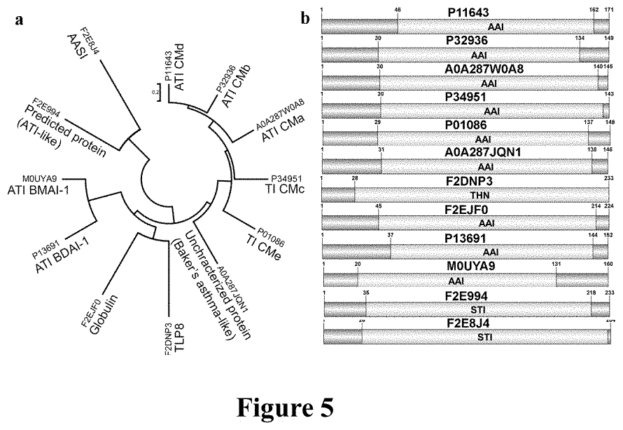

A phylogenetic tree (FIG. 5a) was constructed using the data obtained from the protein alignment described in Example 1. The 12 ATI-like protein sequences identified in this Example and 17 ATI-like protein sequences obtained from the Uniprot database were aligned by using MUSCLE (http: http://www.ebi.ac.uk/Tools/msa/muscle/), and subsequently phylogenetic analysis was performed in MEGA X software as described in Kumar et al. (2018), using the neighbour-joining method according to Saitou et al. (1987). Using this strategy, 6 proteins matched to ATIs were identified, a further 2 trypsin inhibitors, and 4 proteins that were not annotated as ATIs, but shared significant homology or contained alpha-amylase/trypsin inhibitory domains. These included a predicted protein (with a soybean trypsin inhibitor domain), thaumatin, bifunctional alpha-amylase/subtilisin inhibitor and a globulin. These proteins contain a domain that may be responsible for an innate inflammatory reaction as such these four proteins were included in further analyses, including the quantitative LC-MS experiments of Example 7. The inhibitory domain was either an alpha-amylase inhibitor (AAI), soybean trypsin inhibitor (STI) or pathogenesis-related (PR). Globulin (F2EJF0) is primarily known as a seed storage protein, has an alpha-amylase inhibitor (AAI) domain and bifunctional inhibitor/plant lipid transfer protein/seed storage helical domain which are involved in endopeptidase inhibitory activity (Samuel et al., 2002; Strobl et al., 1998). The barley alpha-amylase/subtilisin inhibitor (BASI) shares similar function to ATIs and plays a role in plant defense by inhibiting subtilisin-type serine proteases of pathogens and pests (Mundy et al., 1983; Nielsen et al., 2004). Thaumatin is referred to as pathogenesis-related group 5 (PR5), as many thaumatin-like proteins accumulate in plants in response to infection by a pathogen and possess antifungal activity (Ruiz-Medrano et al., 1992).

The phylogenetic tree in FIG. 5 shows the ATI protein clusters; alpha-amylase/trypsin inhibitor CMd (ATI CMd), alpha-amylase/trypsin inhibitor CMb (ATI CMb), alpha-amylase/trypsin inhibitor CMa (ATI CMa), trypsin inhibitor CMc (TI CMc), trypsin inhibitor CMe (TI CMe), uncharacterized protein (similar to Baker's asthma allergen (BDP)), thaumatin-like protein 8 (TLP8), globulin, alpha-amylase inhibitor BDAI (ATI BDAI-1), alpha-amylase inhibitor BMAI (ATI BMAI-1), predicted protein (ATI-like), alpha-amylase/subtilisin inhibitor (AASI).

CM proteins are encoded by multi-gene families and members of this protein family are differentially expressed in various species (Lazaro et al., 1985). These proteins show conservation of the multiple cysteine residues (FIG. 5c) even though they are encoded by loci on 4 different chromosomes in the barley genome. The chromosomal location of genes in this protein family has been suggested to influence a protein's inhibitory properties (Barber et al., 1986). For example, genes for CMb and CMd protein are located on chromosome 4, and has shown no inhibitory activity (Barber et al., 1986). However, this has not been demonstrated conclusively. All selected peptides were fully tryptic, without any unusual modifications, cysteines were present as carbomidomethyl cysteine, and common modifications (oxidized Met; pyroglutamination Gln/Cys) were also monitored. In total, 54 peptides were monitored across 12 proteins (Table 3). Peptide sequences were subjected to homology searching (BLASTp) against both UniProt and NCBI databases to assess the uniqueness of each peptide marker. Many of the identified peptides were noted to be common to multiple protein isoforms, but are representative of specific ATI classes.

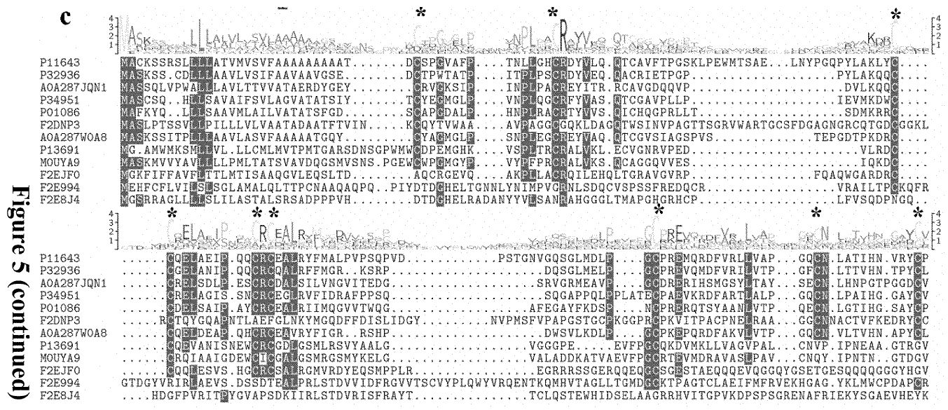

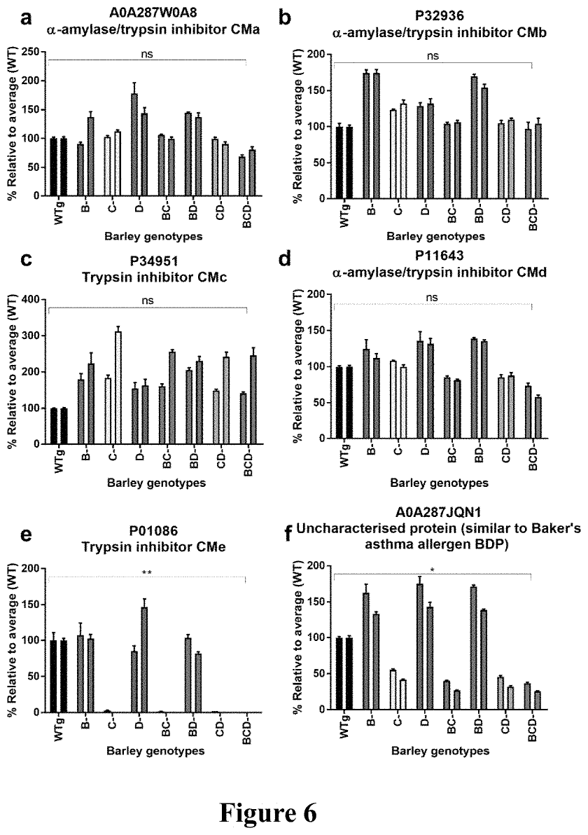

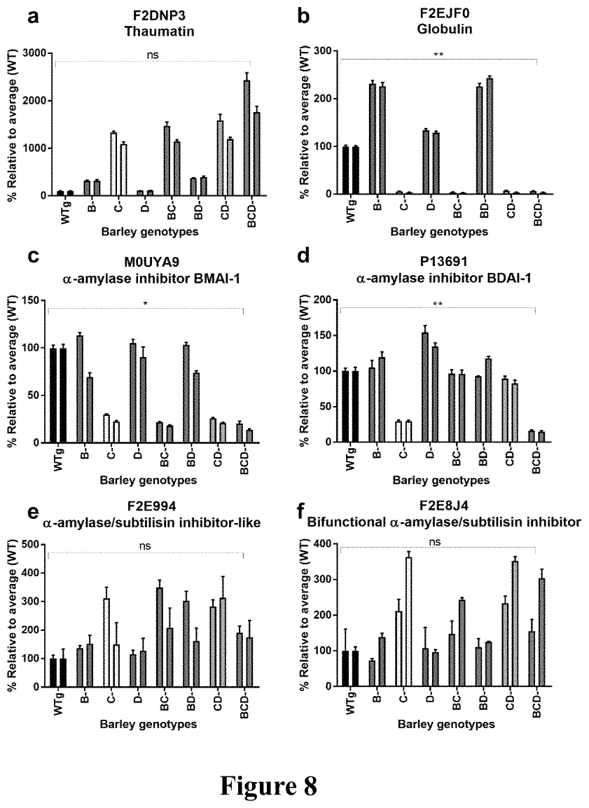

TABLE 3
ATI peptides monitored by LC-MRM-MS. Peptides used in FIG. 6 and 8
are annotated by an asterisk.

| Uniprot | | | Unique to |
|---|---|---|---|
| accession | Protein name | Peptide sequence | H. vulgare |
| P11643 | Alpha-amylase/trypsin | DYVLQQTCAVFTPGSK* | Yes |
| | inhibitor CMd | (SEQ ID NO: 98) | |
| | | LLVAPGQCNLATIHNVR* | No |
| | | (SEQ ID NO: 99) | |
| | | LYCCQELAEIPQQCR | No |
| | | (SEQ ID NO: 100) | |
| P13691 | Alpha-amylase | CGDLGSMLR* (SEQ ID NO: 101) | No |
| | inhibitor BDAI-1 | DCCQEVANISNEWCR | Yes |
| | | (SEQ ID NO: 102) | |
| | | LECVGNR (SEQ ID NO: 103) | Yes |
| | | LLVAGVPALCNVPIPNEAAGTR* | Yes |
| | | (SEQ ID NO: 104) | |
| | | SVYAALGVGGGPEEVFPGCQK | Yes |
| | | (SEQ ID NO: 105) | |
| | | VPEDVLR (SEQ ID NO: 106) | Yes |
| P32936 | Alpha-amylase/trypsin | DYVEQQACR* (SEQ ID NO: 107) | No |
| | inhibitor CMb | EVQMDFVR* (SEQ ID NO: 108) | No |
| | | IETPGPPYLAK (SEQ ID NO: 109) | No |
| | | QQCCGELANIPQQCR | No |
| | | (SEQ ID NO: 110) | |
| | | SRPDQSGLMELPGCPR | No |
| | | (SEQ ID NO: 111) | |
| P34951 | Trypsin inhibitor CMc | AFPPSQSQGAPPQLPPLATECPAEV | Yes |
| | | K(SEQ ID NO: 112) | |
| | | ELAGISSNCR* (SEQ ID NO: 113) | Yes |
| | | FYVASQTCGAVPLLPIEVMK* | Yes |
| | | (SEQ ID NO: 114) | |
| P01086 | Trypsin inhibitor CMe | CCDELSAIPAYCR (SEQ ID NO: 115) | Yes |
| | | DSPNCPR* (SEQ ID NO: 116) | Yes |
| | | LLTSDMK* (SEQ ID NO: 117) | Yes |
| | | TYVVSQICHQGPR (SEQ ID NO: 118) | Yes |
| A0A287W0A8 | Alpha-amylase/trypsin | CCQELDEAPQHCR | Yes |
| | inhibitor CMa | (SEQ ID NO: 119) | |
| | | EYVAQQTCGVSIAGSPVSTEPGDTP | Yes |
| | | K*(SEQ ID NO: 120) | |
| | | SHPDWSVLK* (SEQ ID NO: 121) | Yes |
| A0A287JQN1 | Uncharacterised | CAVGDQQVPDVLK | Yes |
| | protein | (SEQ ID NO: 122) | |
| | (similar to Baker's | CDALSILVNGVITEDGSR | Yes |
| | asthma allergen BDP) | (SEQ ID NO: 123) | |
| | | DYGEYCR (SEQ ID NO: 124) | Yes |
| | | ELSDLPESCR* (SEQ ID NO: 125) | Yes |
| | | MEAVPGCDR (SEQ ID NO: 126) | Yes |
| | | SIPINPLPACR* (SEQ ID NO: 127) | Yes |
| M0UYA9 | Alpha-amylase | SQCAGGQVVESIQK* | Yes |
| | inhibitor BMAI-1 | (SEQ ID NO: 128) | |
| | | ATVAEVFPGCR* (SEQ ID NO: 129) | Yes |
| | | ELGVALADDK (SEQ ID NO: 130) | Yes |
| | | QIAAIGDEWCICGALGSMR | Yes |
| | | (SEQ ID NO: 131) | |
| F2E994 | Predicted protein | DLVLLDYAGR* (SEQ ID NO: 132) | Yes |
| | (ATI-like) | EPLVVVFK* (SEQ ID NO: 133) | No |
| | | LSTDVVIDFR (SEQ ID NO: 134) | Yes |
| F2DNP3 | Thaumatin-like protein | AAGGCNNACTVFK | No |
| | TLP8 | (SEQ ID NO: 135) | |
| | | CTQYGQAPNTLAEFGLNK | No |
| | | (SEQ ID NO: 136) | |
| | | CQYTVWAAAVPAGGGQK | Yes |
| | | (SEQ ID NO: 137) | |
| | | LDAGQTWSINVPAGTTSGR (SEQ ID | Yes |
| | | NO: 138) | |
| | | TGCSFDGAGNGR* (SEQ ID NO: 139) | No |
| | | VITPACPNELR*(SEQ ID NO: 140) | Yes |
| F2E8J4 | Alpha- | ADANYYVLSANR (SEQ ID NO: 141) | Yes |
| | amylase/subtilisin | AYTTCLQSTEWHIDSELAAGR | Yes |
| | inhibitor | (SEQ ID NO: 142) | |
| | | GGAWFLGATEPYHVVVFK | No |
| | | (SEQ ID NO: 143) | |
| | | HCPLFVSQDPNGQHDGFPVR | Yes |
| | | (SEQ ID NO: 144) | |
| | | HVITGPVK (SEQ ID NO: 145) | Yes |
| | | ITPYGVAPSDK (SEQ ID NO: 146) | Yes |
| | | SADPPPVHDTDGHELR* | Yes |
| | | (SEQ ID NO: 147) | |
| | | YSGAEVHEYK* (SEQ ID NO: 148) | No |
| F2EJF0 | Predicted protein | DYEQSMPPLR* (SEQ ID NO: 149) | No |
| | (Globulin) | QILEHQLTGR* (SEQ ID NO: 150) | Yes |
| | | QQQGEGFSGEGAQQKPK | No |
| | | (SEQ ID NO: 151) | |

### Example 7. Measurement of ATI Levels in Mutant Barley Grain

LC-MRM-MS analysis was conducted yielding the peak area for each peptide as described in Example 1 and in Bose et al. (2019). Peak area for each peptide was compared between barley varieties or lines. All data were converted to a percentage relative to the average peak area of WT barley cv Sloop for ease of data interpretation as the peak area is directly related to the peptide sequence which dictates the ionisation efficiency.

The results of relative quantitation of selected peptides from ATIs and ATI-related proteins across the eight barley lines are presented in FIGS. 6 and 8. The ATI peptides were selected using the criteria as follows: (1) derived from ATI proteins; (2) fully tryptic; (3) identified with ≥95% confidence; and (4) contained no unusual/variable modifications, such as deamidation. The peptide list was then sorted by peak signal intensity to yield the high responding peptides.

The sequences of the ATI proteins that were measured are shown below.

The sequences of the ATIs that were identified as being reduced in the barley mutant lines described herein are shown below (underlined peptides are those that were measured by LC-MRM-MS):

>P01086- Trypsin inhibitor CMe (SEQ ID NO: 86):
MAFKYQLLLSAAVMLAILVATATSFGDSCAPGDALPHNPLRACRTYVVSQICH
QGPRLLTSDMKRRCCDELSAIPAYCRCEALRIIMQGVVTWQGAFEGAYFKDSP
NCPRERQTSYAANLVTPQECNLGTIHGSAYCPELQPGYGVVL
>F2EJF0- Globulin (SEQ ID NO: 87):
MGKFIFFAVFLTTLMTISAAQGVLEQSLTDAQCRGEVQAKPLLACRQILEHQLT
GRAVGVRPFQAQWGARDRCCQQLESVSHGCRCSALRGMVRDYEQSMPPLRE
GRRRSSGERQQEQGCSGESTAEQQQEVQGGQYGSETGESQQQQGGGYHGVTV
GRGGQQQGQMLCRERPQRQQQGEGFSGEGAQQKPKVGRVRLTKVRLPTACRI
EPQECSVFSTLPVLG
>M0UYA9- Alpha-amylase inhibitor BMAI-1 (SEQ ID NO: 88):
MASKMVVYAVLLLLPMLTATSVAVDQGSMVSNSPGEWCWPGMGYPVYPFPR
CRALVKSQCAGGQVVESIQKDCCRQIAAIGDEWCICGALGSMRGSMYKELGV
ALADDKATVAEVFPGCRTEVMDRAVASLPAVCNQYIPNTNGTDGVCYWLSY
YQPPRQMSSR
>P13691- Alpha-amylase inhibitor BDAI-1 (SEQ ID NO: 89):
MGAMWMKSMLLVLLLCMLMVTPMTGARSDNSGPWMWCDPEMGHKVSPLT
RCRALVKLECVGNRVPEDVLRDCCQEVANISNEWCRCGDLGSMLRSVYAALG
VGGGPEEVFPGCQKDVMKLLVAGVPALCNVPIPNEAAGTRGVCYWSASTDT
> A0A287JQN1 - Predicted Trypsin/amylase inhibitor
(SEQ ID NO: 90):
MASSQLVPWALLLAVLTTVVATAERDYGEYCRVGKSIPINPLPACREYITRRCA
VGDQQVPDVLKQQCCRELSDLPESCRCDALSILVNGVITEDGSRVGRMEAVPG
CDRERIHSMGSYLTAYSECNLHNPGTPGGDCVLFGGGIS
>P11643- Alpha-amylase/trypsin inhibitor CMd (SEQ ID NO: 91):
MACKSSRSLLLLATVMVSVFAAAAAAAAATDCSPGVAFPTNLLGHCRDYVLQ
QTCAVFTPGSKLPEWMTSAELNYPGQPYLAKLYCCQELAEIPQQCRCEALRYF
MALPVPSQPVDPSTGNVGQSGLMDLPGCPREMQRDFVRLLVAPGQCNLATIH
NVRYCPAVEQPLWI
> A0A287W0A8 - Alpha-amylase/trypsin inhibitor CMa
(SEQ ID NO: 92):
MASKSSITPLLLAAVLASVFAAAAATGQYCYAGMGLPSNPLEGCREYVAQQT
CGVSIAGSPVSTEPGDTPKDRCCQELDEAPQHCRCEAVRYFIGRRSHPDWSVLK
DLPGCPKEPQRDFAKVLVTPGQCNVLTVHNAPYCLGLDI
>P32936- Alpha-amylase/trypsin inhibitor CMb (SEQ ID NO: 93):
MASKSSCDLLLAAVLVSIFAAVAAVGSEDCTPWTATPITPLPSCRDYVEQQAC
RIETPGPPYLAKQQCCGELANIPQQCRCQALRFFMGRKSRPDQSGLMELPGCPR
EVQMDFVRILVTPGFCNLTTVHNTPYCLAMDEWQWNRQFCSS
>P34951- Trypsin inhibitor CMc (SEQ ID NO: 94):
MASCSQHLLSAVAIFSVLAGVATATSIYTCYEGMGLPVNPLQGCRFYVASQTC
GAVPLLPIEVMKDWCCRELAGISSNCRCEGLRVFIDRAFPPSQSQGAPPQLPPLA
TECPAEVKRDFARTLALPGQCNLPAIHGGAYCVFP
>F2DNP3- Thaumatin-like protein TLP8 (SEQ ID NO: 95):
MASLPTSSVLLPILLLVLVAATADAATFTVINKCQYTVWAAAVPAGGGQKLD
AGQTWSINVPAGTTSGRVWARTGCSFDGAGNGRCQTGDCGGKLRCTQYGQA
PNTLAEFGLNKYMGQDFFDISLIDGYNVPMSFVPAPGSTGCPKGGPRCPKVITP
ACPNELRAAGGCNNACTVFKEDRYCCTGSAANSCGPTDYSRF
>F2E994-Predicted ATI-like protein (SEQ ID NO: 96):
MEHFCFLVILSLSGLAMALQLTTPCNAAQAQPQPIYDTDGHELTGNNLYNIMP
VGRNLSDQCVSPSSFREDQCRVRAILTPCKQFRGTDGYVRIRLAEVSDSSDTEA
LPRLSTDVVIDFRGVVTSCVYPLQWYVRQENTKQMHVTAGLLTGMDGCKTPA
GTCLAEIFMFRVEKHGAGYKLMWCPDAPCRDLVLLDYAGRRYLTVEKDGREP
LVVVFKKFHRVTLPPASHPQLG
> F2E8J4 - Bifunctional alpha-amylase/subtilisin inhibitor
(SEQ ID NO: 97):
MGSRRAGLLLLSLILASTALSRSADPPPVHDTDGHELRADANYYVLSANRAHG
GGLTMAPGHGRHCPLFVSQDPNGQHDGFPVRITPYGVAPSDKIIRLSTDVRISFR
AYTTCLQSTEWHIDSELAAGRRHVITGPVKDPSPSGRENAFRIEKYSGAEVHEY
KLMSCGDWCQDLGVFRDLKGGAWFLGATEPYHVVVFKKAPP

### CM Proteins

The levels of three CM proteins, CMa (A0A287W0A8), CMd (P11643) and CMe (P01086) were decreased in the triple null lines (BCD-null) relative to the control cv Sloop with values of ˜75% (p<0.15), ˜66% (p<0.14), and 1% (p<0.001) respectively. In contrast CMc (P34951) was noted to increase in abundance to 194% and the change was not significant in comparison to WT-g (p<0.32) (FIG. 6a-e). CMe was at or below the limit of detection for the C-null, BC-null [CD-null] and the BCD-null line. Surprisingly the CMa was reduced in the triple null but not the other barley genotypes relative to the wild-type control. CMb remained unchanged in the triple null BCD line although was increased in each of the single null lines compared to the wild-type control. The relative amount of all peptides detected for each of the 12 proteins are given in Table 4.

TABLE 4
Summed peak area for all peptides from barley ATIs monitored in the present study.

| Peptide | WT-g | B-null | C-null | D-null | BC-null | BD-null | CD-null | BCD-null |
|---|---|---|---|---|---|---|---|---|
| F2DNP3-AAGGCNNACTVFK | 29329 | 13232 | 16782 | 38612 | 18758 | 14551 | 11689 | 16696 |
| F2DNP3-CTQYGQAPNTLAEFGLNK | 261430 | 842560 | 4627400 | 188884 | 4987800 | 1090950 | 5622300 | 7186000 |
| F2DNP3-CQYTVWAAAVPAGGGQK | 4879 | 5426 | 6651 | 4393 | 5114 | 4471 | 10086 | 7400 |
| F2DNP3-LDAGQTWSINVPAGTTSGR | 1164520 | 5003200 | 25716200 | 899080 | 25144200 | 6336500 | 29721100 | 41262300 |
| F2DNP3-TGCSFDGAGNGR | 1534060 | 4809400 | 20410000 | 1654550 | 22582500 | 5689000 | 24285500 | 37348700 |
| F2DNP3-VITPACPNELR | 2860160 | 8936100 | 31197400 | 3098320 | 32705600 | 11323100 | 34148600 | 50384000 |
| F2E994-DLVLLDYAGR | 258939 | 354810 | 808580 | 300024 | 902720 | 782680 | 732470 | 494960 |
| F2E994-EPLVVVFK | 22616 | 34415 | 33851 | 28778 | 47153 | 36624 | 70912 | 39520 |
| F2E994-LSTDVVIDFR | 4963900 | 5923900 | 10011200 | 7727200 | 10225700 | 8421900 | 10155800 | 8714300 |
| M0UYA9-SQCAGGQVVESIQK | 14745300 | 16704800 | 4426100 | 15503500 | 3164690 | 15199700 | 3862570 | 3038810 |
| M0UYA9-ATVAEVFPGCR | 61799000 | 42793300 | 13896300 | 56019000 | 11130500 | 45801000 | 12993400 | 8829900 |
| M0UYA9-ELGVALADDK | 45380000 | 45572000 | 15963600 | 50861000 | 12915800 | 44874000 | 14209200 | 10418500 |
| M0UYA9-QIAAIGDEWCICGALGSMR | 2382790 | 3445730 | 1053350 | 3435760 | 797440 | 2982340 | 1000700 | 935880 |
| A0A287JQN1-CAVGDQQVPDVLK | 6362200 | 14873400 | 5754700 | 11219300 | 3892540 | 14882800 | 4819200 | 3919780 |
| A0A287JQN1-CDALSILVNGVITEDGSR | 2299570 | 4933700 | 1652070 | 4062500 | 1184660 | 5082500 | 1351600 | 1328860 |
| A0A287JQN1-DYGEYCR | 57303000 | 74405000 | 20824900 | 90290000 | 12911200 | 75041000 | 16164100 | 11778200 |
| A0A287JQN1-ELSDLPESCR | 44276000 | 71888000 | 24460300 | 77578000 | 17766000 | 75849000 | 20183300 | 16353500 |
| A0A287JQN1-SIPINPLPACR | 120240000 | 160759000 | 50133000 | 172273000 | 31971700 | 166838000 | 37997800 | 30787200 |
| A0A287JQN1-MEAVPGCDR | 42216400 | 56057000 | 25368070 | 71449600 | 16905490 | 60296820 | 21465990 | 15316690 |
| A0A287W0A8-CCQELDEAPQHCR | 9764700 | 15070700 | 14456800 | 18459000 | 13558700 | 17110300 | 13245500 | 11382700 |
| A0A287W0A8-EYVAQQTCGVSIAGSPVSTEPGDTPK | 2891390 | 2613450 | 2977440 | 5155100 | 3069830 | 4173500 | 2882990 | 1983070 |
| A0A287W0A8-SHPDWSVLK | 125843000 | 172584000 | 141636000 | 180659000 | 125272000 | 172709000 | 114112000 | 102028000 |
| P01086-CCDELSAIPAYCR | 27346300 | 58479000 | 1219480 | 27709200 | 847380 | 56677000 | 1013660 | 804100 |
| P01086-DSPNCPR | 25623700 | 27563900 | 681180 | 21850700 | 376564 | 26580100 | 443570 | 335145 |
| P01086-LLTSDMK | 97659000 | 100030000 | 1184490 | 143423000 | 652580 | 80015000 | 818280 | 569920 |
| P01086-TYVVSQICHQGPR | 580350000 | 554640000 | 9675200 | 715900000 | 5059800 | 495580000 | 6460300 | 3982930 |
| P11643-DYVLQQTCAVFTPGSK | 25223800 | 31398400 | 27346000 | 34244600 | 21455900 | 35030400 | 21544700 | 18591800 |
| P11643-LLVAPGQCNLATIHNVR | 405090000 | 453690000 | 404127000 | 532670000 | 328215000 | 548920000 | 354828000 | 235574000 |
| P11643-LYCCQELAEIPQQCR | 51612000 | 73911000 | 75061000 | 78663000 | 60873000 | 89008000 | 65720000 | 58665000 |
| P13691-DCCQEVANISNEWCR | 5916500 | 8596300 | 2829250 | 10260800 | 10841600 | 9435800 | 9802600 | 1871870 |
| P13691-LECVGNR | 29547600 | 21847800 | 8063300 | 38973100 | 29519200 | 24366800 | 24189300 | 3957850 |
| P13691-LLVAGVPALCNVPIPNEAAGTR | 12728700 | 15279100 | 3795060 | 17090500 | 12204300 | 15000300 | 10571700 | 1911100 |
| P13691-SVYAALGVGGGPEEVFPGCQK | 11840700 | 16809100 | 4083600 | 17756800 | 13771000 | 15744700 | 12409800 | 2008320 |
| P13691-VPEDVLR | 115633000 | 95901000 | 33501400 | 169210000 | 116987000 | 101383000 | 101053000 | 15544800 |
| P13691-CGDLGSMLR | 91533750 | 96540430 | 27261070 | 140891900 | 88059400 | 84659240 | 81650050 | 14829158 |
| P32936-DYVEQQACR | 74373000 | 129612000 | 91847000 | 95522000 | 77247000 | 125959000 | 77818000 | 72359000 |
| P32936-EVQMDFVR | 174630000 | 304338000 | 230991000 | 230427000 | 185111000 | 268971000 | 191326000 | 181834000 |
| P32936-IETPGPPYLAK | 165613000 | 288792000 | 207509000 | 188931000 | 170329000 | 286866000 | 175927000 | 159570000 |
| P32936-QQCCGELANIPQQCR | 58661800 | 90335800 | 68125200 | 66949000 | 53394900 | 81775400 | 55505700 | 49211300 |
| P32936-SRPDQSGLMELPGCPR | 89397900 | 154751700 | 128648400 | 106306700 | 103098400 | 148367100 | 111536700 | 107983600 |
| P34951-AFPPSQSQGAPPQLPPLATECPAEVK | 5609400 | 0 | 0 | 3696800 | 0 | 7866300 | 17818500 | 7883700 |
| P34951-ELAGISSNCR | 257849000 | 461470000 | 474310000 | 397944000 | 414446000 | 529980000 | 383887000 | 364156000 |
| P34951-FYVASQTCGAVPLLPIEVMK | 18129100 | 40531900 | 56565000 | 29619700 | 46472000 | 41894000 | 43971000 | 44594900 |
| F2E8J4-SADPPPVHDTDGHELR | 66524 | 48696 | 140766 | 71460 | 97775 | 73294 | 155027 | 103078 |
| F2E8J4-ADANYYVLSANR | 5293800 | 2522990 | 8203600 | 3992540 | 4614800 | 2842200 | 7361200 | 4739000 |
| F2E8J4-HCPLFVSQDPNGQHDGFPVR | 1178830 | 1656340 | 1932470 | 1300080 | 2614340 | 2462590 | 2201730 | 1841500 |
| F2E8J4-ITPYGVAPSDK | 2127900 | 1973450 | 2791770 | 1912060 | 1779060 | 1904090 | 2249000 | 1592750 |
| F2E8J4-AYTTCLQSTEWHIDSELAAGR | 373785 | 676950 | 2026170 | 290836 | 1125550 | 586140 | 1923150 | 1417500 |
| F2E8J4-HVITGPVK | 485460 | 540570 | 1639430 | 247106 | 981900 | 537980 | 1632430 | 1126480 |
| F2E8J4-YSGAEVHEYK | 660140 | 913620 | 2400860 | 634520 | 1606330 | 822440 | 2324870 | 2008950 |
| F2E8J4-GGAWFLGATEPYHVVVFK | 1229860 | 1005250 | 2544870 | 951070 | 1361820 | 1157630 | 2185880 | 1463210 |
| F2EJF0-QILEHQLTGR | 2365370 | 5474240 | 127962 | 3163150 | 95349 | 5343750 | 160412 | 161049 |
| F2EJF0-DYEQSMPPLR | 2817937 | 6372430 | 104995 | 3624537 | 77515 | 6846940 | 93072 | 106564 |
| F2EJF0-QQQGEGFSGEGAQQKPK | 1075742 | 2896582 | 26556 | 341739 | 21202 | 1632142 | 42192 | 45820 |

CMa to CMe proteins are encoded by a disperse multigene family. For instance, genes for CMa, CMb, CMc, CMd and CMe in barley are located on chromosomes 1, 4, 3, 4 and 3 respectively (Lazaro et al., 1985). CMa, CMb and CMd inhibitors of alpha-amylase, whilst CMc and CMe are reported to inhibit trypsin (Barber et al., 1989; Barber et al., 1986). Barber et al. (1986) reported that CMb and CMd did not show any inhibitory activity. The alpha-amylase inhibitor CMa (FIG. 6a) and trypsin inhibitor CMc (FIG. 6c) genes are located on chromosome 1, and these two proteins were found to be decreased ˜75% and increased ˜194%, respectively in the triple null lines. The divergence between CMa and CMb on the one hand and CMe with respect to trypsin inhibition ability may involve the regulatory properties of these genes and their locations (Barber et al., 1986), as the gene expression patterns are differentially correlated with the different high-lysine mutations which were observed in barley cultivars Risø 1508, Risø 527, Risø 7 and Risø 56 (Lazaro et al., 1985). Notably, the CMe was down-regulated ˜100 fold in comparison to WTs and for the first time it has been demonstrated that this ATI class has been downregulated in the BCD-null lines which may be resulted from the synergistic effect resulted from cross-breeding between single and double-null lines.

The alpha-amylase inhibitor CMa (FIG. 6a) and trypsin inhibitor CMc (FIG. 6c) genes are located on chromosome 1. It was seen here that CMc was increased (˜194%; p<0.32) in the triple null lines, while CMa and CMe was decreased (˜75 and ˜100-fold respectively) in the triple null lines (FIG. 6a, c and e). To further annotate these peptides from trypsin inhibitors, a BLAST search was performed. Our BLASTp search revealed that peptides from trypsin inhibitors (CMc and CMe) are unique to these two proteins from H. vulgare. CMe is a well-known trypsin inhibitor (Lazaro et al., 1985) and found to be three times more potent than CMc (Barber et al., 1986). The genes for two closely related CM proteins, i.e. CMb and CMd, are located on chromosome 4. In the present study, two proteins, CMb (˜100%, p<0.90) and CMd (˜66%, p<0.14), were found to be decreased in the triple null line. Studies have shown minimal trypsin inhibitory activity in vitro when they were tested independently (Barber et al., 1986). Interestingly, another study reported that the barley tetrameric inhibitor subunits, including CMa, CMb and CMd, are active against trypsin and alpha-amylase from the yellow mealworm beetle (Tenebrio molitor) when they are associated together (Sanchez-Monge et al., 1986). Interestingly the presence of all three hordein mutations to create the BCD-null line had a synergistic effect on reducing accumulation of the CMa and CMd (A-hordeins) as defined by the peptides above.

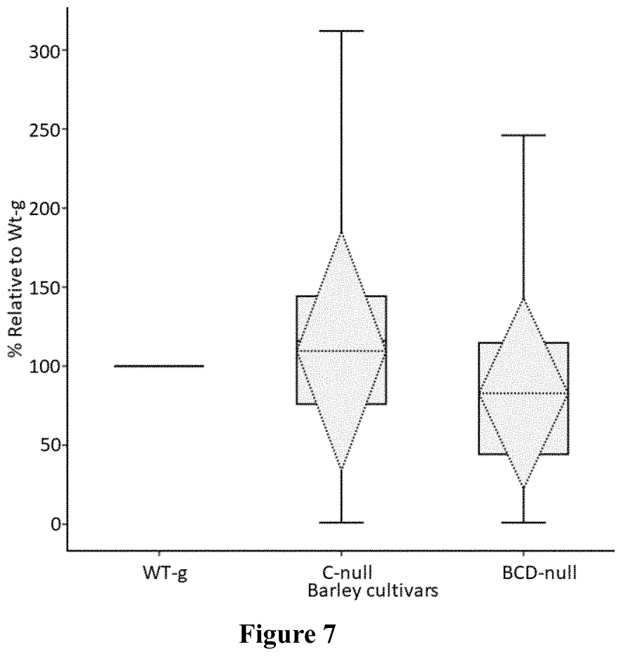

To identify the overall peptide changes across ATI CMa-e proteins affect the WT-g and C- and BCD-null lines, all peptides from these proteins were summed and the data was converted to a percentage relative to wild-type for ease of data comparison. Result shows that the overall CMa-e peptides were upregulated in WT-g lines (calculated mean value for all monitored peptides from WT-g lines: 545) whilst the peptides were down-regulated ˜2-fold (calculated mean value for all monitored peptides from BCD-null lines: 301) (FIG. 7). Notably, peptides from CMa-e proteins were also down-regulated in the C-null lines ˜2-fold (calculated mean value for all monitored peptides from BCD-null lines: 353). Surprisingly, a synergistic effect was seen to result in an overall reduction in CMa-e proteins in the triple null line, relative to the C-null line, suggesting it is a cereal suitable for reducing or preventing a gastrointestinal inflammatory response such as in NCGS from allergen exposure.

### Other Predicted ATI Proteins

In addition to the CM proteins, a significantly decreased (3-fold; p<0.05) uncharacterized (A0A287JQN1) protein in the triple null line was identified (FIG. 60. Gene ontology and molecular function analysis revealed that this protein has serine-endopeptidase inhibitor activity and contains a conserved domain for trypsin-alpha amylase inhibitor (AAI). Notably, this type of protein can stop, prevent or reduce serine-endopeptidases, enzymes that catalyse the hydrolysis of non-terminal peptide bonds in a polypeptide chain; a serine residue (and a histidine residue) are at the active centre of the enzyme. Sequence alignment on UniProt database showed that is the most closely matched (high conservation of cysteine; identity match 53.5%) to a protein from Brachypodium distachyon (UniProt ID: I1H2M2; uncharacterized protein with AAI domain).

Thaumatin is postulated to increase in abundance to compensate for the decreased level of hordeins and ATIs in the ULG lines and may represent an important nutrient reserve for plant growth and human nutrition (Shewry, 1999). Thaumatin-like proteins (TLPs) are classified as the pathogenesis-related family 5 (PR-5) and are expressed in plants in response to pathogen attack, abiotic stress and developmental signals. In the food industry, thaumatin has been used as a sweetener and flavor modifier (Tschannen et al., 2018). Two selected peptides from thaumatin were increased 20-fold in the triple null line (FIG. 8a). Thaumatin remained unchanged in WT irrespective of growing conditions, but was noted to increase across the single, double and triple null lines. Notably, the thaumatin expression between WT- and D-null line was similar (p<0.07) as the D-hordein line is represented by an alteration to a single gene that constitutes ˜1-2% of the total hordein content.

Globulins are abundant seed storage proteins that account for up to 50% of the total protein in the mature cereal grains. Here, a predicted protein (F2EJF0) sharing 98% homology with proteins annotated as globulins from Hordeum vulgare was quantified. This protein was significantly reduced (˜100-fold, p<0.001) in the triple null line (FIG. 8b). Unlike other globulins, such as Q84NG7 that was found to be increased in the triple null line possibly in compensation for the loss of the hordeins, F2EJF0 (98.7% homology to globulin) was substantially decreased in the triple null line (p<0.001). Both Q84NG7 and F2EJF0 were identified as globulin-like proteins (total number of amino acids 224 with a conserved domain extending from 45-214) and they have shared common conserved domains. For example, Q84NG7 (globulin) and F2EJF0 (predicted protein/globulin) both proteins shared conserved domains such as “allergen/soft/tryp_amyl_inhib” and “trypsin-alpha amylase inhibitor domain family”.

Molecular function analysis (using Gene Ontology and annotation database on QuickGO server) of F2EJF0 protein revealed that this protein can interact selectively and non-selectively with an immunoglobulin of the IgE isotype and initiates the positive regulation of type I hypersensitivity in humans. The Bifun_inhib/LTP/seed_sf (InterPro: IPR036312) domain is one of the member of ‘Bifunctional inhibitor/plant lipid transfer protein/seed storage helical domains family’. This domain is closely related to allergen-like proteins (http://www.ebi.ac.uk/interpro/entry/IPR000528) and has a molecular function known as ‘lipid binding’ which helps to interact proteins selectively or non-selectively with a lipid in order to initiate an allergenic reaction. It should be noted that many proteins in grain protein databases have names that do not reflect their function.

BMAI-1 (MOUYA9) and BDAI-1 (P13691) (FIG. 8c-d, Table 4) had decreased levels (6-fold) in the triple null line. BMAI-1 was seen to decrease in abundance across the double null lines compared to the single null lines and wildtype cv Sloop (WTg). BDAI-1 (FIG. 8c) is notably increased in abundance in the BD- and CD-double-null lines compared to the single nulls and wild-type. The BCD triple null line was decreased 6-fold compared to the cv Sloop (WTg). Molecular function analysis of BDAI-1 revealed that this protein inhibits insect type alpha-amylase and serine-type endopeptidases and also part of the allergome database (allergome code: 8778). In an in vitro study, the orthologous BDAI-1 protein from T. aestivum was shown to target both mammalian trypsin and alpha-amylase with independent binding sites and moderately high affinity (Cuccioloni et al., 2016). An earlier study found that the orthologous protein of BDAI-1 from T. aestivum was more active against human salivary alpha-amylase than against an insect enzyme (Garcia-Olmedo et al., 1991). A reduction in both BMAI-1 and BDAI-1 would be favourable for reducing allergenic reaction to barley. Interestingly the triple null line showed a synergistic reduction in these proteins.

The other proteins investigated were alpha-amylase/subtilisin inhibitor-like protein (F2E994) and bifunctional alpha-amylase/subtilisin inhibitor-like protein (F2E8J4), which are also known for their role in plant defence. Here, peptides representing these two proteins were quantified revealing an increase in abundance to levels ˜183% and ˜230% relative to the WT barley respectively (FIG. 8e, f and Table 4).

### Example 8. Characterization of ATI Orthologues in Barley and Wheat

Inhibitory proteins from grains can act directly by targeting specific pro-inflammatory receptors, and indirectly by reducing the activity of digestive enzymes, which causes the accumulation of undigested peptides with potential immunogenic properties. To date, only wheat-derived ATIs have been extracted and fractionated with a goal of elucidating the inflammatory pathways of NCGS while other grains were used whole (Zevallos et al., 2017). To identify barley ATI orthologues in the wheat proteome, BLAST searches were performed against the UniProt database. The result show that the identified barley proteins belong to the protease inhibitor 16 (cereal trypsin/alpha-amylase inhibitor) family, which comprises 27 entries from barley, wheat, rice and maize. Whether the identified proteins or their orthologues had been reported in previous animal model-based experiments was then explored. Eight proteins mapping to barley ATIs and 9 belonging to wheat ATI families were identified. Next, a phylogenetic tree (FIG. 5a) and multiple sequence alignment (FIG. 5b) was used to identify orthologous proteins from barley and wheat varieties. In addition, a comprehensive literature search was performed for ATIs that have been implicated in NCGS and by homology searching have identified the orthologous barley ATI protein accession from the Uniprot database (Table 5). The discovery data was then searched for evidence of these potentially toxic barley ATI components and when found the reported level in the ULG line relative to WT cv Sloop barley is reported in Table 5. Only one study on the human allergenicity effects of ATIs was conducted (Armentia et al., 1993), however, no animal studies have been conducted to elucidate their role in NCGS. The two wheat ATIs that have been strongly associated with NCGS are 0.19 (wheat, P01085) and CM3 (wheat, P17314).

TABLE 5
List of barley ATIs detected in this study, their detection level and
related orthologous wheat ATIs that have been implicated in NCGS

| | | Relative | | | |
|---|---|---|---|---|---|
| | | amount in | Relative | | |
| Barley | | triple null | amount | Wheat | |
| Uniprot | | (BCD-null) line | in C-null | Uniprot | Homology |
| accession | Name | (cf WTf) ^{a} | (cf WT) ^{a} | accession ^{b} | % |
| P13691 | alpha-amylase | ~16% | ~30% | P01083 | 62.1 |
| | inhibitor BDAI-1 | | | | |
| | (wheat ATI 0.28) | | | | |
| C3VX00 ^{a} | Dimeric alpha-amylase | ND | ND | P01085 | 100.0 |
| | inhibitor | | | | |
| | (wheat ATI 0.19) | | | | |
| | (wheat ATI 0.53) | ND | ND | P01084 | 94.4 |
| P28041 ^{a} | alpha-amylase/trypsin | ND | ND | P16850 | 84.1 |
| | inhibitor CM1 | | | | |
| P01086 | Trypsin inhibitor CMe | ~1% | ~1% | N/A | N/A |
| A0A287W0A8 | alpha-amylase/trypsin | ~75% | ~107% | P16851 | 85.5 |
| | inhibitor CMa | | | | |
| | (wheat ATI CM2) | | | | |
| P11643 | alpha-amylase/trypsin | ~66% | ~103% | P17314 | 83.0 |
| | inhibitor CMd | | | | |
| | (wheat ATI CM3) | | | | |
| P32936 | alpha-amylase/trypsin | ~100% | ~128% | P16159 | 90.2 |
| | inhibitor CMb | | | | |
| | (wheat ATI CM16) | | | | |
| | (wheat ATI CM17) | | | P16852 | 73.1 |
| M0UYA9/P16968 | alpha-amylase | ~17% | ~26% | N/A | N/A |
| | inhibitor BMAI-1 | | | | |
| A0A287JQN1 | Uncharacterised protein | ~31% | ~48% | N/A | N/A |
| | (similar to Baker's | | | | |
| | asthma allergen) | | | | |

^{a} ND, not detected in WT barley and hence not measured by LC-MRM-MS.
^{b} N/A, no wheat orthologue in public database.

Interestingly, the triple null and C-null lines had reduced overall ATI abundance as summarised in Table 5 compared to the wild-type Sloop control. Even more so the triple null lines although showing no significant negative phenotypes with respect to the grain or growth performance had even further reduced ATI abundance.

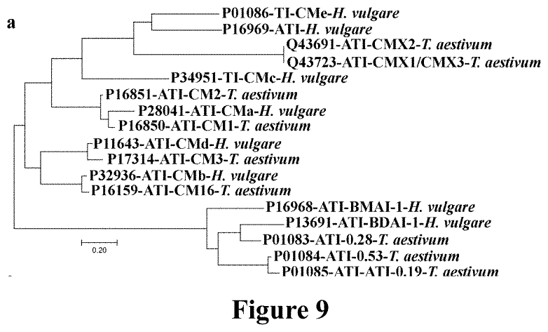

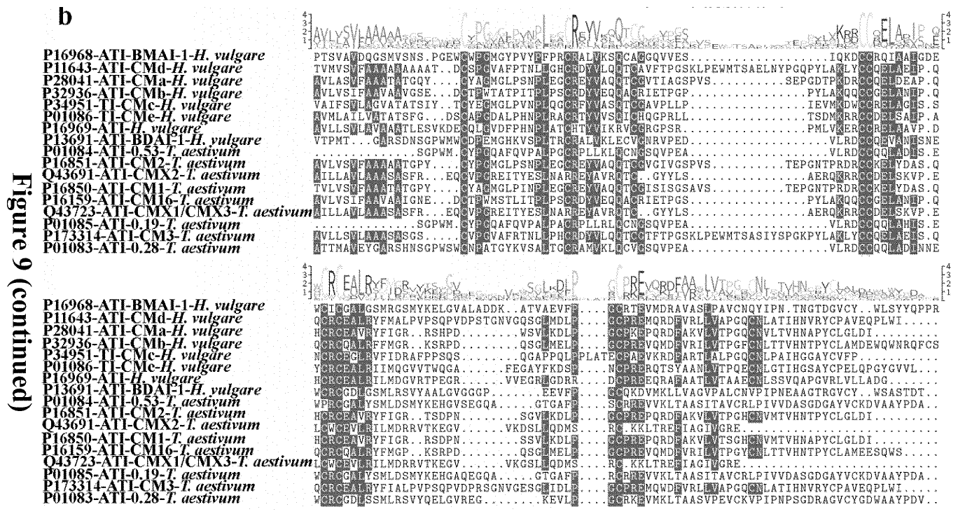

Barley and wheat ATIs have conserved disulfide linkages through the conserved cysteine residues. The results showed that two proteins from barley, BMAI-1 (MOUYA9, ˜6-fold reduction in triple null line) and BDAI-1 (P13691, 6-fold reduction in triple null line), were closely matched to wheat ATI species 0.28 and 0.19, respectively (FIG. 9a). This assumption, i.e. wheat 0.19 and 0.28 is closely matched with barley BDAI-1 and BMAI-1 was made based on the results from phylogenetic analysis (FIG. 9a). Although the sequence homology between P13691 (BDAI-1) and P01083 (wheat ATI 0.28) was ˜62% by BLASTp (59% similarity by clustal omega alignment), the homology score between P13691 (BDAI-1) and P01085 (wheat ATI 0.19) was lower at 49% by BLASTp (˜35% by clustal omega alignment). One explanation for this low homology may be due to incomplete sequence available for wheat ATIs in UniProt database. The main difference is coming due to the missing of signal peptide in the wheat ATI 0.19 (P01085). To test whether removing the missing signal peptide for P13691 had any influence on sequence homology, another alignment was performed between P13691 and P01085 (wheat ATI 0.19). The results showed that removing the signal peptide improved the similarity to ˜43%, but P13691 (BDAI-1) shares greater similarity with P01083 (wheat ATI 0.28).

Likewise, barley MOUYA9 (BMAI-1) shows closer correlation with P01083 (wheat ATI 0.28) at ˜50% (˜43% by clustal omega alignment), whereas correlation with P01085 (wheat ATI 0.19) was lower at ˜44% (˜31% by clustal omega alignment). Removing the signal peptide from the wheat ATI 0.19 (P01085) only increased the similarity slightly (˜38% by clustal omega alignment).

The wheat ATI 0.19 proteins have been shown to be the most prevalent activators of TLR4 (100-fold higher biological activity than non-gluten grains) in the NCGS disease pathway and were highly resistant to intestinal proteolysis (Zevallos et al., 2017). Additionally, the trypsin inhibitor CMe (P01086) was down-regulated up to ˜100-fold in the double- and triple-null lines. Homology searching revealed that CMe is orthologous to CMX1-3 proteins from T. aestivum that were found to activate innate inflammatory pathways in an NCGS murine model (Zevallos et al., 2017).

### Example 9. Generation of Hull-Less Barley Mutants

The barley grains for selections ULG3.0, ULG3.1 and ULG3.2 were all hulled, which is of benefit to the brewing industry as the spent husks form a filtration bed during the final stages of wort filtration (lautering). However, barley grain hulls have large numbers of tiny silica spikes and therefore the hulls need to be removed by pearling before human consumption. An alternative approach is to produce hull-less grains by genetic means. Therefore, plants of ULG3.0 were crossed with a hull-less barley variety designated Barleymax II (WO2011/011833) and a hull-less, hordein B-, C-, D-triple null mutant (Hor2-lys3a-Hor3) plant selected which was wild-type for the SSHa gene.

Some F6 hordein triple-null hull-less selections (eg A7_1) contained less than 0.1 ppm total hordein in flour after three rounds of single seed descent.

### Example 10. Mutagenesis of Barley

To isolate mutants in selected genes, e.g., ATI genes, in barley, ethylmethanesulfonate (EMS) mutagenesis is carried out as described by Caldwell (2004). Approximately 45,000 (1.5 kg) grain of ULG3.0 is mutagenised as follows: the grain is imbibed in 2.5 L of distilled water for 4 hr at room temperature with aeration. The water is changed every hour for the duration of imbibition. The seeds are then incubated in 2.5 L of freshly prepared 30 mM EMS in 0.1 M phosphate buffer (pH7) for 16 h at room temperature with aeration. The seeds are then washed with 2.5 L of 100 mM sodium thiosulphate for 10 min at room temperature. The washing with thiosulphate is repeated, and the grains then rinsed thoroughly with 2×2 L of distilled water for 30 min at room temperature with aeration. The seeds are air-dried overnight on absorbent filter paper under a flowing air stream, prior to planting in the field the next day. Bulk M2 seeds are harvested, pooled and analysed for mutants. The mutational frequency after such treatment is typically approximately 1 mutant in a gene of interest per 1000 seeds. The seeds are then screened for the loss of expression of the gene of interest by dot blot on half-grains using a monoclonal antibody against the protein encoded by the gene of interest. Alternatively or additionally, levels of ATI proteins can be measured as described in Examples 5 to 8. The lack of expression of the gene of interest in these grains can also be confirmed by Western blotting using the monoclonal antibody, and by mass spectrometry for a specific peptide in the protein.

### Example 11. Production of Beer from Barley Grain Having a Reduced Level of ATIs

Brewing trials were carried out using malt produced from ULG2.0 grain by standard methods, and compared with the control wild-type barley cv. Gairdner. Gairdner is a high yielding mid to late maturing semi-dwarf 2-row barley variety grown widely throughout the Australian cereal growing regions. It produces good grain size under favourable conditions, producing moderate extract levels, fermentability and diastatic power, and therefore represents an industry “standard” so is ideal for its use as a control in brewing evaluation trials. The malt produced from ULG2.0 had a slightly higher moisture content of 5.7% compared to Gairdner malt (5.0%) and in appearance was significantly different to Gairdner malt, in that the grain appeared dented and shriveled. The Diastatic Power (DP) of the ULG2.0 malt was 54 WK, much lower than the DP for Gairdner at 299 WK. Malted barley generally has a DP of at least 250 KW. However, the ULG2.0 malt was negative for starch after 20 min mash time and achieved an LG result of 1.7° Plato.

The malt was milled by two passes through a 2-roller mill and achieved satisfactory cracking of the grain. The malt would be best suited for more complex milling in a six roller mill due to its grain morphology. Alternatively, a hammer mill in conjunction with a mash filter could be used, rather than a lauter tun for wort separation. The milled product was mashed by standard methods, at an initial temperature of 65° C. for 20 min, then 74° C. for 5 min, with the addition of extra sparge liquor. Overall, the shriveled morphology of the ULG2.0 malt made it difficult to mill and mash satisfactorily in comparison to regular malt. The mashed products were then lautered, where again the shriveled morphology of ULG2.0 caused difficulties. The lautering bed fell apart with chanelling so the run off needed to be stopped and the lautering bed re-raked. A considerable amount of potential extract was lost during the lautering process due to the inefficient milling, achieving an all in kettle value of 10.96° and 11.12° Plato when the target was to achieve 14° Plato. The pH, EBC colour and beta-glucan levels were acceptable. The milling deficiency also meant there was not an efficient formation of a bed of husks to act as a filter medium, contributing to a lower the expected extract. The clarity of run-off was initially very hazy, due to poor bed formation, although the wort clarity improved to be acceptable by 30 L of runoff.

The resultant wort was fermented with yeast strain Saccharomyces uvarum A at 18.5° C. for 120 hr. The fermentation profile was normal, there was no extended lag phase at the start of fermentation, although significant levels of diacetyl remained even after an extended diacetyl rest phase was given after the end of fermentation gravity was reached. Diacetyl rest is where the beer is left at higher fermentation temperatures prior to chilling the beer to 0° C. to allow the yeast to reabsorb and metabolise diacetyl. Generally 24 hr at end of fermentation is required to allow the yeast time to break this product down. This did not occur with both of the ULG2.0 trial brews. Isomerised hop extract was added at 30 mg/L and the liquor clarified with addition of Silica Hydrogel. The finished bright beer had a lower physical stability than the control brew. Initial chill haze was considered high and the forced chill haze results were outside of normal specifications. However, the chill haze did not form particulates.

The finished beer was subjected to sensory analysis by a panel of trained brewers, and despite the difficulties in the milling and lautering processes, the results were surprisingly good given the issues associated with the brewing performance of the malt. DMS (dimethyl sulphide) was the predominate flavour; however this was not seen as objectionable. The presence of DMS is often considered a flavour fault in Australian beer but is generally well accepted in European, particularly German beers. The brewers' comments were that the ULG2.0 beers were not too dissimilar to the control beers and that they were very passable as a beer, and reminiscent of German Beers. There was no overt “grainy” or “cereal” type flavours and no harshness or astringency with the ULG2.0 beer as can be typical of commercial beers marketed as “Gluten free”. Overall the flavour profile was acceptable and reasonable.

Beer was made from barley grain of ULG3.0 by the same method as for ULG2.0, and is made from barley grain of ULG3.2. Malting of the ULG3.0 grain was improved relative to the ULG3.0 grain, mainly because the milling step was improved due to the grain being less shriveled. The beer made from ULG3.0 was of a good quality with an acceptable and reasonable flavor. The improved grain morphology (size and shape) of the grain from ULG3.2 provides for easier milling and lautering in the brewing process, providing beer with less than 1 ppm of total hordeins.

### Example 12. Characterization of ATIs in Beer by MRM Mass Spectrometry

In order to characterize peptide fragments, the wort and beer were passed through a 10 kDa molecular weight cut-off filter and analyzed without enzymatic digestion. 1D-PAGE analysis revealed that the filtration step was efficient in removing proteins from beer. The inventors have found that ATIs persist in beers. Three beers were tested: (1) beer produced with wildtype barley and brewed using the enzyme prolyl endoprotease (PEP) that is present in Brewer's Clarex, a haze-removing enzyme preparation; (2) beer produced using BCD null barley as described in (WO 2014/197943); and (3) a commercial beer Pionier™ made by German brewer, Radeberger Gruppe KG using BCD null barley (Kebari™). In this discovery analysis (not quantitative), the inventors detected ATIs such as A0A287W0A8 and P32936 in all three beers. These were ATIs that were not vastly altered in the ULG line. Notably, P01086 was only detected in beer sample 1, not in either of the BCD null barley-brewed beers. Indicating that the protein is also decreased in beers produced from BCD null barley. Likewise, the data shows a decreased score, which estimates a lower level or abundance, for AOA287JQN1 in the BCD-null barley brewed beers (samples 2 and 3) compared to the beer brewed from wild-type barley (sample 1). This protein was decreased to ˜15% relative to WTg in ULG (BCD null barley).

To confirm these results beers made from ULG are analysed with reference to those brewed from cv Sloop and show the levels using LC-MRM-MS as has been presented in Example 3 for the barley flour. Quantifying ATIs in beer would be undertaken by analyzing the ATI content of a selection of beers, including Sloop (a single cultivar beer), R56 (B-null), R1508 (C-null), G* (BC-null) and Pionier a commercial low gluten beer. Duplicate samples from separate bottles would be treated by reduction, alkylation and digestion and then analyzed by MRM mass spectrometry (Example 1). The MRM assay would confirm the relative changes in abundance of the ATIs.

### Example 13. Production of Food Using ULG Barley

Two small-scale (10 g) breads are baked using the ULG3.0 and ULG3.2 barley lines. Small-scale loaves are baked for test purposes, but the method can be readily scaled up to commercial quantities. One bread is made with 100% ULG barley flour as the flour ingredient, milled as described above, while the second bread is made with a blend of 30% flour and 70% commercial non-gluten flour such as rice flour as the flour ingredient. Flour (13.02 g) and the other ingredients are mixed into a dough, to peak dough development time in a 35-g mixograph. The recipe to be used, based on the 13.02 g of flour in each case is: flour 100%, salt 2%, dry yeast 1.5%, vegetable oil 2%, and improver 1.5%. The water addition level is based on the micro Z-arm water absorption values that are adjusted for the full formula. The moulding and panning are done in two-stage proofing steps at 40° C. and 85% room humidity. Baking is done in a Rotel oven for 14 min at 190° C. The ATI levels present in the breads are then measured using, for example, the MRM mass spectrometry methods described in Example 1.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU 2020900312 filed 5 Feb. 2020, the entire contents of which are incorporated herein by reference.

### REFERENCES

Abdullah et al. (1986) Biotechnology 4:1087.
Almeida and Allshire (2005) TRENDS Cell Biol 15: 251-258.
Altenbach et al. (2011) BMC Research Notes 4:242.
Armentia et al. (1993) Clinical & Experimental Allergy 23:410-415. Barber et al. (1986) Biochimica et Biophysica Acta (BBA)-Protein Structure and
Molecular Enzymology 869:115-118.
Barber et al. (1989) FEBS Letters 248:119-122.
Bibikova et al. (2001) Mol. Cell. Biol. 21: 289-287.
Bibikova et al. (2002) Genetics 161:1169-1175.
Bellinghausen et al. (2018) Journal of Allergy and Clinical Immunology 143:201-212.
Bose et al. (2019) Journal of Chromatography A 1600:55-64.
Bourque (1995) Plant Sci. 105: 125-149.
Brennan et al. (1998) J Cereal Sci. 28:291-299.
Caldwell (2004) The Plant Journal 40:143-150.
Capecchi (1980) Cell 22:479-488.
Casella et al. (2016) Minerva Gastroenterologica Dietologica 63:32-37.
Catassi et al. (1994) Lancet 343:200-203.
Catassi et al. (2013) Nutrients 5:3839-3853.
Catassi et al. (2015) Nutrients 7: 4966-4977.
Certo et al. (2012) Nat Methods 8:941-943.
Cheng et al. (1996) Plant Cell Rep. 15:653-657.
Clapp (1993) Clin. Perinatol. 20:155-168.
Colgrave et al. (2012) J. Proteome Res. 11: 386-396.
Colgrave et al. (2015) Journal of Proteome Research 14:2659-2668.
Colgrave et al. (2016) Journal of Proteomics 147:169-176.
Colgrave et al. (2016) Analytical Chemistry 88:9127-9135.
Colgrave et al. (2017) Food Chemistry 234:389-397.
Comai et al. (2004) Plant J 37: 778-786.
Cuccioloni et al. (2016). Food Chemistry 213:571-578.
Curiel et al. (1992) Hum. Gen. Ther. 3:147-154.
Davies et al. (1993) Cell Biology International Reports 17:195-202.
Demirer et al. (2019) Nature protocols 14:2954-2971.
Doll (1983) Barley seed proteins and possibilities for their improvement. In “Seed
Proteins: Biochemistry, Genetics, Nutritional Value”, Gottschalk W, Muller HP (eds). Martinus Nijhoff, The Hague:207-223.
Doll et al. (1973) Barley Genetics Newsletter 3:12-13.
Doyon et al. (2010) Nat. Methods 7:459-460.
Doyon et al. (2011) Nat. Methods 8:74-79.
Dupont et al. (2011) Proteome Science 9:10.
Eglitis et al. (1988) Biotechniques 6:608-614.
Fehr, (1987) In: Breeding Methods for Cultivar Development, Wilcox J. ed., American Society of Agronomy, Madison Wis.
Field et al. (1982) Theoretical and Applied Genetics 62:329-336.
Finnie et al. (2002). Plant Physiology 129: 1308-1319.
Fujimura et al. (1985) Plant Tissue Culture Letters 2:74.
García-Olmedo et al. (1991) In Barley: Genetics, Molecular Biology and Biotechnology. ed PR S. CAB International.
García Olmedo et al. (1992). In Barley: Genetics, Biochemistry, Molecular Biology and Biotechnology. ed Shewry P. R. CAB International: Wallingford U.K., pp 335-350.
Graham et al. (1973) Virology 54:536-539.
Grant et al. (1995) Plant Cell Rep. 15:254-258.
Groenen et al. (1993) Mol. Microbiol. 10:1057-1065.
Guo et al. (2010) J. Mol. Biol. 400:96-107
Haft et al. (2005) Computational Biology 1(6):e60
Haseloff and Gerlach (1988) Nature 334:585-591.
Henikoff et al. (2004) Plant Physiol 135: 630-636.
Hoe et al. (1999) Emerg. Infect. Dis. 5:254-263.
Ishino et al. (1987) J. Bacteriol. 169:5429-5433.
Janssen et al. (2002) OMICS J. Integ. Biol. 6:23-33.
Jaradat (1991) Theor Appl Genet 83:164-168.
Kalunke et al. (2020) Preprints doi:10.20944/preprints202007.0581.v1
Kikuchi et al. (2003). Theor Appl Genetics 108: 73-78.
Kim et al. (1996) Proc. Natl. Acad. Sci. USA 93:1156-1160
Kim et al. (2012) Genome Res. 22:1327-1333.
Klee et al. (1985) In: Plant DNA Infectious Agents, Hohn and Schell, eds., Springer-Verlag, New York, pp. 179-203
Koziel et al. (1996) Plant Mol. Biol. 32:393-405.
Kreis and Shewry (1989) BioEssays 10:201-207.
Kumar et al. (2018) Molecular Biology and Evolution 35:1547-1549.
Lazaro et al. (1985) The FEBS Journal 149:617-623.
Lee et al. (2007). Journal of Human Nutrition and Dietetics 20:423-430.
Lu et al. (1993) J. Exp. Med. 178:2089-2096.
Ludvigsson et al. (2013) Gut 62:43-52.
Makharia et al. (2015) Nutrients 7(12):10417-10426.
Masepohl et al. (1996) Biochim. Biophys. Acta 1307:26-30.
McConnell Smith et al. (2009) PNAS 106:5099-5104.
Millar and Waterhouse (2005) Funct Integr Genomics 5:129-135.
Miller et al. (2007) Nat. Biotechnol. 25:778-785.
Moehs et al. (2019) Plant Physiol. 179:1692-1703
Mojica et al. (1995) Mol. Microbiol. 17:85-93.
Mojica et al. (2000) Mol. Microbiol. 36:244-246.
Mundy et al. (1983) Carlsberg Research Communications 48:81-90.
Nakata et al. (1989) J. Bacteriol. 171:3553-3556.
Nielsen et al. (2004) Biochimica et Biophysica Acta (BBA)-Proteins and Proteomics 1696:157-164.
Orman-Ligeza et al. (2019) bioRxiv doi: https://doi.org/10.1101/2019.12.18.880955
Pasquinelli et al. (2005) Curr Opin Genet Develop 15: 200-205.
Perriman et al. (1992) Gene 113: 157-163.
Ramirez et al. (2012) Nucleic Acids Res. 40:5560-5568.
Ruiz-Medrano et al. (1992) Plant Molecular Biology 20:1199-1202.
Samuel et al. (2002) Journal of Biological Chemistry 277:35267-35273.
Sanchez-Monge et al. (1986) FEBS Letters 207:105-109.
Sanchez-Monge et al. (1989) European Journal of Biochemistry 183:37-40.
Saitou & Nei (1987) Molecular Biology and Evolution 4:406-425.
Sapone et al. (2012) BMC Medicine 10:13.
Senior (1998) Biotech. Genet. Engin. Revs. 15: 79-119.
Shewry (1995) Biological Reviews of the Cambridge Philosophical Society 70:375-426.
Shewry and Halford (2002) Journal of Experimental Botany 53:947-958.
Shewry and Tatham (1990) Biochemical Journal 267:1-12.
Shewry, et al., (1999) The prolamins of the Triticeae. In Seed Proteins, Klewer: London, 1999; pp 35-78.
Shilov et al. (2007) Molecular & Cellular Proteomics 6:1638-1655.
Shippy et al. (1999) Mol. Biotech. 12: 117-129.
Singh & Skerritt (2001) Journal of Cereal Science 33:163-181.
Slade and Knauf (2005) Transgenic Res 14: 109-115.
Smith et al. (2000) Nature 407: 319-320.
Strobl et al. (1998) Structure 6:911-921.
Szczepek et al. (2007) Nat. Biotechnol. 25:786-793.
Tang et al. (2008) Journal of Proteome Research 7:3661-3667.
Tanner et al. (2013) Plos One: 8:e56456.
Tanner et al., (2010). Aliment Pharmacol Ther. 32: 1184-1191.
Toriyama et al. (1986) Theor. Appl. Genet. 205:34.
Tschannen et al. (2017) American Journal of Industrial Medicine 60:664-669.
van Embden et al. (2000) J. Bacteriol. 182:2393-2401.
Wagner et al. (1992) Proc. Natl. Acad. Sci. USA 89:6099-6103.
Walusiak et al. (2004) Allergy 59:442-450.
Wang et al. (2012) Genome Res. 22:1316-1326.
Waterhouse et al. (1998) Proc. Natl. Acad. Sci. USA 95: 13959-13964.
Weissbach et al. (1988) In: Methods for Plant Molecular Biology, Academic Press, San Diego, Calif.
Zevallos et al. (2017) Gastroenterology 152:1100-1113. e1112.

1. A method of producing a food or malt-based beverage ingredient, or a food or a malt-based beverage, for consumption by a subject with a non-coeliac gastrointestinal sensitivity, the method comprising
(i) processing barley grain to produce processed barley grain, malt, wort, flour or wholemeal, and/or
(ii) mixing barley grain, or processed barley grain, malt, wort, flour or wholemeal produced from the grain, with at least one other food or beverage ingredient, thereby producing the food or malt-based beverage ingredient, food or malt-based beverage,
wherein the barley grain has a reduced level of one or more alpha-amylase/trypsin inhibitors (ATIs) relative to grain from a corresponding wild-type barley plant.

2. The method of claim 1, wherein the one or more ATIs include at least one, at least two, at least three, at least four, at least five, at least six, or all of the following proteins:
(i) a protein comprising a sequence of amino acids provided as SEQ ID NO:86, or a sequence at least 95% identical thereto;
(ii) a protein comprising a sequence of amino acids provided as SEQ ID NO:87, or a sequence at least 95% identical thereto;
(iii) a protein comprising a sequence of amino acids provided as SEQ ID NO:88, or a sequence at least 95% identical thereto;
(iv) a protein comprising a sequence of amino acids provided as SEQ ID NO:89, or a sequence at least 95% identical thereto;
(v) a protein comprising a sequence of amino acids provided as SEQ ID NO:90, or a sequence at least 95% identical thereto;
(vi) a protein comprising a sequence of amino acids provided as SEQ ID NO:91, or a sequence at least 95% identical thereto; and
(vii) a protein comprising a sequence of amino acids provided as SEQ ID NO:92, or a sequence at least 95% identical thereto.

3. The method of claim 2, wherein the barley grain has one or more of the following properties:
(i) the level of the protein comprising a sequence of amino acids provided as SEQ ID NO:86, or a sequence at least 95% identical thereto, in the barley grain is about 20% or less, about 10% or less, about 5% or less, or about 2% or less of the level in the grain from the corresponding wild-type plant;
(ii) the level of the protein comprising a sequence of amino acids provided as SEQ ID NO:87, or a sequence at least 95% identical thereto, in the barley grain is about 25% or less, about 15% or less, about 10% or less, or about 5% or less of the level in the grain from the corresponding wild-type plant;
(iii) the level of the protein comprising a sequence of amino acids provided as SEQ ID NO:88, or a sequence at least 95% identical thereto, in the barley grain is about 50% or less, about 40% or less, about 30% or less, or about 20% or less of the level in the grain from the corresponding wild-type plant;
(iv) the level of the protein comprising a sequence of amino acids provided as SEQ ID NO:89, or a sequence at least 95% identical thereto, in the barley grain is about 50% or less, about 40% or less, about 30% or less, or about 20% or less of the level in the grain from the corresponding wild-type plant;
(v) the level of the protein comprising a sequence of amino acids provided as SEQ ID NO:90, or a sequence at least 95% identical thereto, in the barley grain is about 60% or less, about 50% or less, about 40% or less, or about 35% or less of the level in the grain from the corresponding wild-type plant;
(vi) the level of the protein comprising a sequence of amino acids provided as SEQ ID NO:91, or a sequence at least 95% identical thereto, in the barley grain is about 90% or less, about 80% or less, about 75% or less, or about 70% or less of the level in the grain from the corresponding wild-type plant; and
(vii) the level of the protein comprising a sequence of amino acids provided as SEQ ID NO:92, or a sequence at least 95% identical thereto, in the barley grain is about 90% or less, about 85% or less, about 80% or less, or about 75% or less of the level in the grain from the corresponding wild-type plant.

4. The method of any one of claims 1 to 3, wherein the barley grain comprises the following proteins in a summed level which is about 95% or less, about 90% or less, or about 85% or less of the level amount in grain from a corresponding wild-type plant:
(i) a protein comprising a sequence of amino acids provided as SEQ ID NO:92, or a sequence at least 95% identical thereto;
(ii) a protein comprising a sequence of amino acids provided as SEQ ID NO:93, or a sequence at least 95% identical thereto;
(iii) a protein comprising a sequence of amino acids provided as SEQ ID NO:94, or a sequence at least 95% identical thereto;
(iv) a protein comprising a sequence of amino acids provided as SEQ ID NO:91, or a sequence at least 95% identical thereto; and
(v) a protein comprising a sequence of amino acids provided as SEQ ID NO:86, or a sequence at least 95% identical thereto.

5. The method of any one of claims 1 to 4, wherein the barley grain comprises a summed level of the following proteins
(i) a protein comprising a sequence of amino acids provided as SEQ ID NO:92, or a sequence at least 95% identical thereto;
(ii) a protein comprising a sequence of amino acids provided as SEQ ID NO:93, or a sequence at least 95% identical thereto;
(iii) a protein comprising a sequence of amino acids provided as SEQ ID NO:94, or a sequence at least 95% identical thereto;
(iv) a protein comprising a sequence of amino acids provided as SEQ ID NO:91, or a sequence at least 95% identical thereto; and
(v) a protein comprising a sequence of amino acids provided as SEQ ID NO:86, or a sequence at least 95% identical thereto;
wherein the summed level is about 90% or less, about 80% or less, about 75% or less, or about 70% or less of the summed level in barley grain lacking C-hordeins.

6. The method according to any one of claims 1 to 5, wherein the barley grain has a reduced level of one or more or all of B-hordeins, C-hordeins, and D-hordeins, or any combinations thereof, relative to grain of the corresponding wild-type barley plant.

7. The method according to any one of claims 1 to 6, wherein the barley grain has a level of less than 10%, less than 5% or less than 2% of a wild-type level, or is lacking, one or more than one or all of:
i) B-hordeins comprising a sequence of amino acids provided as SEQ ID NO:53, ii) B-hordeins comprising a sequence of amino acids provided as SEQ ID NO:54,
iii) C-hordeins comprising a sequence of amino acids provided as SEQ ID NO:55, and
iv) D-hordeins comprising a sequence of amino acids provided as SEQ ID NO:56,
wherein each of the levels of less than 10%, less than 5% or less than 2% is relative to grain from a corresponding wild-type barley plant.

8. The method of claim 7, wherein the B-hordeins are at least B1-hordein and B3-hordein.

9. The method of claim 7 or claim 8, wherein the barley grain further has a level of less than 10%, less than 5% or less than 2% of a wild-type level, or is further lacking;
i) γ-hordeins comprising a sequence of amino acids provided as SEQ ID NO:57, and/or
ii) avenin-like A proteins comprising a sequence of amino acids provided as SEQ ID NO:52,
wherein each of the levels of less than 10%, less than 5% or less than 2% is relative to grain from a corresponding wild-type barley plant.

10. The method of any one of claims 1 to 9, wherein the barley grain has a reduced level of one or more of the following ATIs relative to grain from a corresponding wild-type barley plant:
(i) a protein comprising a sequence of amino acids provided as SEQ ID NO:93, or a sequence at least 95% identical thereto;
(ii) a protein comprising a sequence of amino acids provided as SEQ ID NO:94, or a sequence at least 95% identical thereto;
(iii) a protein comprising a sequence of amino acids provided as SEQ ID NO:96, or a sequence at least 95% identical thereto; and
(iv) a protein comprising a sequence of amino acids provided as SEQ ID NO:97, or a sequence at least 95% identical thereto.

11. The method of any one of claims 1 to 10, wherein the average weight of the barley grain is at least about 35 mg, at least about 39 mg, at least about 41 mg, at least about 47 mg, about 35 mg to about 60 mg, about 40 mg to about 60 mg, about 45 mg to about 60 mg, about 39.1 mg, about 41.8 mg or about 47.2 mg.

12. The method according to any one of claims 1 to 11, wherein at least about 80%, at least about 90%, at least about 95%, about 80% to about 98%, or about 80% to about 93%, of the barley grain do not pass through a 2.8 mm sieve.

13. The method according to any one of claims 1 to 12, wherein the barley grain is from a plant which has a harvest index of at least 40%, about 40% to about 60%, about 40% to about 55%, or about 40% to about 50%.

14. The method according to any one of claims 1 to 13, wherein the barley grain has a length to thickness ratio of less than about 5, less than about 4, less than about 3.8, about 2 to about 5, or about 2.5 to about 3.8.

15. The method according to any one of claims 1 to 14, wherein the barley grain is homozygous for an allele of the Hor2 locus where most or all of the B-hordein encoding genes have been deleted, or wherein the processed barley grain, malt, wort, flour or wholemeal produced from said grain comprises DNA which comprises the allele of the Hor2 locus where most or all of the B-hordein encoding genes have been deleted.

16. The method according to any one of claims 1 to 15, wherein the barley grain is homozygous for a null allele of the gene encoding D-hordein at the Hor3 locus, or wherein the processed barley grain, malt, wort, flour or wholemeal produced from said grain comprises DNA which comprises the null allele of the gene encoding D-hordein, the null allele preferably comprising a stop codon, splice site mutation, frame-shift mutation, insertion, deletion or encoding a truncated D-hordein, or where most or all of the D-hordein encoding gene has been deleted.

17. The method of claim 16, wherein the truncated D-hordein has a stop codon at the triplet encoding amino acid number 150.

18. The method according to any one of claims 1 to 17, wherein the barley grain is homozygous for an allele at the Lys3 locus of barley which results in the barley grain lacking C-hordeins, or wherein the processed barley grain, malt, wort, flour or wholemeal produced from said grain comprises DNA which comprises the allele at the Lys3 locus.

19. The method according to any one of claims 1 to 18, wherein the average grain weight is at least 1.05 fold, at least 1.1 fold, or 1.05 to 1.3 fold, higher than a grain which is
i) homozygous for an allele of the Hor2 locus where most or all of the B-hordein encoding genes have been deleted,
ii) homozygous for an allele at the Lys3 locus of barley which results in the barley grain lacking C hordeins, and
iii) homozygous for a wild-type allele of D hordein encoding a full-length protein.

20. The method according to any one of claims 1 to 19, wherein the barley grain is from a plant which has a grain yield which is least 1.20 fold, or at least 1.35 fold, or 1.2 to 1.5 fold, or 1.2 to 2.0 fold higher than the grain yield from a plant which is
i) homozygous for an allele of the Hor2 locus where most or all of the B-hordein encoding genes have been deleted,
ii) homozygous for an allele at the Lys3 locus of barley which results in the barley grain lacking C hordeins, and
iii) homozygous for a wild-type allele of D hordein encoding a full-length protein.

21. The method according to any one of claims 1 to 20, wherein at least about 50% of the genome of the barley grain is identical to the genome of a barley cultivar Sloop, Hindmarsh, Oxford or Maratime.

22. The method according to any one of claims 1 to 21, wherein the barley grain is from a plant comprising one or more genetic variations which reduce the level of the one or more ATIs in the barley grain relative to grain from the corresponding wild-type barley plant.

23. The method according to claim 22, wherein the barley grain is from a non-transgenic plant.

24. The method according to claim 22, wherein the barley grain is from a transgenic plant.

25. The method of claim 24, wherein the plant comprises a transgene which encodes a polynucleotide which down-regulates the production of at least one hordein and/or at least one ATI in the barley grain.

26. The method according to any one of claims 1 to 25 which comprises producing flour or wholemeal from the barley grain.

27. The method according to any one of claims 1 to 25 which comprises producing malt from the barley grain.

28. The method according to any one of claims 1 to 25, which comprises producing processed barley grain from the barley grain.

29. The method according to any one of claims 1 to 28, wherein the processed barley grain is dehulled barley or pearl barley.

30. The method according to any one of claims 1 to 29, wherein the malt-based beverage is beer and the method comprises germinating the barley grain or cracked grain derived therefrom.

31. The method of claim 30 which further comprises fractionating dried germinated grain into two or more of an endosperm fraction, an endothelial layer fraction, a husk fraction, an acrospire fraction, and a malt rootlets fraction; and combining and blending predetermined amounts of two or more of the fractions.

32. The method according to any one of claims 1 to 31, wherein at least about 50% of the barley grain germinates within 3 days following imbibition.

33. The method according to any one of claims 1 to 32, wherein the food ingredient or malt-based beverage ingredient is processed barley grain, flour, starch, malt, or wort, or wherein the food is processed barley grain, soup, stew, gruel, leavened or unleavened breads, pasta, noodles, breakfast cereals, snack foods, cakes, pastries or foods containing flour-based sauces.

34. The method according to any one of claims 1 to 33, wherein the malt-based beverage is beer or whiskey.

35. The method according to any one of claims 1 to 34, wherein the subject has non-coeliac gluten sensitivity (NCGS).

36. The method according to any one of claims 1 to 35, wherein the subject is a human.

37. The method according to any one of claims 1 to 36, wherein the subject does not have coeliac disease.

38. The method according to any one of claims 1 to 37, wherein the wild-type barley plant is Sloop, Hindmarsh, or Commander.

39. A product produced according to the method of any one of claims 1 to 38.

40. The product of claim 39, wherein the product is a food ingredient, malt-based beverage ingredient, food product or malt-based beverage product.

41. The product of claim 40, wherein the malt-based beverage product is beer or whiskey.

42. The product of claim 40, wherein the malt-based beverage ingredient is malt or wort.

43. The product of claim 40, wherein the food ingredient is processed barley grain, flour or wholemeal.

44. The product of claim 40, wherein the food is processed barley grain, soup, stew, gruel, leavened or unleavened bread, pasta, noodles, breakfast cereal, snack food, cake, pastry or a food containing a flour-based sauce.

45. A packaged product comprising
(i) the product of any one of claims 40 to 44, and
(ii) packaging which indicates that the product is suitable for consumption by a subject with a non-coeliac gastrointestinal sensitivity.

46. A method of feeding a subject with a non-coeliac gastrointestinal sensitivity, the method comprising providing the subject with a food or malt-based beverage produced from barley grain which has a reduced level of one or more alpha-amylase/trypsin inhibitors (ATIs) relative to grain from a corresponding wild-type barley plant.

47. A method of reducing the incidence or severity of a non-coeliac gastrointestinal sensitivity in a subject, the method comprising feeding the subject a food or malt-based beverage produced from barley grain which has a reduced level of one or more alpha-amylase/trypsin inhibitors (ATIs) relative to grain from a corresponding wild-type barley plant, wherein the reduction of the incidence or severity of the non-coeliac gastrointestinal sensitivity is relative to when the subject is fed the same amount of a corresponding food or malt-based beverage produced from grain from a corresponding wild-type barley plant.

48. Use of barley grain which has a reduced level of one or more alpha-amylase/trypsin inhibitors (ATIs) relative to grain from a corresponding wild-type barley plant in the manufacture of a food or malt-based beverage for reducing the incidence or severity of a non-coeliac gastrointestinal sensitivity in a subject, wherein the reduction of the incidence or severity of the non-coeliac gastrointestinal sensitivity is relative to when the subject is fed the same amount of a corresponding food or malt-based beverage produced from grain from a corresponding wild-type barley plant.

49. Use of barley grain which has a reduced level of one or more alpha-amylase/trypsin inhibitors (ATIs) relative to grain from a corresponding wild-type barley plant in the manufacture of a food or malt-based beverage for consumption by a subject with a non-coeliac gastrointestinal sensitivity.

50. A food or malt-based beverage produced from barley grain which has a reduced level of one or more alpha-amylase/trypsin inhibitors (ATIs) relative to grain from a corresponding wild-type barley plant, for use in reducing the incidence or severity of a non-coeliac gastrointestinal sensitivity in a subject, wherein the reduction of the incidence or severity of the non-coeliac gastrointestinal sensitivity is relative to when the subject is fed the same amount of a corresponding food or malt-based beverage produced from grain from a corresponding wild-type barley plant.

51. Barley grain which is homozygous for an allele at the Lys3 locus of barley which results in the barley grain lacking C-hordeins, wherein the barley grain has a reduced level of one or more of the following alpha-amylase/trypsin inhibitors (ATIs) relative to grain from a corresponding wild-type barley plant:
(i) a protein comprising a sequence of amino acids provided as SEQ ID NO:93;
(ii) a protein comprising a sequence of amino acids provided as SEQ ID NO:94;
(iii) a protein comprising a sequence of amino acids provided as SEQ ID NO:96; and
(iv) a protein comprising a sequence of amino acids provided as SEQ ID NO:97.

52. The barley grain of claim 51, wherein
(i) the barley grain is homozygous for an allele of the Hor2 locus where most or all of the B-hordein encoding genes have been deleted, and/or
(ii) the barley grain is homozygous for a null allele of the gene encoding D-hordein at the Hor3 locus.
